(19) United States
(12) Patent Application Publication
(10) Pub. No.: US 2002/0049155 A1
(43) Pub. Date: Apr. 25, 2002

(54) Cobalamin compounds useful as cardiovascular agents and as imaging agents

(76) Inventor: Henricus P.C. Hogenkamp, Roseville, MN (US)

Correspondence Address: KING & SPALDING, 191 PEACHTREE STREET, N.E., ATLANTA, GA, 30303-1763, US

(21) Appl. No.: 09/873,142

(22) Filed: May 31, 2001

Related U.S. Application Data
(60) Provisional application No. 60/208,140, filed on May 31, 2000.
(60) Provisional application No. 60/267,782, filed on Feb. 9, 2001.

(51) Int. Cl. A61K 38/16

(52) U.S. Cl. 514/7

(57) ABSTRACT

The invention provides cobalamin derivatives linked to a cardiovascular agent, as well as pharmaceutical compositions comprising the compounds and methods for using the compounds in treatment or diagnosis of a cardiovascular disease.

This application claims priority to U.S. provisional application no. 60/208,140, filed on May 31, 2000 and U.S. provisional application no. 60/267,782, filed on Feb. 9, 2001.

### FIELD OF THE INVENTION

This invention provides compounds, compositions and methods for treating microbial infection.

### BACKGROUND OF THE INVENTION

According to statistics compiled by the Centers for Disease Control, almost one in two Americans dies of cardiovascular disease. The annual toll is more than 975,000; of these, about 500,000 die of heart attacks. The large majority of heart attacks results from coronary artery disease, a condition that afflicts about 5 million Americans. Of course, mortality statistics are only part of the story, coronary artery disease also affects lifestyle, productivity and the economy. According to 1991 figures compiled by the American Heart Association and the National Center for Health Statistics, about 6 million Americans have a history of a heart attack, angina or both. Though the likelihood of a heart attack increases with age, a large number of Americans, mostly men, are struck down during their most productive years. About 45 percent of heart attacks occur before the age of 65, with 5 percent before age 40. The American Heart Association puts the total annual cost of cardiovascular disease at $94.5 billion, a figure that includes both direct medical costs and estimated lost productivity resulting from disability.

Although the body's entire volume of blood passes through the heart's chambers approximately every 60 seconds, only about 5 percent of the total amount of oxygenated blood is available for the heart's own energy needs. The coronary arteries (which are 3 to 5 millimeters or ⅛ to ⅕ of an inch in diameter) are the sole conduits for this supply. Because heart muscle (myocardium) extracts oxygen from arterial blood with maximum efficiency, any increase in the heart's workload requires an increase in the blood supply. When there is an imbalance between the available supply of blood (oxygen) and demand for blood (oxygen), the 30 heart muscle becomes deprived of oxygen, a condition known as myocardial ischemia. Without adequate blood flow to the heart muscle, the heart itself is unable to function properly.

For the majority of people suffering from coronary artery disease, blood oxygen supply is reduced by a progressive narrowing of the open channels (the interior lumens) of the coronary arteries. This narrowing of the arteries is due to atherosclerosis, a disease in which scattered lesions, known as atherosclerotic plaques or atheromas, appear on the inner wall of the coronary artery.

The first signs of atherosclerosis can appear at an early age. A significant proportion of males in their teens and early twenty's already may have fatty streaks and other evidence of the disease on the walls of their coronary arteries. However, the gradual buildup of atherosclerotic plaque, from the first appearance of fatty streaks to coronary arteries blocked enough to produce symptoms such as angina or shortness of breath and, may take upward of 20 years or more. Symptoms usually do not occur until the coronary artery is narrowed by about 50 to 70 percent. Insufficiency of blood supply may result from a reduction of blood flow through one or more of these arteries. Heart cells are dependent on blood flow through these arteries to provide oxygen and to carry away metabolic products. Without an adequate flow of blood, these cells can become injured or die. When this occurs, immediate emergency treatment is necessary to stop the injury from widening, killing additional heart cells and increasing the risk of complications or death.

Even with significantly clogged coronary arteries causing ischemia, however, many people do not experience symptoms. The exact causes of buildup of atherosclerotic plaque is not understood, nor is it possible to pinpoint how they begin or what course they will be. In addition to atherosclerotic plaque buildup, spasms of the muscles that encircle the coronary arteries can also interrupt the coronary blood supply. Additionally, in 85 percent of people who have coronary artery spasms, atherosclerosis is also present.

Cardiovascular Agents

A “cardiovascular agent” is any compound useful to treat one or more abnormal conditions associated with the cardiovascular system. Suitable cardiovascular agents are disclosed, e.g. in Physician's Desk Reference (PDR), Medical Economics Company (Montvale, N.J.), (53rd Ed.), 1999; Maya Medical Center Formulary., Unabridged Version, Mayo Clinic (Rochester, Minn.), January 1998; Yale University School of Medicine Heart Book: Chapter 23, Cardiovascular Drugs, http://www.info.med.yale.edu/library/heartbk, Apr. 16, 1999; Merck Index, An Encyclopedia of Chemicals, Drugs and Biologicals, (11th Ed.), Merck & Co., Inc. (Rahway, N.J.), 1989; and references cited therein.

Suitable cardiovascular agents include blood modifiers, adrenergic blockers (peripheral), adrenergic stimulants (central), alphalbeta adrenergic blockers, angiotensin converting enzyme (ACE) inhibitors, angiotensin II receptor antagonists, anti-arrhythmics (groups I, II, III and IV), miscellaneous anti-arrhythmics, 30 anti-lipemic agents, beta adrenergic blocking agents, calcium channel blockers, diuretics, hypertensive emergency agents, inotropic agents, miscellaneous cardiovascular agents, rauwolfia derivatives, vasodilators and vasopressors.

Suitable blood modifiers include anticoagulants (e.g. Coumadin (crystalline warfarin sodium); Fragmin (dalteparin sodium injection); Heparin Lock (heparin lock flush solution); Heparin sodium (heparin sodium); Lovenox (enoxaparin sodium); Normiflo (ardeparin sodium); Orgaran (danaparoid sodium)); antiplatelet agents (e.g. Aggrastat (tirofiban hydrochloride monohydrate); Agrylin (anagrelide hydrochloride); Ecotrin (enteric-coated aspirin); Flolan (epoprostenol sodium); Halfprin (enteric-coated aspirin); Integrilin (eptifibatide); Persantine (dipyridamole); Plavix (clopidogrel bisulfate); ReoPro (abciximab); and Ticlild (ticlopidine hydrochloride)); colony stimulating factors (e.g. Granulocyte colony-stimulating factor (G-CSF) such as Neupogen (filgrastim); Granulocyte-Macrophage colony-stimulating factor (GM-CSF), such as Leukine (sagramostim)); and hematinics (e.g. Anabolic steroids, such as Anadrol-50 (oxymetholone); and Nascobal (cyanocobalamin); and Trinsicon (hematinic concentrate with intrinsic factor); and Erythropoietin, such as Epogen (epoetin alfa); and Procrit (epoetin alfa).

Suitable adrenergic blockers (peripheral) include Cardura (doxazosin mesylate); Dibenzyline (phenoxybenzamine); Hylorel (guanadrel sulfate); Hytrin (terazosin hydrochloride); Minipress (prazosin hydrochloride); and Minizide (prazosin hydrochloride/polythiazide).

Suitable adrenergic stimulants (central) include Aldoclor (methyldopa and chlorothiazide sodium); Aldomet (methyldopa); Aldomet ester HCL (methyldopate HC1); Aldoril (methyldopa and hydrochlorothiazide); Catapres (clonidine HC1); Catapres-TTS (clonidine); Clorpres (clonidine hydrochloride and 25 chlorthalidone); Combipres (clonidinehydrochloride and chlorthalidone); and Tenex (guanfacine).

Suitable alpha/beta adrenergic blockers include Coreg (carvedilol); Normodyne (Labetalol); and Trandate (Labetalol).

Suitable angiotensin converting enzyme (ACE) inhibitors include 30 Accupril (quinapril hydrochloride); Altace (ramipril); Captopril; Lotensin (benazepril hydrochloride); Mavik (trandolapril); Monopril (fosinopril sodium tablets); Prinivil (Lisinopril); Univasc (moexipril hydrochloride); Vasotec (enalapril maleate); and Zestril (lisinopril).

Suitable angiotensin II receptor antagonists include Atacand (candesartan cilexetil); Avapro (irbesartan); Cozaar (losartan potassium); and Diovan (Valsartan) HCT™ (Hydrochlorothiazide).

Suitable anti-arrhythmics, group I include Cardioquin (quinidine polygalacturonate); Ethmozine (moricizine hydrochloride); Mexitil (mexiletine hydrochloride); Norpace (disopyramide phosphate); Norpace CR (controlled release disopyrarnide phosphate); Procanbid (procainamide hydrochloride extended-release tablets); Quinaglute (Quinidine); Quinidex (quinidine sulfate); Rythmol (propafenone hydrochloride); Tambocor (flecainide acetate); and Tonocard (tocainide HCL). Suitable anti-arrhythmics, group II include Betapace (sotalol HCL); Brevibloc (esmolol hydrochloride); Inderal (Popranolol); and Sectral (acebutolol).

Suitable anti-arrhythmics, group III include Betapace (sotalol HCL); Cordarone (amiodarone); Corvert (ibutilide fumarate injection); and Pacerone (Amiodarone hydrochloride).

Suitable anti-arrhythmics, group IV include Calan (verapamil); and Cardizem (diltiazem HCL).

Suitable miscellaneous anti-arrhythmics include Adenocard (adenosine); Lanoxicaps (digoxin); and Lanoxin (digoxin).

Suitable anti-lipemic agents include bile acid sequestrants (e.g. Colestid (microionized colestipol hydrochloride); LoCholest (cholestyramine); and Questran (cholestyramine)); fibric acid derivatives (e.g Atromid-S (clofibrate); Lopid (gemfibrozil); and TriCor (fenofibrate capsules)); HMG-CoA reductase inhibitors (e.g. Baycol (cerivastatin sodium tablets); Lescol (fluvastatin sodium); Lipitor (atorvastatin calcium); Mevacor (lovastatin); Pravachol (pravastatin sodium); and Zocor (simvastatin)); and Nicotinic Acid (e.g. Niaspan).

Suitable beta adrenergic blocking agents include Betapace (sotalol HC1); Blocadren (Timolol Maleate); Brevibloc (esmolol hydrochloride); Cartrol (carteolol hydrochloride); Inderal (propranolol hydrochloride); Kerlone (betaxolol hydrochloride); Levatol (Penbutolol sulfate); Lopressor (metropolol tartrate); Sectral (acebutolol hydrochloride); Tenormin (atenolol); Toprol-XL (metoprolol succinate, extended release); and Zebeta (bisoprolol fumurate).

Suitable calcium channel blockers include Adalat (nifedipine); Adalat CC (nifedipine); Calan (verapamil hydrochloride); Calan SR (verapamil hydrochloride); Cardene (nicardipine hydrochloride); Cardizem CD (diltiazem hydrochloride); Cardizem (diltiazem hydrochloride); Cardizem SR (diltiazem hydrochloride); Covera-HS (verapamil hydrochloride); Dilator XR (dilitiazem); DynaCirc (isradipine); DynaCirc CR (isradipine); Isoptin SR (verapamil hydrochloride); Nimotop (nimodipine); Norvasc (amlodipine besylate); Plendil (felodipine); Procardia (nifedipine); Procardia XL (nifedipine, extended release); Sular (nisoldipine); Tiazac (diltiazem hydrochloride); Vascor (bepridil hydrochloride); and Verelan (Vempamil Hydrochloride).

Suitable diuretics include carbonic anhydrase inhibitors (e.g. Daranide (dichlorphenamide)); loop diuretics (e.g. Demadex (torsemide); Edecrin (ethacrynic acid); Edecrin sodium (ethacrynic acid); and Lasix (furosemide)); 20 potassium-sparing diuretics (e.g. Aldactone (Spironolactone); Dyrenium (triamterene); and Midamor (amiloride)); thiazides and related diuretics (e.g. Diucardin (hydroflumethazide); Diuril (chlorothiazide); Diuril sodium (chlorothiazide); Enduron (methyclothiazide); HydroDIURIL (hydrochlorothiazide (HCTZ)); Microzide (hydrochlorothiazide); Mykrox (metolazone); Renese (polythiazide); Thalitone (chlorthalidone USP); and Zaroxolyn (metolazone)).

Suitable hypertensive emergency agents include Hyperstat (diazoxide).

Suitable inotropic agents include Dobutrex (dobutamine hydrochloride); Lanoxicaps (digoxin); and Lanoxin (digoxin); and Primacor (milrinone lactate injection).

Suitable miscellaneous cardiovascular agents include Demser (metyrosine); Inversine (Mecamylamine HCL); Regitine (phentolamine mesylate); and ReoPro (abciximab).

Suitable rauwolfia derivatives include Diupres (reserpine and chlorothiazide); and Hydropres (reserpine and hydrochlorothiazide).

Suitable vasodilators include coronary vasodilators (e.g. Deponit (Transdermal Nitroglycerin); Dilatrate-SR (isosorbide dinitrate sustained release); Imdur (isosorbide mononitrate); Ismo (isosorbide mononitrate); Isordil (isosorbide dinitrate); Monoket (isosorbide mononitrate); Nitro-Bid (nitroglycerin); Nitro-Dur (nitroglycerin); Nitrolingual (Nitroglycerin in propellants, Dichlorodifluoromethane and Dichlorotetrafluoromethane); Nitrostat (nitroglycerin); Sorbitrate (isosorbide dinitrate); and Transderm-Nitro (nitroglycerin)); peripheral vasodilators (e.g. Corlopam (fenoldopam mesylate); Flolan (epoprostenol sodium); and Primacor (milrinone lactate injection)).

Suitable vasopressors include Ana-Kit (epinephrine); Aramine (Metaraminol bitartrate); EpiPen (epinephrine); ProAmatine (midodrine hydrochloride); and Vasoxyl (methoxamine hydrochloride).

Coumadin (crystalline warfarin sodium) is commercially available from DuPont and is 3-(α-acetonylbenzyl)-4-hydroxycoumarin.

Fragmin (dalteparin sodium injection) is commercially available from Pharmacia & Upjohn and is a low molecular weight heparin.

Heparin Lock (heparin lock flush solution) is commercially available from Elkins-Sinn.

Heparin sodium (heparin sodium) is commercially available from Wyeth-Ayerst and is heparin, a heterogeneous group of straight-chain anionic mucopolysaccharides called glycoaminoglycans.

Lovenox (enoxaparin sodium) is commercially available from Rh6ne-Poulenc Rorer and is the sodium salt of enoxaparin, which is characterized by a 2-O-sulfo-4-enepyranosuronic acid group at the non-reducing end and a 2-N,6-30 O-disulfo-D-glucosamine at the reducing end of the chain.

Normiflo (ardeparin sodium) is commercially available from Wyeth-Ayerst and is smaller polymer chains consisting of derivatives of D-glucosamine (N-sulfated, N-acetylated, and/or O-sulfated) and hexuronic acid (L-iduronic acid or D-glucuronic acid, including D-sulfated derivatives.

Orgaran (danaparoid sodium) is commercially available from Organon.

Aggrastat (tirofiban hydrochloride monohydrate) is commercially available from Merck and Company, Inc. and is N-(butylsulfonyl)-O-[4-(4-piperidinyl)butyl]-L-tyrosine monohydro-chloride monohydrate.

Agrylin (anagrelide hydrochloride) is commercially available from Roberts Pharmaceutical Corp. and is 6,7-dichloro-1,5-dihydroimidazo [2,1-b] quinazolin-2 (3H)-one monohydrochloride monohydrate.

Ecotrin (enteric-coated aspirin) is commercially available from SmithKline Beecham and is acetylsalicylic acid.

Flolan (epoprostenol sodium) is commercially available from Glaxo Wellcome and is 5Z,9 α, 11α, 13E,15S)-6,9-epoxy-11,15-dihydroxy prosta-5,13-dien-1-oic acid.

Halfprin (enteric-coated aspirin) is commercially available from Kramer and is acetylsalicylic acid.

Integrilin (eptifibatide) is commercially available from COR Therapeutics and is N⁶-(aminoiminomethyl)-N²-(3-mercapto-1-oxopropyl-L-lysylglycyl-L-tryptophyl-L-prolyl-L-cysteinamaide, cyclic (1→6)-disulfide.

Persantine (dipyridamole) is commercially available from Boerhinger Ingelheim and is 2,6-bis-(diethanolamino)-4,8-dipiperidino-pyrimido-(5,4-d) pyrimidine.

Plavix (clopidogrel bisulfate) is commercially available from Bristol-Myers Squibb and is methyl (+)-(S)-α-(2-chlorophenyl)-6,7-dihydrothieno[3,2-c]pyridine-5(4H)-acetate sulfate (1:1).

ReoPro (abciximab) is commercially available from Lilly and is the FAB fragment of the chimeric human-murine monoclonal antibody 7E3.

Ticlild (ticlopidine hydrochloride) is commercially available from Roche Pharmaceuticals and is 5-[(2-chlorophenyl)methyl]-4,5,6,7-tetrahydrothieno[3,2-c] pyridine hydrochloride).

Neupogen (filgrastim) is commercially available from Amgen and is a recombinant human G-CSF.

Leukine (sagramostim) is commercially available from Immunex and is a recombinant human GM-CSF.

Anadrol-50 (oxymetholone) is commercially available from Unimed and is 17β-hydroxy-2-(hydroxymethylene)-17-methyl-5α-androstan-3-one.

Nascobal (cyanocobalamin) is commercially available from Schwarz Pharma and is 5,6-dimethyl-benzimidazolyl cyanocobamide.

Trinsicon (hematinic concentrate with intrinsic factor) is commercially available from UCB Pharma.

Erythropoietin, such as Epogen (epoetin alfa) is commercially available from Amgen and is a recombinant human erythropoeitin.

Procrit (epoetin alfa) is commercially available from Ortho Biotech and is a recombinant human erythropoietin.

Cardura (doxazosin mesylate) is commercially available from Pfizer and is 1-(4-amino-6,7-dimethoxy-2-quinazolinyl)-4-(1,4-benzodioxan-2-ylcarbonyl) piperazine methanesulfonate.

Dibenzyline (phenoxybenzamine) is commercially available from SmithKline Beecham and is N-(2-Chloroethyl)-N-(1-methyl-2-phenoxyethyl) benzylamine hydrochloride.

Hylorel (guanadrel sulfate) is commercially available from Medeva and is (1,4-Dioxaspiro[4,5] dec-2-ylmethyl) guanidine sulfate.

Hytrin (terazosin hydrochloride) is commercially available from Abbott and is (RS)-Piperazine, 1-(4-amino-6,7-dimethoxy-2-quinazolinyl)-4-[(tetra-hydro-2-furanyl)carbonyl]-monohydrochloride, dihydrate.

Minipress (prazosin hydrochloride) is commercially available from Pfizer and is 1-(4-amino-6,7-dimethoxy-2-quinazolinyl-4-(2-furoyl) piperazine.

Minizide (prazosin hydrochloride/polythiazide) is commercially available from Pfizer and is 1-(4-amino-6,7-dimethoxy-2-quinazolinyl)-4-(2-furoyl) piperazine/2H-1,2,4-Benzothiadiazine-7-sulfonamide, 6-Chloro-3,4-dihydro-2-methyl-3-[[2,2,2-trifluoroethyl)-thio]methyl]-1,1-dioxide.

Aldoclor (methyldopa and chlorothiazide sodium) is commercially available from Merck and is levo-3-(3,4-dihydroxyphenyl)-2-methylalanine (methyldopa) and 6-chloro-2H-1,2,4-benzothiadiazine-7-sulfonamide 1,1-dioxide (chlorothiazide).

Aldomet (methyldopa) is commercially available from Merck and is levo-3-(3,4-dihydroxyphenyl)-2-methylalanine.

Aldomet ester HCL (methyldopate HC 1) is commercially available from Merck and is levo-3-(3,4-dihydroxyphenyl)-2-methylalanine, ethyl ester hydrochloride.

Aldoril (methyldopa and hydrochlorothiazide) is commercially available from Merck and is levo-3-(3,4-dihydroxyphenyl)-2-methylalanine (methyldopa) and 6-chloro-3,4-dihydro-2H-1,2,4-benzothiadiazine-7-sulfonamide 1,1-dioxide (hydrochlorothiazide).

Catapres (clonidine HCI) is commercially available from Boerhinger Ingelheim and is 2-[(2,6-dichlorophenyl)imino]imidazoline monohydrochloride.

Catapres-TTS (clonidine) is commercially available from Boerhinger Ingelheim and is 2,6-dichloro-N-2-imidazolidinylidenebenzenamine.

Clorpres (clonidine hydrochloride and chlorthalidone) is commercially available from Bertek and is 2-[(2,6-dichlorophenyl)imino]-imidazoline monohydrochloride and 2-chloro-5-(1-hydroxy-3-oxo-1-isoindolinyl) benzenesulfonamide.

Combipres (clonidinehydrochloride and chlorthalidone) is commercially available from Boerhinger Ingelheim and is 2-(2,6-dichlorophenylamino)-2-imidazoline hydrochloride and 2-chloro-5-(1-hydroxy-3-oxo-1-isoindolinyl) benzenesulfonamide.

Tenex (guanfacine) is commercially available from A.H. Robins Co. and is N-amidino-2-(2,6-dichlorophenyl) acetamide hydrochloride.

Coreg (carvedilol) is commercially available from SmithKline Beecham and is (±)-1-(Carbazol-4-yloxy)-3-[[2-(o-methoxyphenoxy)ethyl]amino]-2-propanol.

Normodyne (Labetalol) is commercially available from Schering and is 2-hydroxy-5-[1-hydroxy-2-[(1-methyl-3-phenyl-propyl)amino]ethyl]benzamide monohydrochloride.

Trandate (Labetalol) is commercially available from Glaxo Wellcome and is 2-hydroxy-5-[1-hydroxy-2-[(1-methyl-3-phenyl-propyl)amino]ethyl]benzamide monohydro-chloride.

Accupril (quinapril hydrochloride) is commercially available from Parke-Davis and is [3S-2[R*(R*)],3R*]]-2-[2-[[1-(ethoxycarbonyl)-3-phenylpropyl]amino]-1-oxopropyl]-1,2,3,4-tetrahydro-3-isoquinolinecarboxylic acid, monohydrochloride.

Altace (ramipril) is commercially available from Hoechst Marion Roussel and is (2S,3aS,6aS)-1 [(S)-N-[(S)-1-carboxy-3-phenyl-propyl]alanyl]-octahydrocyclopenta[b]-pyrrole-2-carboxylic acid, 1-ethyl ester.

Captopril, which is commercially available from Mylan Pharmaceuticals and is 1-[(2S)-3-mercapto-2-methylpropionyl]-L-proline).

Lotensin Coenazepril hydrochloride) is commercially available from Novartis and is 3-[[1-(ethoxy-carbonyl)-3-phenyl-(1 S)-propyl]amino]-2,3,4,5-tetrahydro-2-oxo-1H-1-(3S)-benzazepine-1-acetic acid monohydrochloride.

Mavik (trandolapril) is commercially available from Knoll Pharmaceuticals and is (2S,3aR,7aS)-1-[(S)-N-[(S)-1-carboxy-3-phenylpropyl] -alanyl]hexahydro-2-indolinecarboxylic acid, 1-ethyl ester.

Monopril (fosinopril sodium tablets) is commercially available from Bristol-Myers Squibb and is trans-4-cyclohexyl-1-[[[2-methyl-1-(1-oxopropoxy)propoxy](4-phenylbutyl)-phosphinyl]acetyl]-L-proline, sodium salt.

Prinivil (Lisinopril) is commercially available from Merck and is (S)-1-[N²-(1-carboxy-3-phenylpropyl-L-lysyl]-L-proline dehydrate.

Univasc (moexipril hydrochloride) is commercially available from Schwarz Pharma and is [3S-[2[R*(R*)],3R*]]-2-[2-[[1-(ethoxycarbonyl)-3-phenylpropyl]amino]-1-oxo-propyl]-1,2,3,4-tetrahydro-6,7-dimethoxy-3-isoquinolinecarboxylic acid, monohydro-chloride.

Vasotec (enalapril maleate) is commercially available from Merck and is (S)-1-[N-[1-(ethoxycarbonyl)-3-phenylpropyl]-L-alanyl]-L-proline, (Z)-2-butenedioate salt (1:1).

Zestril (lisinopril) is commercially available from Zeneca and is (S)-1-[N²-(1-carboxy-3-phenylpropyl)-L-lysyl]-L-proline dihydrate.

Atacand (candesartan cilexetil) is commercially available from Astra Phannaceuticals and is (±)-1-[[(cyclohexyloxy)carbonyl]oxy]ethyl 2-ethoxy-1-[[2′-(1H-tetrazol-5-yl) [1,1 ′-biphenyl]-4-yl]methyl]1H-benzimidazole-7-carboxylate.

Avapro (irbesartan) is commercially available from Bristol-Myers Squibb and is 2-butyl-3-[[2′-(1H-tetrazol-5-yl) [1,1′-biphenyl]-4-yl]methyl]-1,3-diazaspiro [4,4] non-1-en-4-one.

Cozaar (losartan potassium) is commercially available from Merck and is 2-butyl-4-chloro-1-[p-(o-1H-tetrazol-5-ylphenyl)benzyl]imidazole-5-methanol monopotassium salt.

Diovan (Valsartan) HCT™ (Hydrochlorothiazide) is commercially available from Novartis and is N-(1-oxopentyl)-N-[[2′-(1H-tetrazol-5-yl) [1,1′-biphenyl]-4-yl]methyl]-L-valine.

Cardioquin (quinidine polygalacturonate) is commercially available from Purdue Frederick and is a polymer of quinidine and galacturonic acid.

Ethmozine (moricizine hydrochloride) is commercially available from A.H. Roberts and is 10-(3-morpholinopropionyl) phenothiazine-2-carbamic acid ethyl ester hydrochloride.

Mexitil (mexiletine hydrochloride) is commercially available from Boehringer Ingelheim and is 1-methyl-2-(2,6-xylyloxy)ethylamine hydrochloride.

Norpace (disopyramide phosphate) is commercially available from Searle and is α-1-[2-(diisopropylamino) ethyl]-α-phenyl-2-pyfidineacetamide phosphate.

Procanbid (procainamide hydrochloride extended-release tablets) is commercially available from Monarch and is p-amino-N-[2-(diethylamino) ethyl]benzamide monohydro-chloride.

Quinaglute (Quinidine) is commercially available from Bertek and is (9S)-cinchonan-9-ol, 6′-methoxy-mono-D-gluconate.

Quinidex (quinidine sulfate) is commercially available from A.H. Robins and is (9S)-cinchonan-9-ol, 6′-methoxy-sulfate (2:1) dehydrate.

Rythmol (propafenone hydrochloride) is commercially available from Knoll Labs and is 2′-[2-Hydroxy-3-(propylamino)-propoxy]-3-phenylpropiophenone hydrochloride.

Tambocor (flecainide acetate) is commercially available from 3M Pharmaceuticals and is benzamide, N-(2-piperidinylmethyl)-2,5-bis (2,2,2-trifluoroethoxy)-monoacetate.

Tonocard (tocainide HC1) is commercially available from Astra Pharmaceuticals and is 2-amino-N-(2,6-dimethylphenyl) propanamide hydrochloride.

Betapace (sotalol HCL) is commercially available from Berlex Laboratories and is α-1-N-[4-[1-hydroxy-2-[(-methylethyl)amino]ethyl]-phenyl]methane-sulfonamide monohydrochloride.

Brevibloc (esmolol hydrochloride) is commercially available from Baxter Pharmaceutical Products Inc. and is (+)-Methyl p-[2-hydroxy-3-(isopropylamino) propoxy]hydrocinnamate hydrochloride.

Inderal (Popranolol) is commercially available from Wyeth-Ayerst and is 1-(isopropylamino)-3-(1-naphthyloxy)-2-propanol hydrochloride.

Sectral (acebutolol) is commercially available from Wyeth-Ayerst and is the hydrochloride salt +N-[3-Acetyl-4-[2-hydroxy-3-[(1-methylethyl)amino]propoxy]phenyl] butanamide or (±)-3′-Acetyl-4′-[2-hydroxy-3-(isopropylamino) propoxy] butyranilide.

Betapace (sotalol HCL) is commercially available from Berlex and is 1,1-N-[4-[1-hydroxy-2-[(1-methylethyl)amino]ethyl]phenyl]methane-sulfonamide monohydro-chloride.

Cordarone (amiodarone) is commercially available from Wyeth-Ayerst and is 2-butyl-3-benzofuranyl 4-[2-(diethylamino)-ethoxy]-3,5-diiodophenyl ketone hydrochloride.

Corvert (ibutilide fumarate injection) is commercially available from Pharmacia & Upjohn and is Methane-sulfonamide, N-{4-{4-(ethyl-heptylamino)-1-hydroxy butyl}phenyl}, (+), (−), (E)-2-butenedioate (1:0.5) (hemifumarate salt).

Pacerone (Amiodarone hydrochloride) is commercially available from Upsher-Smith and is 2-butyl-3-benzofuranyl 4-[2-(diethylamino)-ethoxy]-3,5-diiodophenyl ketone hydrochloride.

Calan (verapamil) is commercially available from Searle and is Benzeneacetonitrile, α-[[3-[[2-(3,4-dimethoxyphenyl)ethyl]methylamino]propyl]-3,4-dimethoxy-α-(1-methyl-ethyl)hydrochloride.

Cardizem (diltiazem HCl) is commercially available from Hoechst Marion Roussel and is (+)-cis-1,5-benzothiazepin-4(5H)one,3-(acetyloxy)-5-[2-(dimethylamino)ethyl]-2,3-dihydro-2-(4-methoxyphenyl)-monohydrochloride.

Adenocard (adenosine) is commercially available from Fujisawa Pharmaceutical Co., Ltd. and is 6-amino-9-β-D-ribofuranosyl-9-H-purine.

Lanoxicaps (digoxin) is commercially available from Glaxo Wellcome and is (3β,5β, 12β)-3-[(O-2,6-dideoxy-β-D-ribo-hexopyranosyl-(1→4)-O-2,6-dideoxy-β-D-ribo-hexopyranosyl-(1→4)-2,6-dideoxy-β-D-ribo-hexopyranosyl)oxy]-12,14-dihydroxy-card-20(22)-enolide.

Lanoxin (digoxin) is commercially available from Glaxo Wellcome and is (3β,5β,12β)-3-[(O-2,6-dideoxy-β-D-ribo-hexopyranosyl-(1→4)-O-2,6-dideoxy-β-D-ribo-hexopyranosyl-(1→4)-2,6-dideoxy-β-D-ribo-hexopyranosyl)oxy]-12,14-dihydroxy-card-20(22)-enolide.

Colestid (microionized colestipol hydrochloride) is commercially available from Pharmacia & Upjohn.

LoCholest (cholestyramine) is commercially available from Warner Chilcott Professional Products.

Questran (cholestyramine) is commercially available from Bristol-Myers Squibb.

Atromido-S (clofibrate) is commercially available from Wyeth-Ayerst and is ethyl 2-(p-chlorophenoxy)-2-methyl-propionate.

Lopid (gemfibrozil) is commercially available from Parke-Davis and is 5-(2,5-dimethylphenoxy)-2,2-dimethyl pentanoic acid.

TriCor (fenofibrate capsules) is commercially available from Abbott and is 2-[4-(4-chlorobenzoyl)phenoxy]-2-methyl-propanoic acid, 1-methyl ethyl ester.

Baycol (cefivastatin sodium tablets) is commercially available from Bayer Pharma and is sodium [S-[R*,S*-(E)]]-7-[4-(4-fluorophenyl)-5-methoxymethyl)-2,6-bis(1-methyl-ethyl)-3-pyridinyl]-3,5-dihydroxy-6-heptenoate.

Lescol (fluvastatin sodium) is commercially available from Novartis and is [R*,S*-(E)]-(±)-7-[3-(4-fluorophenyl)-1-(1-methylethyl)-1H-indol-2-yl]-3,5-dihydroxy-6-heptenoic acid, monosodium salt.

Lipitor (atorvastatin calcium) is commercially available from Parke Davis and is [R-(R*,R*)]-2-(4-fluorophenyl)-β,δ-dihydroxy-5-(1-methylethyl)-3-phenyl-4-[(phenyl-amino)carbonyl]-1H-pyrrole-1-heptanoic acid, calcium salt (2:1) trihydrate.

Mevacor (lovastatin) is commercially available from Merck and is [1S-[1-α-(R*),3-α,7β,8β(2S ,4S),8aB]]-1,2,3,7,8,8a-hexahydro-3,7-dimethyl-8-[2-(tetrahydro-4-hydroxy-6-oxo-2H -pyran-2-yl)ethyl]-1-naphthalenyl-2-menthylbutanotate.

Pravachol (pravastatin sodium) is commercially available from Bristol-Myers Squibb and is [1S-[1α(βS*,εS*),2α, 6α,8α(R*),8aα]]-1,2,6,7,8,Sa-hexahydro-β,δ,6-tri-hydroxy-2-methyl-8-(2-methyl-1-oxobutoxy)-1-naphthalene-heptanoic acid monosodium salt.

Zocor (simvastatin) is commercially available from Merck and is butanoic acid, [1S-1α,3α,7β,8β(2S*,4S*),-8aβ]]-2,2-dimethyl-1,2,3,7,8,8a-hexahydro-3,7-dimethyl-8-[2-(tetrahydro-4-hydroxy-6-oxo-2H-pyran-2-yl)-ethyl]-1-naphthalenyl ester.

Nicotinic Acid (e.g. Niaspan) is commercially available from Kos.

Betapace (sotalol HCL) is commercially available from Berlex Laboratories and is α1-N-[4-[1-hydroxy-2-[(1-methylethyl)amino]ethyl]phenyl]-methane-sulfonamide mono-hydrochloride.

Blocadren (Timolol Maleate) is commercially available from Merck and is (S)-1-[(1,1-di-methylethyl)amino]-3-[[4-(4-morpholinyl)-1,2,5-thiadiazol-3-yl]oxy]-2-propanol (Z)-2-butene-dioate (1:1) salt.

Brevibloc (esmolol hydrochloride) is commercially available from Baxter Pharmaceutical Products Inc. and is (±)-Methyl p-[2-hydroxy-3-(isopropylamino)-propoxy]hydrocinnamate hydrochloride.

Cartrol (carteolol hydrochloride) is commercially available from Abbott and is 5-[3-[(1,1-dimethylethyl)amino]-2-hydroxypropoxy]-3,4-dihydro-2(1H)-quinolinone monohydro-chloride.

Inderal (propranolol hydrochloride) is commercially available from Wyeth-Ayerst and is 1-(Isopropylamino)-3(1-naphthyloxy)-2-propanol hydrochloride.

Kerlone (betaxolol hydrochloride) is commercially available from Searle and is 1-[4-[2-(cyclopropyl-methoxy)ethyl]phenoxy]-3-[1-methylethyl)amino]-2-propanol, hydrochloride, (±).

Levatol (Penbutolol sulfate) is commercially available from Schwarz Phanna and is (S)-1-tert-butylamino-3-(O-cyclopentylphenoxy)-2-propanol sulfate.

Lopressor (metropolol tartrate) is commercially available from Novartis and is (±)-1-(isopropylamino)-3-[p-(2-methoxyethyl)phenoxy]-2-propanol (2:1) dextro-tartrate salt.

Sectral (acebutolol hydrochloride) is commercially available from Wyeth-Ayerst Laboratories and is the hydrochloride salt ±N-[3-Acetyl-4-[2-hydroxy-3-[(1-methylethyl)-amino]propoxy]phenyl]-butanamide or (±)-3′-Acetyl-4′-[2-hydroxy-3-(isopropylamino)-propoxy]butyranilide.

Tenormin (atenolol) is commercially available from Zeneca and is 4-[2′-hydroxy-3′-[(1-methylethyl)amino]propoxy]′benzeneacetamide.

Toprol-XL (metoprolol succinate, extended release) is commercially available from Zeneca and is (±)1-(isopropylamino)-3-[p-(2-methoxyethyl) phenoxy]-2-propanol succinate (2:1) (salt).

Zebeta (bisoprolol fumurate) is commercially available from Lederle Labs and is (±)-1-[4-[[2-(1-Methylethoxy)ethoxy]methyl]phenoxy]-3-[(1-methylethyl)amino]-2-propanol (E)-2-butenedioate (2:1) (salt).

Adalat (nifedipine) is commercially available from Bayer Pharma and is 1,4-dihydro-2,6-dimethyl-4-(2-nitrophenyl)-3,5-pyridine carboxylic acid, dimethyl ester.

Adalat CC (nifedipine) is commercially available from Bayer Pharma and is 1,4-diliydro-2,6-dimethyl-4-(2-nitrophenyl)-3,5-pyridine carboxylic acid dimethyl ester.

Calan (verapamil hydrochloride) is commercially available from Searle and is α-[[3-[[2-(3,4-dimethoxyphenyl)ethyl]methyl-amino]propyl]-3,4-dimeth-oxy-α-(1-methylethyl) benzeneacetonitrile hydrochloride.

Calan SR (verapamil hydrochloride) is commercially available from Searle and is α-[[3-[[2-(3,4-dimethoxyphenyl)ethyl]methyl-amino]propyl]-3,4-dimethoxy-α-(1-methylethyl) benzeneacetonitrile hydrochloride.

Cardene (nicardipine hydrochloride) is commercially available from Wyeth-Ayerst and is 2-(benzyl-methyl amino)ethyl methyl 1,4-dihydro-2,6-dimethyl-4-(m-nitrophenyl)-3,5-pyridine-dicarboxylate monohydrochloride.

Cardizem CD (diltiazem hydrochloride) is commercially available from Hoechst Marion Roussel and is (+)-cis-1,5-benzothiazepin-4(5H)one,3-(acetyloxy)-5-[2-(dimethyl-amino)ethyl]-2,3-dihydro-2-(4-methoxyphenyl)-, monohydrochloride.

Cardizem (diltiazem hydrochloride) is commercially available from Hoechst Madon Roussel and is (+)-cis-1,5-benzothiazepin4(5H)one,3-(acetyloxy)-5-[2-(dimethyl-amino)ethyl]-2,3-dihydro-2-(4-methoxyphenyl)-monohydrochloride.

Cardizem SR (diltiazem hydrochloride) is commercially available from Hoechst Madon Roussel and is (+)-cis-1,5-benzothiazepin-4(5H)one,3-(acetyloxy)-5-[2-(dimethyl-amino)ethyl]-2,3-dihydro-2-(4-methoxyphenyl)-monohydrochloride.

Covera-HS (verapamil hydrochloride) is commercially available from Searle and is Benzeneacetonitrile, (±)-α[3[[2-(3,4-dimethoxyphenyl)ethyl]-methylamino]propyl]-3,4-dimeth-oxy-α-(1-methylethyl) hydrochloride.

Dilacor XR (dilitiazem) is commercially available from Watson and is (+)-cis-3-(acetyloxy)-5-[2-(dimethylamino)ethyl]-2,3-dihydro-2-(4-meth-oxyphenyl)-1,5-benzothiazepin-4(5H)-one monohydrochloride.

DynaCire (isradipine) is commercially available from Novartis and is 4-(4-benzofurazanyl)-1,4-dihydro-2,6-dimethyl-3,5-pyridine dicarboxylic acid, methyl-1-methyl-ethyl ester.

DynaCirc CR (isradipine) is commercially available from Novartis and is 4-(4-benzofurazanyl)-1,4-dihydro-2,6-dimethyl-3,5-Pyridine dicarboxylic acid, methyl-1-methyl-ethyl ester.

Isoptin SR (verapamil) is commercially available from Knoll AG and is (±)-α-[3[[2-(3,4-dimethoxyphenyl)ethyl]methylamino]propyl]-3,4-dimethoxy-α-(1-methylethyl)-benzeneacetonitrile hydrochloride.

Nimotop (nimodipine) is commercially available from Bayer Pharma and is isopropyl-(2-methoxyethyl)-1,4-dihydro-2,6-dimethyl-4-(3-nitrophenyl)-3,5-pyridine-di-carboxylate.

Norvasc (amlodipine besylate) is commercially available from Pfizer and is (R,S)-3-ethyl-5-methyl-2-(2-aminoethoxymethyl)-4-(2-chlorophenyl)-1,4-dihydro-6-methyl-3,5-pyridine-di-carboxylate benzenesulphonate.

Plendil (felodipine) is commercially available from Zeneca and is (±)-ethylmethyl-4-(2,3-dichlorophenyl)-1,4-dihydro-2,6-dimethyl-3,5-pyridinedicarboxylate.

Procardia (nifedipine) is commercially available from Pfizer and is 3,5-pyridine-dicarboxylic acid, 1,4-dihydro-2,6-dimethyl-4-(2-nitrophenyl)-dimethyl ester.

Procardia XL (nifedipine, extended release) is commercially available from Pfizer and is 3,5-pyridinedicarboxylic acid, 1,4-dihydro-2,6-dimethyl-4-(2-nitrophenyl)-dimethyl ester.

Sular (nisoldipine) is commercially available from Zeneca and is 3,5-pyridine-dicarboxylic acid, 1,4-dihydro-2,6-dimethyl-4-(2-nitrophenyl)-, methyl 2-methylpropyl ester.

Tiazac (diltiazem hydrochloride) is commercially available from Forest Pharmaceuticals and is (+)-cis-1,5-Benzothiazepin-4(5H)-one-3-(acetyloxy)-5[2-(dimethyl-amino)ethyl]-2,3-di-hydro-2(4-methoxyphenyl) monohydrochloride.

Vascor (bepridil hydrochloride) is commercially available from Ortho-McNeil Pharmaceuticals and is (±)-β-[(2-Methylpropoxy)methyl]-N-phenyl-N-(phenylmethyl)-1-pyrrolidine-ethanamine monohydrochloride monohydrate.

Verelan (Verapamil) is commercially available from Schwarz and is benzeneacetonitrile, a-[3-[[2-(3,4-dimethoxyphenyl)ethyl]methylamino]propyl]-3,4-dimeth-oxy-a-(1-methylethyl) monohydrochloride.

Daranide (dichlorphenamide) is commercially available from Merck and is 4,5-dichloro-1,3-benzenedisulfonamide).

Demadex (torsemide) is commercially available from Wyeth-Ayerst and is 1-iso-propyl-3-[(4-m-toluidino-3-pyridyl) sulfonyl]urea.

Edecrin (ethacrynic acid) is commercially available from Merck and is [2,3-di-chloro-4-(2-methylene-1-oxo-butyl) phenoxyl]acetic acid.

Edecrin sodium (ethacrynic acid) is commercially available from Merck and is the sodium salt of [2,3-dichloro-4-(2-methylene-1-oxo-butyl) phenoxyl]acetic acid.

Lasix (furosemide) is commercially available from Hoechst Marion Roussel and is 4-chloro-N-furfuryl-5-sulfamoylanthranilic acid.

Aldactone (Spironolactone) is commercially available from Searle and is 17-hydroxy-7α-mercapto-3-oxo-17α-pregn-4-ene-21-carboxylic acid δ-lactone acetate.

Dyrenium (triamterene) is commercially available from SmithKline Beecham and is 2,4,7-triamino-6-phenyl-pteridine.

Midamor (amiloride) is commercially available from Merck and is 3,5-diamino-6-chloro-N-(diaminomethylene) pyrazinecarboxamide monohydrochloride, dihydrate.

Diucardin (hydroflumethazide) is commercially available from Wyeth-Ayerst and is 3,4.Dihydro-6-(trifluoromethyl)-2H-1,2,4-benzothiadiazine-7 sulfonamide 1,1-dioxide.

Diuril (chlorothiazide) is commercially available from Merck and is 6-chloro-2H-1,2,4-benzothiadiazine-7-sulfonamide-1,1-dioxide.

Diuril sodium (chlorothiazide) is commercially available from Merck and is the monosodium salt of 6-chloro-2H-1,2,4-benzothiadiazine-7-sulfonamide 1,1-dioxide.

Enduron (methyclothiazide) is commercially available from Abbott and is 6-chloro-3-(chloromethyl)-3,4-dihydro-2-methyl-2H-1,2,4-benzothiadiazine-7-sulfonamide-1,1-di-oxide.

HydroDIURIL (hydrochlorothiazide (HCTZ)) is commercially available from Merck and is 6-chloro-3,4-dihydro-2H-1,2,4-benzothiadiazine-7-sulfonamide-1,1-dioxide.

Microzide (hydrochlorothiazide) is commercially available from Watson and is 6-chloro-3,4-dihydro-2H-1,2,4-benzothiadiazine-7-sulfonamide-1,1-dioxide.

Mykrox (metolazone) is commercially available from Medeva and is 7-chloro-1,2,3,4-tetra hydro-2-methyl-3-(2-methylphenyl)-4-oxo-6-quinazoline sulfonamide.

Renese (polythiazide) is commercially available from Pfizer and is 2H-1,2,4-Benzothiadiazine-7-sulfonamide, 6-chloro-3,4-dihydro-2-methyl-3-[[(2,2,2-trifluoroethyl)-thio]methyl]-1,1-dioxide.

Thalitone (chlorthalidone USP) is commercially available from Monarch and is a racemic mixture of 2-chloro-5-(1-hydroxy-3-oxo-1-isoindolinyl) benzenesulfonamide.

Zaroxolyn (metolazone) is commercially available from Medeva and is 7-chloro-1,2,3,4-tetrahydro-2-methyl-3-(2-methylphenyl)-4-oxo-6-quinazolinesulfonamide).

Hyperstat (diazoxide) is commercially available from Schering and is 7-chloro-3-methyl-2H-1,2,4-benzothiadiazine-1,1-dioxide.

Dobutrex (dobutamine) is commercially available from Lilly and is (±)-4-[2-[[3-(4-hydroxyphenyl)-1-methylpropyl]amino]ethyl]-1,2-benzenediol hydrochloride.

Lanoxicaps (digoxin) is commercially available from Glaxo Wellcome and is (3β,5β,12β)-3-[(O-2,6-dideoxy-β-D-ribo-hexopyranosyl-(1→4)-O-2,6-dideoxy-β-D-ribo-hexopyranosyl-(1→4)-2,6-dideoxy-β-D-ribo-hexopyranosyl)oxy]-12,14-dihydroxy-card-20(22)-enolide.

Lanoxin (digoxin) is commercially available from Glaxo Wellcome and is (3β,5β,12β)-3-[(O-2,6-dideoxy-β-D-ribo-hexopyranosyl-(1→4)-O-2,6-dideoxy-β-D-ribo-hexopyranosyl)oxy]-12,14-dihydroxy-card-20(22)-enolide.

Primacor (milrinone lactate injection) is commercially available from Sanofi and is 1,6-dihydro-2-methyl-6-oxo-[3,4′bipyridine]-5-carbonitrile lactate.

Demser (metyrosine) is commercially available from Merck and is (−)-α-methyl-L-tyrosine.

Inversine (Mecamylamine HCL) is commercially available from Merck and is N, 2,3,3-tetramethylbicyclo [2.2.1] heptan-2-amine hydrochloride.

Regitine (phentolamine mesylate) is commercially available from Novartis and is 4,5-dihydro-2-[N(m-hydroxy-phenyl)-N-(p-methylphenyl) aminomethyl]-1H-imidazole 1:1 methane sulfonate.

ReoPro (abciximab) is commercially available from Lilly and is the FAB fragment of the chimeric human-murine monoclonal antibody 7E3.

Diupres (reserpine and chlorothiazide) is commercially available from Merck and is 11,17α-dimethoxy-18β-[(3,4,5-trimethoxybenzoyl)oxy]-3β,20α-yohimban-16-β-carboxylic acid methylester (reserpine) and 6-chloro-2H-1,2,4-benzothiadiazine-7-sulfonamide-1,1-dioxide (chlorothiazide).

Hydropres (reserpine and hydrochlorothiazide) is commercially available from Merck and is11,17α-dimethoxy-18β-[(3,4,5-trimethoxybenzoyl)oxy]-3β,20α-yohimban-16-β-carboxylic acid methylester and 3,4-dihydro-2H-1,2,4-benzothiadiazine-7-sulfonamide-1,1-dioxide.

Deponit (Transdermal Nitroglycerin) is commercially available from Schwarz and is 1,2,3-propanetriol trinitrate.

Dilatrate-SR (isosorbide dinitrate sustained release) is commercially available from Schwarz and is 1,4:3-6-dianhydro-D-glucitol 2,5-dinitrate.

Imdur (isosorbide mononitrate) is commercially available from Key and is 1,4:3-6-dianhydro-D-glucitol, 5-nitrate.

Ismo (isosorbide mononitrate) is commercially available from Wyeth-Ayerst and is 1,4:3-6-dianhydro-D-glucitol, 5-nitrate.

Isordil (isosorbide dinitrate) is commercially available from Wyeth-Ayerst and is 1,4:3-6-dianhydro-D-glucitol 2,5-dinitrate.

Monoket (isosorbide mononitrate) is commercially available from Schwarz Pharma and is 1,4:3-6-dianhydro-D-glucitol, 5-nitrate.

Nitro-Bid (nitroglycerin) is commercially available from Hoechst Marion Roussel and is 1,2,3-propanetriol trinitrate.

Nitro-Dur (nitroglycerin) is commercially available from Key and is 1,2,3-propanetriol trinitrate.

Nitrolingual (nitroglycerin in propellants) is commercially available from Rhone-Poulenc Rorer and is 1,2,3-propanetriol trinitrate.

Nitrostat (nitroglycerin) is commercially available from Parke-Davis and is 1,2,3-propanetriol trinitrate.

Sorbitrate (isosorbide dinitrate) is commercially available from Zeneca and is 1,4:3,6-dianhydro-D-glucitol-2,5-dinitrate.

Transderm-Nitro (nitroglycerin) is commercially available from Novartis and is 1,2,3-propanetriol trinitrate.

Corlopam (fenoldopam mesylate) is commercially available from Neurex and is 6-chloro-2,3,4,5-tetrahydro-1-(4-hydroxyphenyl)-[1H]-3-benzazepine-7,8-diol methanesulfonate.

Flolan (epoprostenol sodium) is commercially available from Glaxo Wellcome and is (5Z,9α,11α,13E,15S)-6,9-epoxy-11,15-dihydroxyprosta-5,13-dien-1-oic acid.

Primacor (milrinone lactate injection) is commercially available from Sanofi and is 1,6-dihydro-2-methyl-6-oxo-[3,4′-bipyridine]-5-carbonitrile lactate.

Ana-Kit (epinephrine) is commercially available from Bayer and is 1-(3,4-dihydroxyphenyl)-2-(methylamino) ethanol.

Aramine (Metaraminol bitartrate) is commercially available from Merck and is [R-(R*,S*)]-α-(1-aminoethyl)-3-hydroxybenzenemethanol-[R-(R*,R*)]-2,3-dihydroxy-butanedioate (1:1) (salt).

EpiPen (epinephrine) is commercially available from Dey Products and is 1-(3,4-di-hydroxyphenyl)-2-(methylamino)ethanol.

ProAmatine (midodrine hydrochloride) is commercially available from Roberts and is (±)-2-amino-N-[2-(2,5-dimethoxyphenyl)-2-hydroxyethyl]acetamide monohydrochloride or (±)-2-amino-N-(β-hydroxy-2,5-dimethoxyphenethyl)acetamide monohydrochloride BAN, INN, JAN.

Vasoxyl (methoxamine hydrochloride) is commercially available from Glaxo Wellcome and is α-(1-aminoethyl)-2,5-dimethoxybenzenemethanol hydrochloride.

It is appreciated that those skilled in the art understand that the cardiovascular agent useful in the present invention is the biologically active compound present in any of the cardiovascular compositions disclosed above. For example, Cardizem (diltiazem HCL) is typically available as an injectable, as a sustained release capsule and as a direct compression tablet. The cardiovascular agent, however, is (+)-cis-1,5-benzothiazepin-4(5H)one,3-(acetyloxy)-5-[2-(dimethyl-amino)ethyl]-2,3-dihydro-2-(4-methoxyphenyl)-monohydrochloride,. Physician's Desk Reference (PDR), Medical Economics Company (Montvale, N.J.), (53rd Ed.), pp. 1311-1318, 1999.

Vitamin B₁₂

For several years after the isolation of vitamin B₁₂ as cyanocobalamin in 1948, it was assumed that cyanocobalamin and possibly hydroxocobalamin, its photolytic product, occurred in man. Later, researchers recognized that cyanocobalamin is an artifact of the isolation of vitamin B₁₂ and that hydroxocobalamin and the its coenzyme forms, methylcobalamin and adenosylcobalamin, are the naturally occurring forms of the vitamin.

The structure of these various forms is shown in FIG. 1, wherein X is CN, OH, CH₃ or adenosyl, respectively. Hereinafter, the term cobalamin will be used to refer to the molecule in its entirety, except the X group. The fundamental ring system without cobalt (Co) or side chains is called corrin and the octadehydrocorrin is called corrole. FIG. 1 is adapted from The Merck Index, Merck & Co. (11th ed. 1989), wherein X is above the plane defined by the corrin ring and nucleotide is below the plane of the ring. The corrin ring has attached six amidoalkyl (H₂NC(O)Alk) substituents, at the 2, 3, 7, 8, 13 and 18 positions, which can be designated a-e and g, respectively. See D. L. Anton et al., J. Amer. Chem. Soc., 102, 2215 (1980). Methylcobalamin serves as the cytoplasmic coenzyme for 5N-methyltetrahydrofolate: homo-cysteine methyl transferase (methionine synthetase, EC 2.1.1.13), which catalyzes the formation of methionine from homocysteine. Adenosylcobalamin is the mitochondrial coenzyme for methylmalonyl CoA mutase (EC5.4.99.2) which interconverts methylmalonyl CoA and succinyl CoA.

Vitamin B₁₂ is water soluble, has no known toxicity and, in excess, is excreted by glomerular filtration. However, Vitamin B₁₂ alone is not effective in treating or preventing any of the forms of heart disease. Accordingly, additional compounds are needed, that are suitable in treating or preventing heart disease (e.g. arteriosclerosis). Such compounds will preferably localize in or near the lining of cardiovascular vessels, especially those vessels containing plaque.

T. M. Houts (U.S. Pat. No. 4,465,775) reported that the components of the radiolabeled mixture of Niswender et al. did not bind with equal affinity to IF. Houts disclosed that radioiodinated derivatives of the pure monocarboxylic (d)-isomer are useful in assays of B₁₂ in which IF is used.

PCT Publication WO 98/08859 discloses bioconjugates (i.e. conjugates containing a bioactive agent and an organocobalt complex in which the bioactive agent is covalently bound directly or indirectly, via a spacer, to the cobalt atom). The organocobalt complex can be cobalamin and the bioactive agent can be a chemotherapeutic agent. However, only one bioactive agent (i.e. chemotherapeutic agent) is attached to the organocobalt complex (i.e. cobalamin) and the attachment is to the cobalt atom (i.e. the 6-position of cobalamin). The bioactive agent is released from the bioconjugate by the cleavage of the weak covalent bond between the bioactive agent and the cobalt atom as a result of normal displacement by cellular nucleophiles or enzymatic action or by application of an external signal (e.g. light, photoexcitation, ultrasound or the presence of a magnetic field).

PCT Publication WO 97/18231 discloses radionuclide labeling of vitamin B₁₂ through the propionamide moieties on naturally occurring vitamin B₁₂. In WO 97/18231, the inventors converted the propionamide moieties at the b-, d- and e-positions of the corrole ring to monocarboxylic acids, through a mild hydrolysis and separated the carboxylic acids by column chromatography. The inventors then attached a bifunctional linking moiety to the carboxylate function through an amide linkage and a chelating agent to the linking moiety again through an amide linkage. The chelating moiety was then used to attach an imaging radionuclide to the vitamin.

U.S. Pat. No. 5,428,023 to Russell-Jones et al. discloses a vitamin B₁₂ conjugate for delivering oral hormone formulations. The hormones are attached to the vitamin B₁₂ through a hydrolyzed propionamide linkage on the vitamin. The Patent states that the method is useful for orally administering hormones, bioactive peptides, therapeutic agents, antigens and haptens and lists as therapeutic agents neomycin, salbutamol cloridine, pyrimethamine, penicillin G, methicillin, carbenicillin, pethidine, xylazine, ketamine hydrochloride, mephanesin and iron dextran. U.S. Pat. No. 5,548,064 to Russell-Jones et al. discloses a vitamin B₁₂ conjugate for delivering erythropoietin and granulocyte-colony stimulating factor, using the same approach as the '023 Patent.

PCT Publication WO 94/27641 to Russell-Jones et aL discloses a vitamin B₁₂-polymeric linker system for the oral delivery of various active agents. In particular, WO 94/27641 discloses the attachment of various polymeric linkers to the propionamide positions of the vitamin B₁₂ molecule and the attachment of various bioactive agents to the polymeric linker. Exemplary bioactive agents include hormones, bioactive peptides and polypeptides, antitumor agents, antibiotics, antipyretics, analgesics, antiinflammatories and haemostatic agents. Exemplary polymers include carbohydrates and branched chain amino acid polymers. The linkers used in WO 94/27641 were all extremely large (each having a molecular weight of about 5000 or greater). Moreover, the linkers were of uncertain length, due to the polymerization process by which they were made.

PCT Publication WO 99/65930 to Russell-Jones et al. discloses the attachment of various agents to the 5′OH position on the vitamin B₁₂ ribose ring. The publication indicates that the system can be used to attach polymers, nanoparticles, therapeutic agents, proteins and peptides to the vitamin.

U.S. Pat. No. 5,574,018 to Habberfield et al. discloses conjugates of vitamin B₁₂ in which a therapeutically useful protein is attached to the primary hydroxyl site of the ribose moiety. The Patent lists erythropoietin, granulocyte-colony stimulating factor and human intrinsic factor as therapeutically useful proteins and indicates that the conjugates are particularly well adapted for oral administration.

U.S. Pat. No. 5,840,880 to Morgan, Jr. et al. discloses vitamin B₁₂ conjugates to which are linked receptor modulating agents, which affect receptor trafficking pathways that govern the cellular uptake and metabolism of vitamin B₁₂. The receptor modulating agents are linked to the vitamin at the b-, d- or e-position.

### SUMMARY OF THE INVENTION

In one embodiment, a compound is provided that includes a transcobalamin- or intrinsic factor-binding agent (also referred to herein as TC- or IF-binding agent) linked to a cardiovascular agent, or an active residue thereof, or its pharmaceutically acceptable salt or prodrug thereof. In one embodiment, the transcobalamin- or intrinsic factor-binding agent is a vitamin B₁₂ carrier that is covalently linked directly or via a spacer group to the cardiovascular agent. In an alternative embodiment, the transcobalamin- or intrinsic factor-binding agent that is covalently linked to the cardiovascular agent has the chemical structure indicated in formula I. The transcobalamin- or intrinsic factor-binding agent can be covalently linked to the cardiovascular agent via conventional chemical processes. It has been discovered that such compounds will localize in or near the cardiovascular system, especially the lining of cardiovascular vessels, especially those vessels containing plaque.

In another embodiment, cardiovascular diseases are diagnosed and or mapped by the use of a compound that includes a transcobalamin- or intrinsic factor-binding agent linked to a detectable radionuclide (e.g. metallic radioisotope or non-metallic radioisotope) or paramagnetic metal atom, or its pharmaceutically acceptable salt, which will localize in or near the lining of cardiovascular vessels, especially those vessels containing plaque. It has been discovered that a compound wherein a TC- or IF-binding agent is linked to a residue of an imaging agent or its pharmaceutically acceptable salt will localize in or near the lining of cardiovascular vessels, especially those vessels containing plaque.

In a preferred embodiment, the cardiovascular agent or imaging agent and the TC- or IF-binding agent or its pharmaceutically acceptable salt or prodrug thereof, is localized in or near the lining of cardiovascular vessels, especially those vessels containing plaque, in a manner that bypasses or at least does not rely on, the gastrointestinal route of absorption via the vitamin B₁₂ intrinsic factor binding protein. Preferred modes of administration are parenteral, intraperitoneal, intravenous, intradermal, epidural, intraspinal, intrasternal, intra-articular, intra-synovial, intrathecal, intra-arterial, intracardiac, intramuscular, intranasal, subcutaneous, intraorbital, intracapsular, topical, transdermal patch, via rectal, vaginal or urethral administration including via suppository, percutaneous, nasal spray, surgical implant, internal surgical paint, infusion pump or via catheter. In one embodiment, the agent and carrier are administered in a slow release formulation such as a direct tissue injection or bolus, implant, microparticle, microsphere, nanoparticle or nanosphere.

In an alternative embodiment, it has been discovered that an agent for the treatment of or for the imaging of a cardiovascular disorder can be highly and effectively localized in or near the lining of cardiovascular vessels, especially those vessels containing plaque, by direct or indirect attachment to a compound that binds to the intrinsic factor (IF-binding agent), wherein the IF-binding agent and cardiovascular or imaging agent are administered parenterally, for example, using any of the methods listed above.

The TC- or IF-binding agent and the cardiovascular agent or the imaging agent or its pharmaceutically acceptable salt or prodrug thereof, can be administered in the course of surgical or medical treatment of the afflicted site. For example, the TC- or IF-binding agent and active agent can be positioned directly at the site in or near the lining of cardiovascular vessels, especially those vessels containing plaque, during the course of surgery either by painting the formulation (with or without a controlled release matrix) onto the surface of the afflicted area or by depositing a bolus of material in a suitable matrix that is released into the afflicted area over time. In another embodiment, the TC- or IF-binding agent and the active agent are administered directly into or near the lining of cardiovascular vessels, especially those vessels containing plaque, via injection or catheter.

In another embodiment, the TC- or IF-binding agent and the cardiovascular agent or imaging agent is combined with either intrinsic factor or a transcobalamin carrier protein or both and administered parenterally, for example, via intravenous, intramuscular, direct injection or catheter, to the afflicted location.

The TC-or IF-binding agent and cardiovascular agent or imaging agent useful to image sites of cardiovascular disease in the body, such as plaques, can optionally be joined by means of a di- or multi-valent linking moiety. The linker used to join the TC- or IF-binding agent and the active agent preferably has a single molecular weight and does not exhibit a molecular weight distribution, for example as found in most polymers. The linker can range in size from small to large molecular weight, as long as there is not a distribution of weights in the linker. It is important to strictly control the uniformity of size of the conjugate for predictability of therapeutic performance.

The linkers preferably have a molecular weight below about 2000, more preferably below about 1900 or 1800 and even more preferably below about 1500 or 1000.

Thus, in one embodiment the invention provides a cardiovascular agent or an imaging conjugate having a high specificity for the lining of cardiovascular vessels, especially those vessels containing plaques, comprising (1) a TC- or IF-binding agent and (2) a cardiovascular or an imaging agent linked directly or through a linker to the TC- or IF-binding agent, wherein the linker has either (i) a unimodal (i.e. single) and defined molecular weight or (ii) a molecular weight less than about 2000 and preferably, below 1900, 1800 or 1500.

In one embodiment, the TC- or IF-binding agent is any moiety that will bind to a transcobalamin receptor and is able to be linked to a cardiovascular or an imaging agent. Methods for the assessment of whether a moiety binds the TC receptor are known and include those described by Pathare, et al., (1996) Bioconjugate Chem. 7, 217-232; and Pathare, et al., Bioconjugate Chem. 8, 161-172. An assay that assess binding to a mixture of transcobalamin I and II receptors is found in Chaiken, et al, Anal. Biochem. 201, 197 (1992). An unsaturated vitamin B₁₂ binding capacity (UBBC) assay to assess the in vitro binding of the conjugate to the transcobalamin proteins is described by D. A. Collins and H. P. C. Hogenkamp in J. Nuclear Medicine, 1997, 38, 717-723. See also Fairbanks, V. F. Mayo Clinical Proc. 83, Vol 58, 203-204.

In another embodiment the TC binding carrier or IF binding carrier is represented by formula I.

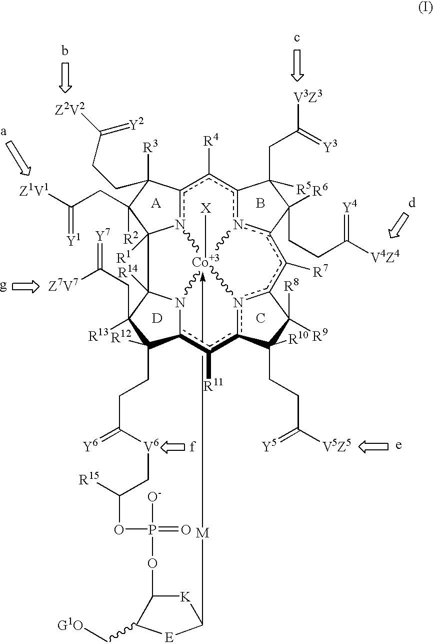

wherein:

the wavy line in the chemical structure indicates either a dative or covalent bond such that there are three dative Co—N bonds and one covalent Co—N bond, wherein, in the case of the dative bond, the valence of nitrogen is completed either with a double bond with an adjacent ring carbon or with a hydrogen;

the dotted line in the chemical structure indicates either a double or single bond such that the double bond does not over-extend the valence of the element (i.e. to give pentavalent carbons) and, in the case of a single bond, the valence is completed with hydrogen; and

wherein, in a preferred embodiment, the bonding and stereochemistry of the compound is the same as that of vitamin B₁₂ as it exists in nature.

X is hydrogen, cyano, halogen (Cl, F, Br or D, haloalkyl (including CF₃, CF₂CF₃, CH₂CF₃ and CF₂Cl), NO, NO₂, NO₃, phosphonate (including alkyl-P(O)₂OR¹⁵), PR¹⁵R¹⁶R¹⁷, NH₂, NR¹⁵R¹⁶, OH, OR¹⁵, SR¹⁵, SCN, N₃, OC(O)R¹⁵, C(O)₂R¹⁵, C(O)R¹⁵, OC(O)NR¹⁵R¹⁶, C(O)₂NR¹⁵R¹⁶, C(O)NR¹⁵R¹⁶, P(O)₂OR¹⁵, S(O)₂OR¹⁵, a purine or pyrimidine nucleoside or nucleoside analog, including adenosyl (preferably linked through a 5′-deoxy linkage) and 5-FU, alkyl, alkenyl, alkynyl, aryl, aralkyl, alkaryl, amino acid, peptide, protein, carbohydrate, heteroalkyl, heterocycle, heteroaryl or alkylheteroaryl. In one embodiment which is less preferred, X is L-T or L-T′.

M is a monovalent heterocycle or heteroaromatic, which is capable of binding to the adjacent sugar ring. M is preferably a benzimidazole, a 5- and/or 6-substituted benzimidazole, such as 5,6-dimethylbenzimidazole, 5-methyl-benzimidazole, 5-hydroxy-benzimidazole, 5-methoxy-benzimidazole, naphth-imidazole, 5-hydroxy-6-methyl-benz-imidazole or 5-methoxy-6-methyl-benz-imidazole; or a purine or pyrimidine including but not limited to adenine, 2-methyladenine, 2-methylmercaptoadenine, e-methylsulfinyl-adenine, 2-methyl-sulfonyladenine and guanine; or a phenol, such as phenol or p-cresol. The heterocycle or heteroaromatic can optionally be substituted with L-T or L-T′.

K is O, S, NJ¹, C(OH)H, CR¹⁰⁰R¹⁰¹ or C(R¹⁰⁰)V⁸Z⁸.

E is O, S, SO₂ or CH₂.

G¹ is hydrogen, alkyl, acyl, silyl, phosphate, L-T or L-T′.

Y¹, Y², Y³, Y⁴, Y⁵, Y⁶ and Y⁷ independently are O, S or NJ².

V¹, V², V³, V⁴, V⁵, V⁶, V⁷ and V⁸ independently are O, S, NJ³, CR¹⁰² R¹⁰³ or a direct bond.

Z¹, Z², Z³, Z⁴, Z⁵, Z⁷ and Z⁸ independently are R¹⁰⁴, L-T or L-T′.

Each L is independently a direct bond or a linker to one or more T or T′ moieties and that does not significantly impair the ability of the TC- or IF-binding agent to bind to a transcobalamin receptor.

Each T independently comprises a cardiovascular agent, or a pharmaceutically acceptable residue thereof, optionally bound through a chelating moiety if necessary or desired. Each T′ independently comprises an imaging agent, optionally bound through a chelating moiety if necessary or desired. In one embodiment, T is a cardiovascular agent for the treatment or prevention of cardiovascular disease. In an alternate embodiment, T′ is an imaging agent for the diagnosis of cardiovascular disease.

At least one of Z¹, Z², Z³, Z⁴, Z⁵, Z⁷, Z⁸, M and G¹ is independently L-T or L-T′. In a preferred embodiment, at least one of Z¹, Z², Z³, Z⁴, Z⁵, Z⁷, Z⁸ and G¹ is independently L-T, wherein T is independently a cardiovascular agent. In another embodiment, the compound of formula I contain at least one T that is independently a cardiovascular agent and at least one T′ that is independently an imaging agent. In a preferred embodiment, Z² comprises the sole L-T in the TC- or IF-binding agent.

J¹, J² and J³ independently are hydrogen, alkyl, alkenyl, alkynyl, alkaryl, cycloalkyl, aryl, cycloaryl, heteroalkyl, heterocycle, heteroaryl, hydroxyl, alkoxy or amine.

R¹, R², R³, R⁴, R⁵, R⁶, R⁷, R⁸, R⁹, R¹⁰, R¹¹, R¹², R¹³ and R¹⁴ independently are hydrogen, lower alkyl, lower alkenyl, lower alkynyl, lower cycloalkyl, heteroalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine.

R¹³ and R¹⁴ optionally can form a double bond.

R¹⁵, R¹⁶ and R¹⁷ are independently hydrogen, alkyl, alkenyl, alkynyl, aryl, alkaryl or aralkyl group, heteroalkyl, heterocycle or heteroaromatic.

R¹⁰⁰⁰, R¹⁰¹, R¹⁰², R¹⁰³ and R¹⁰⁴ are independently hydrogen, alkyl, alkenyl, alkynyl, aryl, acyl, heteroaromatic, heteroaryl, heteroalkyl, hydroxyl, alkoxy, cyano, azido, halogen, nitro, SO₂, SO₃, thioalkyl or amino.

In naturally occurring vitamin B₁₂, there is an α-D-5,6-dimethylbenzimidazolyl ribose 3′-phosphate which is bound through the phosphate to the B₁₂ moiety and coordinated to the cobalt ion. In a modified vitamin B₁₂ TC- or IF-binding agent, the M-sugar component is likewise in an α-D configuration, although other configurations (i.e. α-L, β-D and β-L) are possible.

One of the biologically active forms of vitamin B₁₂ has a 5′-deoxyadenosyl moiety in the X position. Vitamin B₁₂ catalysis occurs via the detachment and reattachment of the methylene radical at the 5′-deoxy position of the adenosyl moiety. In one embodiment, the selected substituent in the X position is capable of similar catalysis.

In one particular embodiment the linker used to attach the TC- or IF-binding agent and the active agent (therapeutic or imaging) is a polyamine such as spermine or spermidine.

In a particular embodiment, the TC- or IF-binding agent is linked either directly or indirectly to an anti-coagulant or any other agent that deteriorates atherosclerotic plaques.

In another embodiment X comprises the residue of 5′-deoxyadenosine.

In one embodiment, the TC- or IF-binding agent comprises one or more active agents (therapeutic or imaging) at each of one or more of the b-, d- or e-cobalamin positions, linked directly or through a linker and preferably through the b-position.

In another embodiment the TC- or IF-binding agent of the present invention comprises one or more active agents (therapeutic or imaging) at M, V⁸ or G¹.

In yet another embodiment, X is NO. NO can be administered for wound healing or other known therapeutic functions of this moiety.

In still another embodiment, the active agent (therapeutic or imaging) of the present invention comprises a radionuclide.

In yet another embodiment, the active agent (therapeutic or imaging) of the present invention does not comprise a radionuclide.

In one embodiment, the compound of formula I can be understood to exclude compounds (and therapeutic methods using such compounds) in which:

X is cyano, hydroxyl, methyl, adenosine or L-T,

M is the residue of 5,6-dimethylbenzimidazole,

E is O,

K is C(OH)H,

G¹ is hydrogen,

Y¹, Y², Y³, Y⁴ , Y⁵, Y⁶ and Y⁷ are O,

L is a direct bond or a multivalent linker derived from a dicarboxylic acid (C(O)OH-alkylene-C(O)OH), a diamine (NH₂-alkylene-NH₂), an amino-carboxylic acid (C(O)OH-alkylene-NH₂), an amino acid, a peptide or a polymer of one or amino acids,

J¹, J² and J³ are all hydrogen,

all of R¹, R², R⁴, R⁵, R⁸, R⁹, R¹¹, R¹² and R¹⁵ are methyl and all of R³, R⁶, R⁷, R¹⁰, R¹³ and R¹⁴ are hydrogen, and/or

V¹Z¹, V³Z³, V⁶Z⁶ and V⁷Z⁷ are amino.

The invention also provides intermediates disclosed herein that are useful in the preparation of compounds of the present invention as well as synthetic methods for preparing the compounds of the invention.

The invention also provides a pharmaceutical composition comprising a compound of the invention, or its pharmaceutically acceptable salt or prodrug therein, and a pharmaceutically acceptable carrier or diluent.

The present invention also provides a method of preventing or treating a cardiovascular disease in a host, preferably, an animal, and even more preferably a human, comprising administering to the host a therapeutic amount of a TC- or IF-binding agent, or its pharmaceutically acceptable salt or prodrug therein, which comprises a cardiovascular agent.

The present invention also provides a method of preventing, treating and/or imaging a cardiovascular disease in a host, preferably, an animal, and even more preferably a human, comprising administering to the animal an effective amount of a TC- or IF-binding agent, or its pharmaceutically acceptable salt or prodrug therein, which comprises a cardiovascular agent and/or an imaging agent, and optionally detecting the presence of the compound.

The present invention also provides a method of imaging a cardiovascular disease in a host, preferably, an animal, and even more preferably a human, comprising administering to the animal a detectable amount of a TC- or IF-binding agent, or its pharmaceutically acceptable salt therein, which comprises an imaging agent and detecting the presence of the compound.

The invention also provides a method of preventing or treating a cardiovascular disease in a host, preferably, an animal, and even more preferably a human, comprising administering to the host a therapeutic amount of a pharmaceutical composition comprising a TC- or IF-binding agent linked to a cardiovascular agent, or its pharmaceutically acceptable salt or prodrug therein, and a pharmaceutically acceptable carrier.

The invention also provides a method of preventing, treating and/or imaging a cardiovascular disease in a host, preferably, an animal, and even more preferably a human, comprising administering to the host an effective amount of a pharmaceutical composition comprising a TC- or IF-binding agent linked to a cardiovascular agent and or an imaging agent, or its pharmaceutically acceptable salt or prodrug therein, and a pharmaceutically acceptable carrier, and optionally detecting the presence of the compound.

The invention also provides a method of imaging a cardiovascular disease in a host, preferably, an animal, and even more preferably a human, comprising administering to the host a detectable amount of a pharmaceutical composition comprising a TC- or IF-binding agent linked to an imaging agent, or its pharmaceutically acceptable salt or prodrug therein, and a pharmaceutically acceptable carrier, and detecting the presence of the compound.

The present invention also provides a method of preventing, treating and/or imaging a cardiovascular disorder in a host, preferably, an animal, and even more preferably a human, comprising administering to the host an effective amount of the TC- or IF-binding agent of the present invention linked to a cardiovascular and/or imaging agent, with another cardiovascular agent.

The invention also provides a compound of the present invention for use in medical therapy.

The invention also provides the use of a TC- or IF-binding agent linked to a cardiovascular agent, or its pharmaceutically acceptable salt or prodrug therein, for the treatment or prophylaxis of a cardiovascular disease in a host (e.g. an animal, preferably a human).

The invention also provides the use of a TC- or IF-binding agent linked to a cardiovascular agent and/or an imaging agent, or its pharmaceutically acceptable salt or prodrug therein, for the treatment, prophylaxis or diagnosis of a cardiovascular disease in a host (e.g. an animal, preferably a human).

The invention also provides the use of a TC- or IF-binding agent linked to an imaging agent, or its pharmaceutically acceptable salt or prodrug therein, for the diagnosis of a cardiovascular disease in a host (e.g. an animal, preferably a human).

The invention also provides the use of a TC- or IF-binding agent linked to a cardiovascular agent, or its pharmaceutically acceptable salt or prodrug therein, in the manufacture of a medicament for the treatment or prophylaxis of a cardiovascular disease in a host (e.g. an animal, preferably a human).

The invention also provides the use of a TC- or IF-binding agent linked to a cardiovascular agent and/or an imaging agent, or its pharmaceutically acceptable salt or prodrug therein, in the manufacture of a medicament for the treatment, prophylaxis or diagnosis of a cardiovascular disease in a host (e.g. an animal, preferably a human).

The invention also provides the use of a TC- or IF-binding agent linked to an imaging agent, or its pharmaceutically acceptable salt or prodrug therein, in the manufacture of a medicament for the diagnosis of a cardiovascular disease in a host (e.g. an animal, preferably a human).

### BRIEF DESCRIPTION OF THE FIGURES

FIG. 1 depicts the structure of cobalamin wherein X is CN (cyano), OH, CH₃ or adenosyl.

FIG. 2 illustrates a proposed synthesis of cyanocobalamin-leucine-cardiovascular agent conjugates.

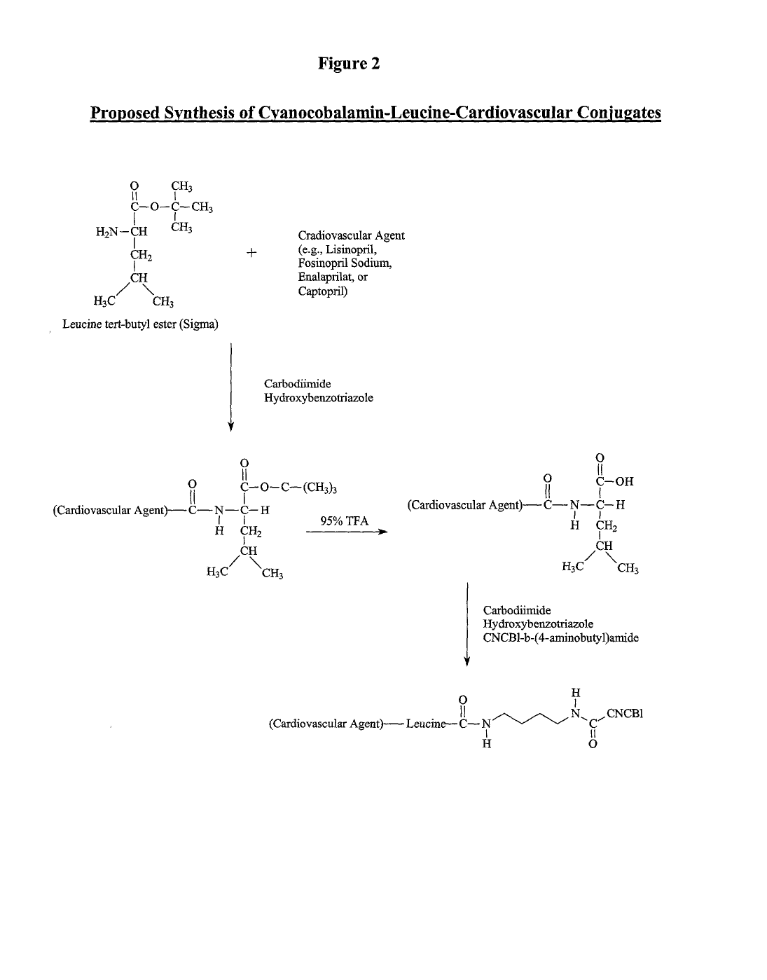

### DETAILED DESCRIPTION OF THE INVENTION

In one embodiment, a compound is provided that includes a transcobalamin- or intrinsic factor-binding agent (also referred to herein as TC- or IF-binding agent) linked to a cardiovascular agent, or an active residue thereof, or its pharmaceutically acceptable salt or prodrug thereof. In one embodiment, the transcobalamin- or intrinsic factor-binding agent is a vitamin B₁₂ carrier that is covalently linked directly or via a spacer group to the cardiovascular agent. In an alternative embodiment, the transcobalamin- or intrinsic factor-binding agent that is covalently linked to the cardiovascular agent has the chemical structure indicated in formula I. The transcobalamin- or intrinsic factor-binding agent can be covalently linked to the cardiovascular agent via conventional chemical processes. It has been discovered that such compounds will localize in or near the cardiovascular system, especially the lining of cardiovascular vessels, especially those vessels containing plaque.

In another embodiment, cardiovascular diseases are diagnosed and or mapped by the use of a compound that includes a transcobalamin- or intrinsic factor-binding agent linked to a detectable radionuclide (e.g. metallic radioisotope or non-metallic radioisotope) or paramagnetic metal atom, or its pharmaceutically acceptable salt, which will localize in or near the lining of cardiovascular vessels, especially those vessels containing plaque. It has been discovered that a compound wherein a TC- or IF-binding agent is linked to a residue of an imaging agent or its pharmaceutically acceptable salt will localize in or near the lining of cardiovascular vessels, especially those vessels containing plaque.

In a preferred embodiment, the cardiovascular agent or imaging agent and the TC- or IF-binding agent or its pharmaceutically acceptable salt or prodrug thereof, is localized in or near the lining of cardiovascular vessels, especially those vessels containing plaque, in a manner that bypasses or at least does not rely on, the gastrointestinal route of absorption via the vitamin B₁₂ intrinsic factor binding protein. Importantly, it has been discovered that oral administration of the TC- or IF-binding agent/cardiovascular agent (therapeutic or imaging) provides insufficient bioavailability to treat cardiovascular disorders. It is known that the ileal receptor for intrinsic factor-bound cobalamin is present in the gastrointestinal tract in only very small quantities and on oral delivery of vitamin B₁₂ into the alimentary system the ileal receptor can only absorb approximately two micrograms per day of vitamin B₁₂ for systemic delivery. Even assuming a small amount of systemic absorption via passive transport of a large oral dose, this level of administration is insufficient for the treatment of a cardiovascular disorder. It is important and perhaps essential, to administer the cardiovascular agent in a manner that does not rely on the ileal intrinsic factor receptor binding absorption pathway of the active agent for increased effectiveness of the agent and, in the case of imaging, decreased exposure of normal cells to the imaging agent. Preferred modes of administration are parenteral, intraperitoneal, intravenous, intradermal, epidural, intraspinal, intrastemal, intra-articular, intra-synovial, intrathecal, intra-arterial, intracardiac, intramuscular, intranasal, subcutaneous, intraorbital, intracapsular, topical, transdermal patch, via rectal, vaginal or urethral administration including via suppository, percutaneous, nasal spray, surgical implant, internal surgical paint, infusion pump or via catheter. In one embodiment, the agent and carrier are administered in a slow release formulation such as a direct tissue injection or bolus, implant, microparticle, microsphere, nanoparticle or nanosphere.

The TC- or IF-binding agent and the cardiovascular agent or the imaging agent or its pharmaceutically acceptable salt or prodrug thereof, can be administered in the course of surgical or medical treatment of the afflicted site. For example, the TC- or IF-binding agent and active agent can be positioned directly at the site in or near the lining of cardiovascular vessels, especially those vessels containing plaque, during the course of surgery either by painting the formulation (with or without a controlled release matrix) onto the surface of the afflicted area or by depositing a bolus of material in a suitable matrix that is released into the afflicted area over time. In another embodiment, the TC- or IF-binding agent and the active agent are administered directly into or near the lining of cardiovascular vessels, especially those vessels containing plaque, via injection or catheter.

In another embodiment, the TC- or IF-binding agent and the cardiovascular agent or imaging agent is combined with either intrinsic factor or a transcobalamin carrier protein or both and administered parenterally, for example, via intravenous, intramuscular, direct injection or catheter, to the afflicted location.

The TC-or IF-binding agent and cardiovascular agent or imaging agent useful to image sites of cardiovascular disease in the body, such as plaques, can optionally be joined by means of a di- or multi-valent linking moiety. The linker used to join the TC- or IF-binding agent and the active agent preferably has a single molecular weight and does not exhibit a molecular weight distribution, for example as found in most polymers. The linker can range in size from small to large molecular weight, as long as there is not a distribution of weights in the linker. It is important to strictly control the uniformity of size of the conjugate for predictability of therapeutic performance.

The linkers preferably have a molecular weight below about 2000, more preferably below about 1900 or 1800 and even more preferably below about 1500 or 1000.

Thus, in one embodiment the invention provides a cardiovascular agent or an imaging conjugate having a high specificity for the lining of cardiovascular vessels, especially those vessels containing plaques, comprising (1) a TC- or IF-binding agent and (2) a cardiovascular or an imaging agent linked directly or through a linker to the TC- or IF-binding agent, wherein the linker has either (i) a unimodal (i.e. single) and defined molecular weight or (ii) a molecular weight less than about 2000 and preferably, below 1900, 1800 or 1500.

In one embodiment, the TC- or IF-binding agent is any moiety that will bind to a transcobalamin receptor and is able to be linked to a cardiovascular or an imaging agent. Methods for the assessment of whether a moiety binds the TC receptor are known and include those described by Pathare, et al., (1996) Bioconjugate Chem. 7, 217-232; and Pathare, et al., Bioconjugate Chem. 8, 161-172. An assay that assess binding to a mixture of transcobalamin I and II receptors is found in Chaiken, et al, Anal. Biochem. 201, 197 (1992). An unsaturated vitamin B₁₂ binding capacity (UBBC) assay to assess the in vitro binding of the conjugate to the transcobalamin proteins is described by D. A. Collins and H. P. C. Hogenkamp in J. Nuclear Medicine, 1997, 38, 717-723. See also Fairbanks, V. F. Mayo Clinical Proc. 83, Vol 58, 203-204.

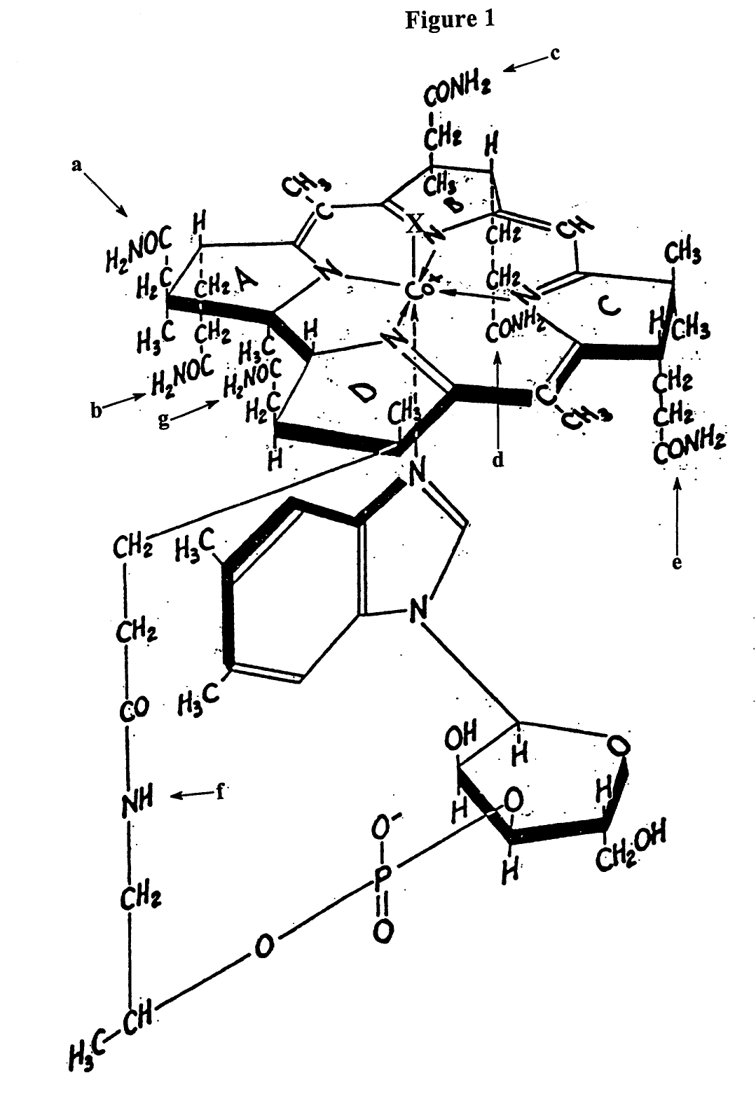

The imaging agent is preferably bound directly or indirectly through an amide residue at the b-position, as illustrated in FIG. 1.

In one embodiment, the agent and carrier are administered in a slow release formulation such as an implant, bolus, microparticle, microsphere, nanoparticle or nanosphere. Nonlimiting examples of sustained release compositions include semi-permeable polymer matrices in the form of shaped articles, e.g. films, microcapsules or microspheres. Sustained release matrices include, for example, polylactides (U.S. Pat. No. 3,773,919), copolymers of L-glutamic acid and γ-ethyl-L-glutamate (Sidman et al., Biopolymers 22:547-556, 1983) or poly-D-(-)-3-hydroxybutyric acid (EP 133,988). Sustained release compositions also include one or more liposomally entrapped compounds of formula I. Such compositions are prepared by methods known per se, e.g. as taught by Epstein et al. Proc. Natl. Acad. Sci. USA 82:3688-3692, 1985. Ordinarily, the liposomes are of the small (200-800 Å) unilamellar type in which the lipid content is greater than about 30 mol % cholesterol, the selected proportion being adjusted for the optimal therapy.

A number of sustained-release implants are known in the art. Most implants are “matrix” type and comprise an active compound dispersed in a matrix of a carrier material. The carrier material may be either porous or non-porous, solid or semi-solid and permeable or impermeable to the active compound. Matrix devices are typically biodegradable, i.e. they slowly erode after administration. Alternatively, matrix devices may be nondegradable and rely on diffusion of the active compound through the walls or pores of the matrix. Matrix devices are preferred for the applications contemplated herein.

Thus, in one embodiment the invention provides a surgical implant for localized delivery of an active agent comprising the cobalarnin conjugate of the present invention and a biodegradable binder. The implant preferably is capable of releasing and delivering the cobalamin conjugate to substantially all of an area of clear margin that results from a surgical resection and is also preferably capable of releasing the cobalamin conjugate at a substantially constant rate. In another embodiment the invention provides a method of delivering an imaging agent to an area of clear margin following a surgical resection comprising (i) providing an implant comprising a TC- or IF-binding agent linked to an imaging agent and a biodegradable binder; and (ii) placing the implant into a void created by surgical resection.

The surgical implant can exhibit a variety of forms. In one embodiment the implant is a bolus, comprising a viscous and deformable material capable of being shaped and sized before or during implantation to complement a void created by a surgical resection and sufficiently deformable upon implantation to contact substantially all of an area of clear margin. The surgical implant can also comprising a plurality of capsules that can be poured into the void created by a surgical resection. These capsules will contain the cobalamin conjugate and a suitable binder. Because they are flowable, they can be poured into the void created by a surgical lumpectomy and thereby contact substantially all of the area of clear margin.

Many suitable compositions for the implant are known and can be used in practicing the invention. Such compositions are described in, for example, Chasin et al, Biodegradable Polymers as Drug Delivery Systems, Marcel Dekker Inc., NY, ISBN 0-8247-8344-1. Preferable compositions are pharmaceutically acceptable, biodegradable and meet the particular release profile characteristics that are required to achieve the administration regime involved.

The implant typically comprises a base composition which acts as a matrix to contain and hold the contents of the implant together. The base composition can, in turn, comprise one or more constituents. Examples of base compositions include polymers and copolymers of anhydrides, orthoester, lactic acid, glycolic acid, dioxonane, trimethylene carbonate, c-caprolactone, phosphazene and glyceryl monostearate.

In one embodiment the base composition for the matrix comprises a polyanhydride, which can be synthesized via the dehydration of diacid molecules by melt condensation. Degradation times can be adjusted from days to years according to the hydrophobicity of the monomer selected. The materials degrade primarily by surface erosion and possess excellent in vivo compatibility. In one embodiment the polyanhydride is formed from sebasic acid and hexadecandioic acid (poly(SA-HDA anhydride). Wafer-like implants using this base composition have been approved for use in brain cancer, as Giadel®, by Guilford Pharmaceuticals.

The implant optionally can comprise erosion and biodegradation enhancers that facilitate the erosion of the matrix, the dissolution of the core composition or the uptake of the core composition via metabolic processes. Particularly suitable erosion and biodegradation enhancers are biodegradable in biological fluids and biocompatible. Hydrophilic constituents are typical, because they are capable of enhancing the erosion of the implant in the presence of biological fluids. For example, K. Juni et al., Chem. Pharm. Bull., 33, 1609 (1985) disclose that the release rate of bleomycin from polylactic acid microspheres is greatly enhanced by incorporating fatty acid esters into the microspheres. Other exemplary hydrophilic constituents are described, for example, in Wade & Weller, Handbook of pharmaceutical Excipients (London: Pharmaceutical Press; Washington D.C.: American Pharmaceutical Ass′n 1995) and include the polyethylene glycols (“PEGs”), propylene glycol (“PG”), glycerin and sorbitol.

Surfactants further enhance the erosion of the matrix and the release of the drug. Surfactants are generally capable of increasing the wettability and the solubility of the base composition in biological fluids and thereby causing the disintegration and erosion of the implant. Surfactants can also help to break down the core composition matrix when, for example, the method of forming the dosage form has reduced the solubility of any of the constituents. Surfactants can also improve the uptake of the dosage forms into the bloodstream. Suitable surfactants include, for example, glyceryl based surfactants such as glyceryl monooleate and glyceryl monolaurate, poloxamers such as Pluronic F127 and polysorbates such as polyoxyethylene sorbitan monooleate (“Tween 80”).

The implant could also include components that retard the rate at which the implant erodes or biodegrades (erosion and/or biodegradation retardants). Hydrophobic constituents are a particularly suitable class of components for retarding the rate at which the outer layer biodegrades. Suitable hydrophobic constituents are described, for example, in the Handbook of Pharmaceutical Excipients, the disclosure from which being hereby incorporated by reference. Exemplary hydrophobic constituents include peanut oil, olive oil and castor oil.

Any proportions or types of constituents can be chosen that effectively achieve a desired release profile and thereby carry out the prescribed administration regime. The most desirable base compositions generally release the drug substantially continuously and biodegrade completely shortly after substantially all of the drug has been effectively released. The amount of drug included in the dosage forms is determined by the total amount of the drug to be administered and the rate at which the drug is to be delivered. The total amount of the drug to be delivered is determined according to clinical requirements and in keeping with the considerations that typically inform drug dosage determinations in other contexts. The surgical implant also can contain one or more other drugs having therapeutic efficacy in the intended applications, such as a cardiovascular agent, antibiotic, an analgesic or an anesthetic.

In one embodiment, the cardiovascular agent does not include a radionuclide.

In yet another embodiment, a TC- or IF-binding agent attached to a radiodiagnostic can be used in myocardial perfusion imaging or myocardial infarction detection, to identify coronary artery disease (CAD), to detect myocardial infarction, to evaluate myocardial perfusion and to localize and estimate the size of myocardial infarcts and contusions. In this embodiment, the TC-binding or IF-binding agent and radiodiagnostic are administered, preferably via injection, to a site circumferental to the afflicted area in the skin. The radiodiagnostic is preferentially taken up by viable or necrotic myocardial cells due to the presence of the TC-binding or IF-binding agent and then is monitored in its normal course of travel in the cardiac system.

I. Definitions

It is appreciated that those skilled in the art will recognize that compounds of the present invention having a chiral center may exist in and be isolated in optically active and racemic forms. Some compounds may exhibit polymorphism. It is to be understood that the present invention encompasses any racemic, optically-active, polymorph or stereoisomeric form or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis or by chromatographic separation using a chiral stationary phase) and how to determine activity or utility as a cardiovascular agent using tests which are well known in the art.

Compounds of the present invention having a chiral center may exist in and be isolated in optically active and racemic forms. Some compounds may exhibit polymorphism. The present invention encompasses racemic, optically-active, polymorphic or stereoisomeric form or mixtures thereof, of a compound of the invention, which possess the useful properties described herein. The optically active forms can be prepared by, for example, resolution of the racemic form by recrystallization techniques, by synthesis from optically-active starting materials, by chiral synthesis or by chromatographic separation using a chiral stationary phase or by enzymatic resolution.

Specific and preferred values listed below for radicals, substituents and ranges, are for illustration only; they do not exclude other defined values or other values within defined ranges for the radicals and substituents.

The following definitions are used, unless otherwise described: halo is fluoro, chloro, bromo or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic.

Specifically, (C₁-C₆)alkyl can be methyl, ethyl, propyl, isopropyl, butyl, iso-butyl, sec-butyl, pentyl, 3-pentyl, hexyl, heptyl, octyl, nonyl, decyl, undecyl, dodecyl, tridecyl or tetradecyl.

Specifically, (C2-C24)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, 5-hexenyl, heptenyl, octenyl, nonenyl, decenyl, undecenyl, dodecenyl, tridecenyl or tetradecenyl. Specifically, (C₂-C₂₄)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, heptynyl, octynyl, nonynyl, decynyl, undecynyl, dodecynyl, tridecynyl or tetradecynyl.

Specifically “aryl” can be phenyl, indenyl or naphthyl.

Specifically (C₃-C₈)cycloalkyl can be cyclopropyl, cyclobutyl, cyclcopentyl, cyclohexyl, cycloheptyl or cyclooctyl.

As used herein, an “amino acid” is a natural amino acid residue (e.g. Ala, Arg, Asn, Asp, Cys, Glu, Gln, Gly, His, Hyl, Hyp, Ile, Leu, Lys, Met, Phe, Pro, Ser, Thr, Trp, Tyr and Val) in D or L form, as well as unnatural amino acid (e.g. phosphoserine; phosphothreonine; phosphotyrosine; hydroxyproline; gamma-carboxyglutamate; hippuric acid; octahydro-indole-2-carboxylic acid; statine; 1,2,3,4,-tetrahydroisoquinoline-3-carboxylic acid; penicillamine; omithine; citruline; α-methyl-alanine; para-benzoylphenylalanine; phenyl-glycine; propargylglycine; sarcosine; and tert-butylglycine) residue having one or more open valences. The term also comprises natural and unnatural amino acids bearing amino protecting groups (e.g. acetyl, acyl, trifluoroacetyl or benzyloxycarbonyl), as well as natural and unnatural amino acids protected at carboxy with protecting groups (e.g. as a (C₁-C₆)alkyl, phenyl or benzyl ester or amide). Other suitable amino and carboxy protecting groups are known to those skilled in the art (See for example, T. W. Greene, Protecting Groups In Organic Synthesis: Wiley: New York, 1981; D. Voet, Biochemistry, Wiley: New York, 1990; L. Stryer, Biochemistry., (3rd Ed.), W. H. Freeman and Co.: New York, 1975; J. March, Advanced Organic Chemistry, Reactions, Mechanisms and Structure, (2nd Ed.), McGraw Hill: New York, 1977; F. Carey and R. Sundberg, Advanced Organic Chemistry, Part B: Reactions and Svnthesis, (2nd Ed.), Plenum: New York, 1977; and references cited therein). According to the invention, the amino or carboxy protecting group can also comprise a non-metallic radionuclide (e.g. Fluorine-18, Iodine-123 or Iodine-124).

As used herein, a “peptide” is a sequence of 2 to 25 amino acids (e.g. as defined hereinabove) or peptidic residues having one or more open valences. The sequence may be linear or cyclic. For example, a cyclic peptide can be prepared or may result from the formation of disulfide bridges between two cysteine residues in a sequence. A peptide can be linked through the carboxy terminus, the amino terminus or through any other convenient point of attachment, such as, for example, through the sulfur of a cysteine. Peptide derivatives can be prepared as disclosed in U.S. Pat. Nos. 4,612,302; 4,853,371; and 4,684,620. Peptide sequences specifically recited herein are written with the amino terminus on the left and the carboxy terminus on the right.

As used herein, “adenosyl” is an adenosine radical in which any synthetically feasible atom or group of atoms have been removed, thereby providing an open valence. Synthetically feasible atoms that may be removed include the hydrogen atom of the hydroxy group at the 5′ position. Accordingly, adenosyl can conveniently be attached to the 6-position of a compound of formula I via the 5′ position of adenosyl.

As used herein, the term “substantially free of” or “substantially in the absence of′refers to a composition that includes at least 85 or 90% by weight, preferably 95% to 98% by weight, and even more preferably 99% to 100% by weight, of the designated enantiomer of that TC- or IF-binding agent. In a preferred embodiment, in the methods and compounds of this invention, the compounds are substantially free their enantiomers.

Similarly, the term “isolated” refers to a composition that includes at least 85 or 90% by weight, preferably 95% to 98% by weight, and even more preferably 99% to 100% by weight, of the TC- or IF-binding agent, the remainder comprising other chemical species, including diastereomers or enantiomers.

The term “independently” is used herein to indicate that the variable that is independently applied varies independently from application to application. Thus, in a compound such as R″XYR″, wherein R″ is “independently carbon or nitrogen,” both R″ can be carbon, both R″ can be nitrogen, or one R″ can be carbon and the other R″ nitrogen.

The term host, as used herein, refers to a unicellular or multicellular organism in which a cardiovascular disease can be achieved, including cell lines and animals, and preferably a human. The term host specifically refers to diseased cells and animals, in particular, primates (including chimpanzees) and humans. In most animal applications of the present invention, the host is a human patient. Veterinary applications, in certain indications, however, are clearly anticipated by the present invention (such as chimpanzees).

The term “pharmaceutically acceptable salt or prodrug” is used throughout the specification to describe any pharmaceutically acceptable form (such as an ester, mono-, di- or tri-phosphate ester, salt of an ester or a related group) of a TC- or IF-binding carrier, which, upon administration to a patient, provides the active compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. The compounds of this invention possess activity against cardiovascular disease or are metabolized to a compound that exhibits such activity.

The term “residue” is used throughout the specification to describe any pharmaceutically acceptable form of a cardiovascular agent, which, upon administration to a patient, does not inhibit the action of the cardiovascular agent. As a non-limiting example, a pharmaceutically acceptable residue of a cardiovascular agent is one that is modified to facilitate binding to the TC- or IF-binding agent, covalently, ionically or through a chelating agent, such that the modification does not inhibit the biological action of the cardiovascular agent, in that it does not inhibit the drugs ability to modulate the cardiovascular disease. In a preferred embodiment, the residue refers to the cardiovascular agent with an open valence state such that covalent bonding to the compound is possible. This open valence state can be achieved by any means known in the art, including the methodology described herein. In a preferred embodiment, the open valence state is achieved through the removal of an atom, such as hydrogen, to activate a functional group.

II. Pharmaceutically Acceptable Salt or Prodrug Formulations

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate and a-glycerophosphate. Suitable inorganic salts may also be formed, including, sulfate, nitrate, bicarbonate and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Any of the TC- or IF-binding agents described herein can be administered as a prodrug to increase the activity, bioavailability, stability or otherwise alter the properties of the carrier. A number of prodrug ligands are known. In general, alkylation, acylation or other lipophilic modification of the mono, di or triphosphate of the G¹ substuituent on the five-membered “sugar-ring” moiety will increase the stability of the carrier. Examples of substituent groups that can replace one or more hydrogens on the phosphate moiety are alkyl, aryl, steroids, carbohydrates, including sugars, 1,2-diacylglycerol and alcohols. Many are described in R. Jones and N. Bischofberger, Antiviral Research, 27 (1995) 1-17. Any of these can be used in combination with the disclosed carriers to achieve a desired effect.

The G¹ substituent of the active carrier can also provided as a 5′-phosphoether lipid or a 5′-ether lipid, as disclosed in the following references, which are incorporated by reference herein: Kucera, L. S., N. Iyer, E. Leake, A. Raben, Modest E. K., D. L. W., and C. Piantadosi. 1990. “Novel membrane-interactive ether lipid analogs that inhibit infectious HIV-1 production and induce defective virus formation.” AIDS Res. Hum. Retro Viruses. 6:491-501; Piantadosi, C., J. Marasco C. J., S. L. Morris-Natschke, K. L. Meyer, F. Gumus, J. R. Surles, K. S. Ishaq, L. S. Kucera, N. Iyer, C. A. Wallen, S. Piantadosi, and E. J. Modest. 1991. “Synthesis and evaluation of novel ether lipid nucleoside conjugates for anti-HIV activity.” J. Med. Chem. 34:1408.1414; Hosteller, K. Y., D. D. Richman, D. A. Carson, L. M. Stuhmiller, G. M. T. van Wijk, and H. van den Bosch. 1992. “Greatly enhanced inhibition of human immunodeficiency virus type 1 replication in CEM and HT4-6C cells by 3′-deoxythymidine diphosphate dimyristoylglycerol, a lipid prodrug of 3,-deoxythymidine.” Antimicrob. Agents Chemother. 36:2025.2029; Hosetler, K. Y., L. M. Stuhmiller, H. B. Lenting, H. van den Bosch, and D. D. Riclunan, 1990. “Synthesis and antiretroviral activity of phospholipid analogs of azidothymidine and other antiviral nucleosides.” J. Biol. Chem. 265:61127.

Nonlimiting examples of U.S. Patents that disclose suitable lipophllic substituents that can be covalently incorporated into the TC- or IF-binding agent, preferably at the G¹ position of the carrier or lipophilic preparations, include U.S. Pat. No. 5,149,794 (Sep. 22, 1992, Yatvin et al.); U.S. Pat. No. 5,194,654 (Mar. 16, 1993, Hostetler et al., U.S. Pat. No. 5,223,263 (Jun. 29, 1993, Hostetler et al.); U.S. Pat. No. 5,256,641 (Oct. 26, 1993, Yatvin et al.); U.S. Pat. No. 5,411,947 (May 2, 1995, Hostetler et al.); U.S. Pat. No. 5,463,092 (Oct. 31, 1995, Hostetler et al.); U.S. Pat. No. 5,543,389 (Aug. 6, 1996, Yatvin et al.); U.S. Pat. No. 5,543,390 (Aug. 6, 1996, Yatvin et al.); U.S. Pat. No. 5,543,391 (Aug. 6, 1996, Yatvin et al.); and U.S. Pat. No. 5,554,728 (Sep. 10, 1996; Basava et al.), all of which are incorporated herein by reference. Foreign Patent applications that disclose lipophilic substituents that can be attached to the carrier of the present invention, or lipophilic preparations, include WO 89/02733, WO 90/00555, WO 91/16920, WO 91/18914, WO 93/00910, WO 94/26273, WO 96/15132, EP 0 350 287, EP 93917054.4, and WO 91/19721.

III. Nonlimiting Examples of Cardiovascular Disorders

As used herein, a “cardiovascular disease” is any abnormal condition characterized by the dysfunction of the heart or blood vessels. Some examples of cardiovascular disease are disclosed, e.g. in Yale University School of Medicine Heart Book, Chapter 23, Cardiovascular Drugs, http://www.info.med.vale.edu/library/heartbk, Apr. 16, 1999; Mosby's Medical, Nursing, & Allied Health Dictionary, (25^{th} Ed.), Williams & Walkins, Baltimore, Md., 1990.

Cardiovascular diseases include arteriosclerotic heart disease (i.e. arteriosclerosis), angina pectoris, myocardial infarction, vascular diseases (e.g. peripheral vascular disease (PVD) and aneurysms), high blood pressure, hypertension, stroke (e.g. thrombotic stroke, hemorrhagic stroke and embolic stroke, congestive heart failure, valvular disease, rheumatic heart disease, cardiac arrhythmias (e.g. atrial fibrillation, ventricular tachycardia, atrial arrhythmias, ventricular fibrillation, bradyarrhythmia and premature ventricular contractions), pericarditis, myocarditis, endocarditis and cardiomyopatheies.

The compounds of the invention can optionally be administered in conjunction with one or more known cardiovascular drugs. Suitable cardiovascular drugs are disclosed hereinabove as “cardiovascular agents.”

In cases where compounds of the invention are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, α-ketoglutarate and α-glycerophosphate. Suitable inorganic salts may also be formed, including hydrochloride, sulfate, nitrate, bicarbonate and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) sales of carboxylic acids can also be made.

The compounds of the present invention can be formulated as pharmaceutical compositions and administered to a mammalian host, such as a human patient in a variety of forms adapted to the chosen route of administration, i.e. orally or parenterally (e.g. by intravenous, intramuscular, intraperitoneal). Preferably, the compounds are administered perenterally.

The active compound may also be administered intravenously or intraperitoneally by infusion or injection. Solutions of the active compound or its salts can be prepared in water, optionally mixed with a nontoxic surfactant. Dispersions can also be prepared in Glycerol, liquid polyethylene glycols, triacetin and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, propylene glycol, liquid polyethylene glycols and the like), vegetable oils, nontoxic glyceryl esters and suitable mixtures thereof. the proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimersol and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated about, as required followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For illustration, suitable doses of a compound of the invention for use in therapy, in conjunction with neutron capture, include doses in the range of from about 0.1 μg to about 100 μg, e.g. from about 0.5 μg to about 50 μg or from about 0.5 μg to 15 μg per treatment. Suitable doses for use in therapy include doses in the range of from about 0.1 mg to about 50 g, e.g. from about 0.5 mg to about 10 g or from about 0.5 g to 2 g per treatment.

The desired dose may conveniently be presented in a single dose or as divided doses administered at appropriate intervals, for example, as two, three, four or more sub-does per day. The sub-dose itself may be further divided, e.g. into a number of discrete loosely spaced administrations.

The compound are preferably dissolved or dispersed in a nontoxic liquid vehicle, such as physiological saline or a similar aqueous vehicle, to the desired concentration. A preselected therapeutic unit dose is then administered to the test animal or human patient, by oral administration or ingestion or by parenteral administration, as by intravenous or intraperitoneal infusion or injection, to attain the desired in vivo concentration. Doses useful for treating cardiovascular diseases can be derived from those found to be effective to treat cardiovascular diseases in humans in vitro or in animal models, such as those described hereinbelow or from dosage of other vitamin B₁₂molecules, previously employed in animal therapy.

IV. TC- or IF-binding Carrier

In one embodiment, the TC- or IF-binding agent is any ligand that will bind effectively to a Vitamin B₁₂ transport protein (i.e. transcobalamin I, II or III or intrinsic factor) and which when appropriately linked to an imaging agent and bound to a transport protein, will fit into a transcobalamin receptor. Methods for the assessment of whether a moiety binds the TC receptor are known and include those described by Pathare et al., Bioconjugate Chem. 1996, 7, 217-232; and Pathare, et al., Bioconjugate Chem. 8, 161-172. An assay that assess binding to a mixture of transcobalamin I and II receptors is found in Chaiken, et al, Anal. Biochem. 1992, 201, 197. An unsaturated Vitamin B₁₂ binding capacity (UBBC) assay to assess the in vitro binding of the conjugate to the transcobalamin proteins is described by D. A. Collins and H. P. C. Hogenkamp in J. Nuclear Medicine, 1997, 38, 717-723. See also Fairbanks, V. F. Mayo Clinical Proc. 83, Vol 58, 203-204. See also Fairbanks, V. F. Mayo Clinical Proc. 83, Vol 58, 203-204. The ligand preferably displays a binding affinity to transcobalamin of at least 50% of the binding affinity displayed by vitamin B₁₂, more preferably at least 75% and even more preferably at least 90%.

In one embodiment the cobalamin conjugate of the present invention is represented by formula I or an enantiomer, diastereomer, salt or pro-drug thereof:

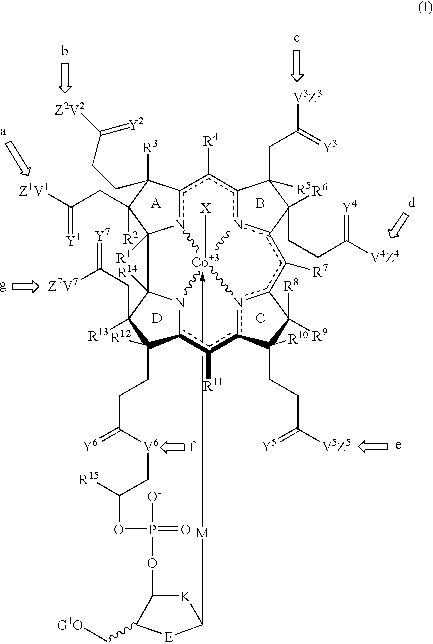

wherein:

the wavy line in the chemical structure indicates either a dative or covalent bond such that there are three dative Co—N bonds and one covalent Co—N bond, wherein, in the case of the dative bond, the valence of nitrogen is completed either with a double bond with an adjacent ring carbon or with a hydrogen;

the dotted line in the chemical structure indicates either a double or single bond such that the double bond does not over-extend the valence of the element (i.e. to give pentavalent carbons) and, in the case of a single bond, the valence is completed with hydrogen; and

wherein, in a preferred embodiment, the bonding and stereochemistry of the compound is the same as that of vitamin B₁₂ as it exists in nature.

X is hydrogen, cyano, halogen (Cl, F, Br or I), haloalkyl (including CF₃, CF₂CF₃, CH₂CF₃ and CF₂Cl), NO, NO₂, NO₃, phosphonate (including alkyl-P(O)₂OR⁵), PR¹⁵R¹⁶R¹⁷, NH₂, NR¹⁵ R¹⁶, OH, OR¹⁵, SR¹⁵, SCN, N₃, OC(O)R¹⁵, C(O)₂R¹⁵, C(O)R¹⁵, OC(O)NR¹⁵R¹⁶, C(O)₂NR¹⁵R¹⁶, C(O)NR¹⁵R¹⁶, P(O)₂OR¹⁵, S(O)₂OR¹⁵, a purine or pyrimidine nucleoside or nucleoside analog, including adenosyl (preferably linked through a 5′-deoxy linkage) and 5-FU, alkyl, alkenyl, alkynyl, aryl, aralkyl, alkaryl, amino acid, peptide, protein, carbohydrate, heteroalkyl, heterocycle, heteroaryl or alkylheteroaryl. In one embodiment which is less preferred, X is L-T or L-T′.

M is a monovalent heterocycle or heteroaromatic, which is capable of binding to the adjacent sugar ring. M is preferably a benzimidazole, a 5- and/or 6-substituted benzimidazole, such as 5,6-dimethylbenzimidazole, 5-methyl-benzimidazole, 5-hydroxy-benzimidazole, 5-methoxy-benzimidazole, naphth-imidazole, 5-hydroxy-6-methyl-benz-imidazole or 5-methoxy-6-methyl-benz-imidazole; or a purine or pyrimidine including but not limited to adenine, 2-methyladenine, 2-methylmercaptoadenine, e-methylsulfinyl-adenine, 2-methyl-sulfonyladenine and guanine; or a phenol, such as phenol or p-cresol. The heterocycle or heteroaromatic can optionally be substituted with L-T or L-T′.

K is O, S, NJ¹, C(OH)H, CR¹⁰⁰R¹⁰¹ or C(R¹⁰⁰)V⁸Z⁸.

E is O, S, SO₂ or CH₂.

G¹ is hydrogen, alkyl, acyl, silyl, phosphate, L-T or L-T′.

Y¹, Y², Y³, Y⁴, Y⁵, Y⁶ and Y⁷ independently are O, S or NJ².

V¹, V², V³, V⁴, V⁵, V⁶, V⁷ and V⁸ independently are O, S, NJ³, CR¹⁰²R¹⁰³ or a direct bond.

Z¹, Z², Z³, Z⁴, Z⁵, Z⁷ and Z⁸ independently are R¹⁰⁴, L-T or L-T′.

Each L is independently a direct bond or a linker to one or more T or T′ moieties and that does not significantly impair the ability of the TC- or IF-binding agent to bind to a transcobalamin receptor.

Each T independently comprises a cardiovascular agent, or a pharmaceutically acceptable residue thereof, optionally bound through a chelating moiety if necessary or desired. Each T′ independently comprises an imaging agent, optionally bound through a chelating moiety if necessary or desired. In one embodiment, T is a cardiovascular agent for the treatment or prevention of cardiovascular disease. In an alternate embodiment, T′ is an imaging agent for the diagnosis of cardiovascular disease.

At least one of Z¹, Z², Z³, Z⁴, Z⁵, Z⁷, Z⁸, X, M and G¹ is independently L-T or L-T′. In a preferred embodiment, at least one of Z¹, Z², Z³, Z⁴, Z⁵, Z⁷, Z⁸ and G¹ is independently L-T, wherein T is independently a cardiovascular agent. In another embodiment, the compound of formula I contain at least one T that is independently a cardiovascular agent and at least one T′ that is independently an imaging agent. In a preferred embodiment, Z² comprises the sole L-T in the TC- or IF-binding agent.

J¹, J² and J³ independently are hydrogen, alkyl, alkenyl, alkynyl, alkaryl, cycloalkyl, aryl, cycloaryl, heteroalkyl, heterocycle, heteroaryl, hydroxyl, alkoxy or amine.

R¹, R², R³, R⁴, R⁵, R⁶, R⁷, R⁸, R⁹, R¹⁰, R¹¹, R¹², R¹³ and R¹⁴ independently are hydrogen, lower alkyl, lower alkenyl, lower alkynyl, lower cycloalkyl, heteroalkyl, heterocyclic, lower alkoxy, azido, amino, lower alkylamino, halogen, thiol, SO₂, SO₃, carboxylic acid, C₁-₆ carboxyl, hydroxyl, nitro, cyano, oxime or hydrazine.

R¹³ and R¹⁴ optionally can form a double bond.

R¹⁵, R¹⁶ and R¹⁷ are independently hydrogen, alkyl, alkenyl, alkynyl, aryl, alkaryl or aralkyl group, heteroalkyl, heterocycle or heteroaromatic.

R¹⁰⁰, R¹⁰¹, R¹⁰², R¹⁰³ and R¹⁰⁴ are independently hydrogen, alkyl, alkenyl, alkynyl, aryl, acyl, heteroaromatic, heteroaryl, heteroalkyl, hydroxyl, alkoxy, cyano, azido, halogen, nitro, SO₂, SO₃, thioalkyl or amino.

In naturally occurring vitamin B₁₂, there is an a-D-5,6-dimethylbenzimidazolyl ribose 3′-phosphate which is bound through the phosphate to the B₁₂ moiety and coordinated to the cobalt ion. In a modified vitamin B₁₂ TC- or IF-binding agent, the M-sugar component is likewise in an α-D configuration, although other configurations (i.e. α-L, β-D and β-L) are possible.

One of the biologically active form of vitamin B₁₂ has a 5′-deoxyadenosyl moiety in the X position. Coenzyme B₁₂ catalysis occurs via the detachment and reattachment of the methylene radical at the 5′-deoxy position of the vitamin.

In one particular embodiment the linker used to conjugate the TC- or IF-binding agent and the imaging agent is a polyamine such as spermine or spermidine.

Because vitamin B₁₂ is preferentially taken up into the cardiovascular system, the TC- or IF-binding agent/active agent of the present invention provides a delivery system capable of targeting cardiovascular vessels, especially those vessels containing plaque and selectively imaging a greater proportion of such atherosclerotic plaques in relation to healthy vessels. A wide range of analogs and derivatives are capable of attaining these properties, as reflected by the above referenced chemical structure and variables.

The TC- or IF-binding agent can be modified in any manner that does not interfere with its fundamental ability to bind a transcobalamin transport protein and thereafter bind the TC receptor. In one embodiment, however, each variable on the vitamin B₁₂ structure independently either (i) retains its natural vitamin B₁₂ structure, (ii) imparts an imaging and/or cardiovascular agent to the cobalamin conjugate, (iii) renders the cobalamin conjugate more water soluble or more stable, (iv) increases the bioavailability of the carrier; (v) increases or at least does not decrease the binding affinity of the carrier for the TC-binding or IF-binding protein over vitamin B₁₂; or (vi) imparts another characteristic that is desired for pharmaceutical or diagnostic performance.

The imaging agent can be linked to the TC-binding or IF-binding moiety through a number of positions, including any of the V-Z moieties, the X moiety, the M moiety, the K moiety and/or the G¹ moiety, though as mentioned above at least one of Z¹, Z², Z³, Z⁴, Z⁵, Z⁷, Z⁸, M and G¹ moieties comprises an imaging agent. In one embodiment an imaging agent is linked to the TC- or IF-binding agent through Z², Z⁴, and/or Z⁵ (i.e. one or more of Z², Z⁴ and Z⁵ is L-T and T is an imaging agent). In a more particular embodiment an imaging agent is linked to the TC- or IF-binding agent through the Z² moiety (i.e. Z² is L-T and T is an imaging agent). In each of the foregoing embodiments, the Z moiety or moieties not containing an imaging agent preferably retain its natural vitamin B₁₂ configuration, in which VZ is NH₂. Alternatively, the Z moieties not containing an imaging agent may comprise a secondary or tertiary amino analog of NH₂ substituted by one or two of J¹.

In any Z¹, Z², Z³, Z⁴, Z⁵, Z⁶, Z⁷, Z⁸, X, M or G¹ moieties through which an imaging agent is linked, it will be understood that such moiety may comprise more than one imaging agent or a combination of imaging agents, i.e. each T can independently comprise the residue of one or more imaging agents bound to L through one or more chelating moieties. More specifically, in a series of embodiments, each T can comprise 1, 2, 3, 4, 5, 6, 7, 8, 9 or 10 imaging agents bound through one or more chelating moieties.

R¹, R², R³, R⁴, R⁵, R⁶, R⁷, R⁸, R⁹, R¹⁰, R¹¹, R¹² and R¹³independently represent moieties that do not interfere with binding between the compound and the transcobalamin transport protein or receptor. Vitamin B₁₂ can be modified through these moieties to modulate physical properties of the molecule, such as water solubility, stability or λₘₐₓ. Preferred groups for enhancing water solubility include heteroalkyl, amino, C₁-₆ alkylamino, C₁₋₆ alcohol, C₁-₆ carboxylic acid and SO₃⁻.

In another embodiment, one, some or all of R¹, R², R³, R¹, R⁵, R⁶, R⁷, R⁸, R⁹, R¹⁰, R¹¹, R¹² and R¹³ independently assume their natural roles in vitamin B₁₂. Thus, one, some or all of R¹, R², R⁴, R⁵, R⁸, R⁹, R¹¹, R¹² and R¹⁵ are independently methyl in one embodiment and one, some or all of R³, R⁶, R⁷, R¹⁰, R¹³ and R¹⁴ are independently hydrogen.

In another embodiment, one, some or all of Y¹, Y², Y³ Y⁴ Y⁵, Y⁶ and Y⁷ assume their natural roles in vitamin B₁₂ and are O. Similarly, in another embodiment V⁶ assumes its natural role in vitamin B₁₂ and is NH or a primary amine analog thereof substituted by J¹.

In still another embodiment, position X assumes its natural role in vitamin B₁₂, i.e. as cyano, hydroxyl, methyl or 5′-deoxyadenosyl, most preferably 5′-deoxyadenosyl.

In another embodiment M is the radical of a purine or pyrimidine base. In another embodiment M is the radical of adenosine, guanine, cytosine, uridine or thymine. In still another embodiment M is the radical of 5,6-dimethylbenzimidazole.

In still another embodiment K is CH(OH).

In yet another embodiment E is O.

In another embodiment G¹ is OH.

In still another embodiment, all constituents of the conjugate assume their natural roles in vitamin B₁₂, except for the moieties through which any imaging agents are linked. The imaging agent(s) are preferably linked to the vitamin B₁₂ structure through Z², Z⁴, and/or Z⁵ and even more preferably through the Z² moieties.

V. Linkers

As noted above, L is the residue of a linker molecule that conjugates one or more imaging agents to the TC ligand. The structure of the linker from which L is derived (in any one of the Z¹, Z², Z³, Z⁴, Z⁵, Z⁶, Z⁷, X, M or G¹ moieties) is not crucial, provided it does not significantly impair the ability of the conjugate to bind to the transcobalamin or IF transport protein or receptor. L is preferably any multivalent molecule (divalent or greater) that does not significantly impair the ability of the TC carrier to bind to the transcobalamin transport protein or receptor. The ability of vitamin B₁₂ or any other TC-binding carrier to bind to the transcobalamin transport protein or receptor is “significantly impaired” when attaching a linking moiety to the B₁₂ or TC-binding carrier lessens the affinity of the vitamin B₁₂ or the TC-binding carrier for the transcobalamin transport protein to which the vitamin B₁₂ or TC-binding arrier is most readily bound by 50% or more. The unsaturated vitamin B₁₂ binding capacity (UBBC) assay described by D. A. Collins and H. P. C. Hogenkamp in J. Nuclear Medicine, 1997, 38, 717-723 can be used to compare the relative affinities of ligands for this receptor.

In one embodiment the linker is of precise molecular weight and does not posses a molecular weight distribution. In one embodiment, the linker has a molecular weight less than about 2,500, 2,000, 1900, 1800, 1,500, 1,000 or 500.

A particularly preferred linker is one having multiple sites for conjugation to one or more imaging agents, wherein the linker has a unimodal molecular weight. Recombinant protein production techniques can be employed to obtain poly(amino acid) linkers of substantially constant molecular weight.

In one embodiment the linker is an amino acid or a polymer or peptide formed from a plurality of amino acids. The polymer or peptide can be derived from one or more amino acids. The amino acid, poly(amino acid) or peptide can link T to V through the carboxy terminus or the amino terminus. The amino acid residue, peptide residue or poly(amino acid) residue can conveniently be linked to V and T through an amide (e.g. —N(R)C(—O)— or —C(═O)N(R)—), ester (e.g. —OC(═O)— or —C(═O)O—), ether (e.g. —O—), ketone (e.g. —C(═O)—), thioether (e.g. —S—), sulfinyl (e.g. —S(O)—), sulfonyl (e.g. —S(O)₂—) or a direct (e.g. C—C bond) linkage, wherein each R is independently H or (C₁-C₁₄) alkyl.

Peptide derivatives can be prepared as disclosed in U.S. Pat. Nos. 4,612,302; 4,853,371; and 4,684,620. Peptide sequences specifically recited herein are written with the amino terminus on the left and the carboxy terminus on the right, but are meant to also include the opposite flow. Particularly suitable peptides and poly(amino acids) comprise from 2 to about 20 amino acids, from 2 to about 15 amino acids or from 2 to about 12 amino acids.

One exemplary poly(amino acid) is poly-L-lysine ((—NHCH((CH₂)₄—NH₂)CO—)ₘ—Q, wherein Q is H, (C₁-C₁₄)alkyl or a suitable carboxy protecting group and m is from 2 to about 20, from about 5 to about 15 or from about 8 to about 11. The polylysine offers multiple primary amine sites to which active agents can be readily attached. Alternatively, the linkers can be formed with multiple cysteines, to provide free thiols or multiple glutamates or aspartates, to provide free carboxyls for conjugation using suitable carbodiimides. Similarly the linker can contain multiple histidines or tyrosines for conjugation. Other exemplary poly(amino acid) linkers are poly-L-glutamic acid, poly-L-aspartic acid, poly-L-histidine, poly-L-ornithine, poly-L-serine, poly-L-threonine, poly-L-tyrosine, poly-L-lysine-L-phenylalanine or poly-L-lysine-L-tyrosine. When the linker is derived from a poly(amino acid) other than polylysine, the linker is, in a series of embodiments, prepared from 2 to about 30 amino acids, 5 to about 20 amino acids or 8 to about 15 amino acids.

In another particular embodiment L is a polyamine residue (having at least three amino moieties) of the following chemical structure: NR′(alkylene-NR′)nalkyleneNR′, wherein n is from 1 to 20, the carbon length of alkylene can vary within the n units and each R′ is independently hydrogen, lower alkyl or T. N is preferably from 1 to 10. Moreover, L preferably has a backbone along its longest length of no more than 100, 75, 50, 40, 30, 20 or 15 atoms. Exemplary polyamines from which L can be derived include spermine (H₂N(CH₂)₃NH(CH₂)₄NH(CH₂)₃NH₂), spermidine (H₂N(CH₂)₃NH(CH₂)₄NH₂), deca-methylene tetraamine and pentamethylene hexamine. These linkers are a definite size and thus provide consistent and predictable targeting by the cobalamin conjugate, in addition to multiple binding sites for the imaging agent.

In another embodiment L is a diamine represented by the formula NH₂ (CH₂)ₓNH₂, in which x is 2-20 and preferably 2-12. Thus, the linker can be prepared from 1,6-diaminohexane, 1,5-diaminopentane, 1,4-diaminobutane and 1,3-diaminopropane.

Other suitable linkers are formed from the covalent linkage of various water soluble molecules with amino acids, peptides, poly(amino acids), polyamines, polyoxyalkylenes, polyanhydrides, polyesters, polyamides, polyglycolides and diamines. Suitable water soluble molecules include, for example, polyethylene glycol and dicarboxylic monosaccharides such as glucaric acid, galactaric acid and xylaric acid.

Other suitable linkers include those represented by the formula HO(O)C(CH₂)ₓC(O)OH, in which x is 2-20 and preferably 2-12. Thus, the linker can be prepared from succinic acid, glutaric acid, adipic acid, suberic acid, sebacic acid, azelaic acid or maleic acid. Still other suitable linkers comprise carboxylic acid derivatives that yield an amide upon reaction with an amine. Such reactive groups include, by way of example, carboxylic acid halides such as acid chlorides and bromides; carboxylic acid anhydrides such as acetic anhydrides and trifluoroacetic anhydrides; esters such as p-nitrophenyl esters and N-hydroxysuccinimide esters; and imidazolides. Techniques for using such linkers are described in detail in Bodanszky, Principles of Peptide Synthesis, Springer Verlag, Berlin, 1984.

In one embodiment, the linker is modified to facilitate its conjugation either to V or T. Suitable molecules for modifying the linker include: disuccinimidyl suberate (DSS), bis(sulfosuccinimidyl) suberate (BSS), ethylene glycolbis(succinimidylsuccinate) (EGS), ethylene glycolbis(sulfosuccinimidyl-succinate) (Sulfo-EGS), p-aminophenylacetic acid, dithiobis(succinimidyl-propionate) (DSP), 3,3′-dithiobis-(sulfosuccinimidylpropionate) (DTSSP), disuccinimidyl tartarate (DST), disulfosuccinimidyl tartarate (Sulfo-DST), bis(2-(succinimidooxycarbonyloxy)-ethylene)sulfone (BSOCOES), bis(2-(sulfosuccinimidooxy-carbonyloxy)ethylene)sulfone (Sulfo-BSOCOES), dimethyl adipimidate.2HCl (DMA), dimethyl pimelimidate.2HCl (DMP) and dimethyl suberimidate.2HCl (DMS).

Biodegradable Linkers

Various degradable linkers can be used to link the TC-binding or IF-binding moiety to the active agent. The desired linkers can degrade under biological conditions such as by enzymatic cleavage or by systemic pH or temperature. Alternatively, these linkers can be induced to degrade by external manipulation such as changes in pH, temperature, ultrasound, magnetic field, radiation (i.e. UV radiation) or light.

U.S. Pat. No. 5,639,885 entitled “Redox amino acids and peptides containing them;” U.S. Pat. No. 5,637,601 entitled “Anticholinergic compounds, compositions and methods of treatment;” U.S. Pat. No. 5,624,894 entitled “Brain-enhanced delivery of neuroactive peptides by sequential metabolism;” U.S. Pat. No. 5,618,826 entitled “Anticholinergic compounds, compositions and methods of treatment;” U.S. Pat. No. 5,618,803 entitled “Targeted drug delivery via phosphonate derivatives;” U.S. Pat. No. 5,610,188 entitled “Anticholinergic compounds, compositions and methods of treatment;” U.S. Pat. No. 5,525,727 entitled “Brain-specific drug delivery;” U.S. Pat. No. 5,418,244 entitled “Anticholinergic compounds, compositions and methods of treatment;” U.S. Pat. No. 5,413,996 entitled “Targeted drug delivery via phosphonate derivatives;” U.S. Pat. No. 5,389,623 entitled “Redox carriers for brain-specific drug delivery;” U.S. Pat. No. 5,296,483 entitled “Brain-specific analogues of centrally acting amines;” U.S. Pat. No. 5,258,388 entitled “Anticholinergic compounds, compositions and methods of treatment;” U.S. Pat. No. 5,231,089 entitled “Method of improving oral bioavailability of carbamazepine;” U.S. Pat. No. 5,223,528 entitled “Anticholinergic compounds, compositions and methods of treatment;” U.S. Pat. No. 5,187,158 Brain-specific drug delivery;” U.S. Pat. No. 5,177,064 entitled “Targeted drug delivery via phosphonate derivatives;” U.S. Pat. No. 5,155,227 entitled “Compounds for site-enhanced delivery of radionuclides;” U.S. Pat. No. 5,136,038 entitled “Radiopharmaceuticals and chelating agents useful in their preparation;” U.S. Pat. No. 5,087,618 entitled “Redox carriers for brain-specific drug delivery;” U.S. Pat. No. 5,079,366 entitled “Quaternary pyridinium salts;” U.S. Pat. No. 5,053,215 entitled “NMR-assayable ligand-labeled trifluorothymidine containing composition and method for diagnosis of HSV infection;” U.S. Pat. No. 5,024,998 entitled “Phannaceutical formulations for parenteral use;” U.S. Pat. No. 5,017,618 entitled “Labile derivatives of ketone analogs of 3-substituted-1-alkylamino-2-propanols and their use as beta-adrenergic blockers;” U.S. Pat. No. 5,017,566 entitled “Redox systems for brain-targeted drug delivery;” U.S. Pat. No. 5,008,257 entitled “Brain-specific drug delivery;” U.S. Pat. No. 5,002,935 entitled “Improvements in redox systems for brain-targeted drug delivery;” U.S. Pat. No. 4,983,586 entitled “Pharmaceutical formulations for parenteral use;” U.S. Pat. No. 4,963,688 entitled “Compounds for site-enhanced delivery of radionuclides and uses thereof;” U.S. Pat. No. 4,963,682 entitled “Novel radiopharmaceuticals and chelating agents useful in their preparation;” U.S. Pat. No. 4,933,438 entitled “Brain-specific analogues of centrally acting amines;” U.S. Pat. No. 4,900,837 entitled “Brain-specific drug delivery of steroid sex hormones cleaved from pyridinium carboxylates and dihydro-pyridine carboxylate precursors;” U.S. Pat. No. 4,892,737 entitled “Composition and method for enhancing permeability of topical drugs;” U.S. Pat. No. 4,888,427 entitled “Amino acids containing dihydropyridine ring systems for site-specific delivery of peptides to the brain;” 4,880,921 entitled “Brain-specific drug delivery;” 35. 4,863,911 entitled “Method for treating male sexual dysfunction;” U.S. Pat. No. 4,829,070 entitled “Novel redox carriers for brain-specific drug delivery;” U.S. Pat. No. 4,824,850 entitled “Brain-specific drug delivery;” U.S. Pat. No. 4,801,597 entitled “Certain inositol-nicotinate ester derivatives and polyionic complexes therefore useful for treating diabetes meuitus, hyperlipidemia and lactic acidosis;” U.S. Pat. No. 4,771,059 entitled “Brain-specific analogues of centrally acting amines;” U.S. Pat. No. 4,727,079 entitled “Brain-specific dopaminergic activity involving dihydropyridine carboxamides, dihydroquinoline and isoquinoline carboxamides;” U.S. Pat. No. 4,540,564 entitled “Brain-specific drug delivery;” and U.S. Pat. No. 4,479,932 entitled “Brain-specific drug delivery” to Nicholas S. Bodor, et al., disclose several biodegradable linkers that target the brain. For example, a lipoidal form of dihydropyridine pyridinium salt redox carrier, DHC, linked to a centrally acting drug which can be reduced and biooxidized to pass through the blood brain barrier. The dihydropyridine nucleus readily and easily penetrates the blood brain barrier in increased concentrations; furthermore, the in vivo oxidation of the dihydropyridine moiety to the ionic pyridinium salts thereby prevents its elimination from the brain, while elimination from the general circulation is accelerated, resulting in a prolongedly sustained brain-specific drug activity. This dihydropyridine can be incorporated into the linkers set forth above for biodegradation.

Additionally U.S. Pat. No. 4,622,218 entitled “Testicular-specific drug delivery,” discloses linkers that can specifically deliver drugs to the testes in much the same manner and which can be used in the linkers of the present invention. The lipoidal form [D-DHC] of a dihydropyridine pyridinium salt redox carrier, e.g. 1,4-dihydrotrigonelline, penetrates the blood-testis barrier. Oxidation of the dihydropyridine carrier moiety in vivo to the ionic pyridinium salt type drug/carrier entity [D-QC]+prevents elimination thereof from the testes, while elimination from the general circulation is accelerated, resulting in significant and prolongedly sustained testicular-specific drug activity.

Margerum, et al. in U.S. Pat. No. 5,976,493 discloses the use of polychelant compounds which are degradable in vivo to release excretable fragments for diagnostic imaging which also are suitable in the linkers of the present invention. These compounds contain a linker moiety which is metabolically cleavable to release macrocyclic monochelant fragments, wherein the macrocyclic skeleton preferably has 9 to 25 ring members and a biotolerable polymer, preferably a substantially monodisperse polymer. Other suitable linkers are disclosed, for example, in Krejcarek et al. (Biochemical and Biophysical Research Communications 77: 581 (1977)) (mixed anhydrides), Hnatowich et al. (Science 220: 613 (1983))(cyclic anhydrides), United States Patent 5,637,684 to Cook, et al. (Phosphoramidate and phosphorothioamidate oligomeric compounds).

Other suitable biodegradable polymers from which the linker can be formed are the polyanhydrides and polyorthoesters, which take advantage of labile backbone linkages (see: Domb et al. Macromolecules, 22, 3200, 1989; and Heller et al. Biodegradable Polymers as Drug Delivery Systems, Dekker, NY: 1990). Other linker materials include hydrogels, such as the PEG-oligoglycolyl-acrylates disclosed in U.S. Pat. No. 5,626,863 to Hubbell et aL. Other biodegradable linkers are formed from oligoglycolic acid is a poly(a-hydroxy acid), polylactic acid, polycaprolactone, polyorthoesters, polyanhydrides and polypeptides.

Nonlimiting examples of U.S. Patents that describe controlled release formulations suitable for use as linking agents are: U.S. Pat. No. 5,356,630 to Laurencin et al. (Delivery System for Controlled Release of Bioactive Factors); ; U.S. Pat. No. 5,797,898 to Santini, Jr. et al. (Microchip Drug Delivery Devices); U.S. Pat. No. 5,874,064 to Edwards et al. (Aerodynamically Light Particles for Pulmonary Drug Delivery); U.S. Pat. No. 5,548,035 to Kim et al. (Biodegradable Copolymer as Drug Delivery Matrix Comprising Polyethyleneoxide and Aliphatic Polyester Blocks); U.S. Pat. No. 5,532,287 to Savage et al. (Radiation Cured Drug Release Controlling Membrane); U.S. Pat. No. 5,284,831 to Kahl et al. (Drug Delivery Porphyrin Composition and Methods); U.S. Pat. No. 5,741,329 to Agrawal et al. (Methods of Controlling the pH in the Vicinity of Biodegradable Inplants); U.S. Pat. No. 5,820,883 to Tice et al. (Methods for Delivering Bioactive Agents into and Through the Mucosally-Associated Lymphoid Tissues and Controlling Their Release);U.S. Pat. No. 5,955,068 to Gouin et al. (Biodegradable Polyanhydrides Derived from Dimers of Bile Acids and Use Thereof as Controlled Drug Release Systems); U.S. Pat. No. 6,001,395 to Coombes et al. (Polymeric Lamellar Substrate Particles for Drug Delivery); U.S. Pat. No. 6,013,853 to Athanasiou et al. (Continuous Release Polymeric Implant Carriers); U.S. Pat. No. 6,060,582 to Hubbell et al. (Photopolymerizable Biodegradable Hydrogels as Tissue Contacting Materials and Controlled Release Carriers); U.S. Pat. No. 6,113,943 to Okada et al. (Sustained-Release Preparation Capable of Releasing a Physiologically Active Substance); and PCT Publication No. WO 99/59548 to Oh et al. (Controlled Drug Delivery System Using the Conjugation of Drug to Biodegradable Polyester); U.S. Pat. No. 6,123,861 (Fabrication of Microchip Drug Delivery Devices); U.S. Pat. No. 6,060,082 (Polymerized Liposomes Targeted to M cells and Useful for Oral or Mucosal Drug Delivery); U.S. Pat. No. 6,041,253 (Effect of Electric Field and Ultrasound for Transdermal Drug Delivery); U.S. Pat. No. 6,018,678 (Transdermal protein delivery or measurement using low-frequency sonophoresis); U.S. Pat. No. 6,007,845 Nanoparticles And Microparticles Of Non-Linear Hydrophilic-Hydrophobic Multiblock Copolymers; U.S. Pat. No. 6,004,534 Targeted Polymerized Liposomes For Inproved Drug Delivery; U.S. Pat. No. 6,002,961 Transdermal Protein Delivery Using Low-Frequency Sonophoresis; U.S. Pat. No. 5,985,309 Preparation Of Particles For Inhalation; U.S. Pat. No. 5,947,921 Chemical And Physical Enhancers And Ultrasound For Transdermal Drug Delivery; U.S. Pat. No. 5,912,017 Multiwall Polymeric Microspheres; U.S. Pat. No. 5,911,223 Introduction Of Modifying Agents Into Skin By Electroporation; U.S. Pat. No. 5,874,064 Aerodynamically Light Particles For Pulmonary Drug Delivery; U.S. Pat. No. 5,855,913 Particles Incorporating Surfactants For Pulmonary Drug Delivery; U.S. Pat. No. 5,846,565 Controlled Local Delivery Of Chemotherapeutic Agents For Treating Solid Tumors; U.S. Pat. No. 5,837,752 Semi-Interpenetrating Polymer Networks; U.S. Pat. No. 5,814,599 Transdermal Delivery Of Encapsulated Drugs; U.S. Pat. No. 5,804,178 Implantation Of Cell-Matrix Structure Adjacent Mesentery, Omentum Or Peritoneum Tissue; U.S. Pat. No. 5,797,898 Microchip Drug Delivery Devices; U.S. Pat. No. 5,770,417 Three-Dimensional Fibrous Scaffold Containing Attached Cells For Producing Vascularized Tissue In vivo; U.S. Pat. No. 5,770,193 Preparation Of Three-Dimensional Fibrous Scaffold For Attaching Cells To Produce Vascularized Tissue In vivo; U.S. Pat. No. 5,762,904 Oral Delivery Of Vaccines Using Polymerized Liposomes; U.S. Pat. No. 5,759,830 Three-Dimensional Fibrous Scaffold Containing Attached Cells For Producing Vascularized Tissue In vivo; U.S. Pat. No. 5,749,847 Delivery Of Nucleotides Into Organisms By Electroporation; U.S. Pat. No. 5,736,372 Biodegradable Synthetic Polymeric Fibrous Matrix Containing Chondrocyte For In vivo Production Of A Cartilaginous Structure; U.S. Pat. No. 5,718,921 Microspheres Comprising Polymer And Drug Dispersed There Within; U.S. Pat. No. 5,696,175 Preparation Of Bonded Fiber Structures For Cell Implantation; U.S. Pat. No. 5,667,491 Method For Rapid Temporal Control Of Molecular Transport Across Tissue; U.S. Pat. No. 5,654,381 Functionalized Polyester Graft Copolymers; U.S. Pat. No. 5,651,986 Controlled Local Delivery Of Chemotherapeutic Agents For Treating Solid Tumors; U.S. Pat. No. 5,629,009 Delivery System For Controlled Release Of Bioactive Factors; U.S. Pat. No. 5,626,862 Controlled Local Delivery Of Chemotherapeutic Agents For Treating Solid Tumors; U.S. Pat. No. 5,593,974 Localized Oligonucleotide Therapy; U.S. Pat. No. 5,578,325 Nanoparticles And Microparticles Of Non-Linear Hydrophilic-Hydrophobic Multiblock Copolymers; U.S. Pat. No. 5,562,099 Polymeric Microparticles Containing Agents For Imaging; U.S. Pat. No. 5,545,409 Delivery System For Controlled Release Of Bioactive Factors; U.S. Pat. No. 5,543,158 Biodegradable Injectable Nanoparticles; U.S. Pat. No. 5,514,378 Biocompatible Polymer Membranes And Methods Of Preparation Of Three Dimensional Membrane Structures; U.S. Pat. No. 5,512,600 Preparation Of Bonded Fiber Structures For Cell Inplantation; U.S. Pat. No. 5,500,161 Method For Making Hydrophobic Polymeric Microparticles; U.S. Pat. No. 5,487,390 Gas-filled polymeric microbubbles for ultrasound imaging; U.S. Pat. No. 5,399,665 Biodegradable polymers for cell transplantation; U.S. Pat. No. 5,356,630 Delivery system for controlled release of bioactive factors; U.S. Pat. No. 5,330,768 Controlled drug delivery using polymer/pluronic blends; U.S. Pat. No. 5,286,763 Bioerodible polymers for drug delivery in bone; U.S. Pat. No. 5,149,543 lonically cross-linked polymeric microcapsules; U.S. Pat. No. 5,128,420 Method of making hydroxamic acid polymers from primary amide polymers; U.S. Pat. No. 5,122,367 Polyanhydride bioerodible controlled release implants for administration of stabilized growth hormone; U.S. Pat. No. 5,100,668 Controlled release systems containing heparin and growth factors; U.S. Pat. No. 5,019,379 Unsaturated polyanhydrides; U.S. Pat. No. 5,010,167 Poly(amide-and imide-co-anhydride) for biological application; .S. Patent No. 4,948,587 Ultrasound enhancement of transbuccal drug delivery; U.S. Pat. No. 4,946,929 Bioerodible articles useful as implants and prostheses having predictable degradation rates; U.S. Pat. No. 4,933,431 One step preparation of poly(amide-anhydride); U.S. Pat. No. 4,933,185 System for controlled release of biologically active compounds; U.S. Pat. No. 4,921,757 System for delayed and pulsed release of biologically active substances; U.S. Pat. No. 4,916,204 Pure polyanhydride from dicarboxylic acid and coupling agent; U.S. Pat. No. 4,906,474 Bioerodible polyanhydrides for controlled drug delivery; U.S. Pat. No. 4,900,556 System for delayed and pulsed release of biologically active substances; U.S. Pat. No. 4,898,734 Polymer composite for controlled release or membrane formation; U.S. Pat. No. 4,891,225 Bioerodible polyanhydrides for controlled drug delivery; U.S. Pat. No. 4,888,176 Controlled drug delivery high molecular weight polyanhydrides; U.S. Pat. No. 4,886,870 Bioerodible articles useful as implants and prostheses having predictable degradation rates; U.S. Pat. No. 4,863,735 Biodegradable polymeric drug delivery system with adjuvant activity; U.S. Pat. No. 4,863,611 Extracorporeal reactors containing immobilized species; U.S. Pat. No. 4,861,627 Preparation of multiwall polymeric microcapsules; U.S. Pat. No. 4,857,311 Polyanhydrides with improved hydrolytic degradation properties; U.S. Pat. No. 4,846,786 Bioreactor containing suspended, immobilized species; U.S. Pat. No. 4,806,621 Biocompatible, bioerodible, hydrophobic, implantable polyimino carbonate article; U.S. Pat. No. 4,789,724 Preparation of anhydride copolymers; U.S. Pat. No. 4,780,212 Ultrasound enhancement of membrane permeability; U.S. Pat. No. 4,779,806 Ultrasonically modulated polymeric devices for delivering compositions; U.S. Pat. No. 4,767,402 Ultrasound enhancement of transdermal drug delivery; U.S. Pat. No. 4,757,128 High molecular weight polyanhydride and preparation thereof; U.S. Pat. No. 4,657,543 Ultrasonically modulated polymeric devices for delivering compositions; U.S. Pat. No. 4,638,045 Non-peptide polyamino acid bioerodible polymers; U.S. Pat. No. 4,591,496 Process for making systems for the controlled release of macromolecules.

Nonmetallic radioisotopes can conveniently be linked to the vitamin B₁₂ structure through a residue of a peptide having the following formula:

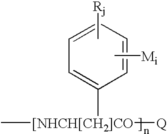

wherein each M is independently a non-metallic radionuclide; each R is independently (C₁-C₁₄)alkyl, (C₂-C₁₄)alkenyl, (C₂-C₁₄)alkynyl, (C₁-C₁₄)alkoxy, hydroxy, cyano, nitro, halo, trifluoromethyl, N(Ra)(Rb), (C₁-C₁₄)alkanoyl, (C₂-C₁₄)alkanoyloxy, (C₆-C₁₀)aryl or (C₃-C₈)cycloalkyl wherein Ra and Rb are each independently H or (C₁-C₁₄)alkyl; P; Q is H, (C₁-C₁₄)alkyl or a suitable carboxy protecting group; n is 2 to about 20; I is 1-5, j is 0-4 and I+j is ≦5; or a pharmaceutically acceptable salt thereof. Specifically, i can be 1, j can be 0, M can be a positron emitter such as Fluorine-18, Bromine-76, Iodine-124 or a gamma emitter such as Iodine-123 or Iodine-131 and n can be about 6 to about 12.

The above discussion has demonstrated how the various variables associated with the cobalamin conjugates of the present invention can be independently varied to more particularly define specific classes of cobalamin conjugates encompassed by this invention. It is to be understood that the modification of one variable can be made independently of the modification of any other variable. Moreover, any number of embodiments can be defined by modifying two or more of the variables in such embodiments. A few of such embodiments are described below for purposes of exemplification.

Subembodiment 1:

X is 5′-deoxyadenosyl; M is a divalent heterocycle wherein the radical positions can be within the ring or a substituent to the ring such that at least one radical is on a heteroatom to form a dative bond with cobalt, optionally substituted by L-T; K is O, S, NJ¹, CR¹⁰⁰R¹⁰¹ or C(R¹⁰⁰)V⁸Z⁸; E is O or S; G¹ is hydrogen, alkyl, acyl, silyl, phosphate or L-T; Y¹, Y², Y³, Y⁴, Y⁵, Y and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z² , Z³, Z⁴, Z⁵, Z⁷ and Z′ independently are R¹⁰⁴, L-T or L-T′; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z¹, Z², Z³, Z⁴, Z⁵, Z⁷ and Z⁸, M or G¹ comprises a radionuclide; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 2:

X is 5′-deoxyadenosyl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z7 and Z8 independently are R¹⁰⁴ , L-T or L-T′; each L is independently a direct bond or the residue of a multivalent moiety that does not significantly impair the ability of the compound to bind transcobalamnin or intrinsic factor proteins; each T or T′ independently comprises the residue of one or more radionuclides; at least one of Z¹, Z², Z³, Z⁴, Z⁵, Z⁷ and Z⁸, M or G¹ comprises a radionuclide; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 3:

X is 5′-deoxyadenosyl; M is a divalent heterocycle wherein the radical positions can be within the ring or a substituent to the ring such that at least one radical is on a heteroatom to form a dative bond with cobalt, optionally substituted by L-T; K is O, S, NJ¹, CR¹⁰⁰R¹⁰¹ or C(R¹⁰⁰)V⁸Z⁸; E is O or S; G¹ is hydrogen, alkyl, acyl, silyl, phosphate or Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z², z or Z⁵ comprises a radionuclide, the remaining Z moieties retaining their natural vitamin B₁₂ configuration; J¹, J² and J₃ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 4:

X is hydrogen, cyano, amino, amido, hydroxyl, 5′-deoxyadenosyl, L-T, alkyl, alkenyl, alkynyl, cycloalkyl, aryl, aralkyl, heterocycle or heteroaryl or alkylheteroaryl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ′; CR¹⁰² R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z¹, Z², Z³, Z⁴, Z⁵, Z⁶, Z⁷, Z⁸, M and G¹ comprises a radionuclide; J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴and R¹⁵ retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 5:

X is hydrogen, cyano, amino, amido, hydroxyl, 5′-deoxyadenosyl, L-T, alkyl, alkenyl, alkynyl, cycloalkyl, aryl, aralkyl, heterocycle or heteroaryl or alkylheteroaryl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z², Z⁴ or Z⁵ comprises a radionuclide, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰² , R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 6:

X is hydrogen, cyano, amino, amido, hydroxyl, 5′-deoxyadenosyl, L-T, alkyl, alkenyl, alkynyl, cycloalkyl, aryl, aralkyl, heterocycle or heteroaryl or alkylheteroaryl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V independently are O, S or NJ′; CR¹² R¹⁰³ or a direct bond; Z¹, Z², Z³, Z⁴, Z⁵ , Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z², z⁴ or Z⁵ comprises a radionuclide, the remaining Z moieties retaining their natural vitamin B₁₂ configuration; JI, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰² , R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 7:

X is 5′-deoxyadenosyl; M, K, E and G¹ retain their natural vitamin B₁₂ configuration; Y¹, Y², Y³, Y⁴, Y⁵, Y⁶ and Y⁷ independently are O, S or NJ²; V¹, V^{2, V}³, V⁴, V⁵, V⁶, V⁷ and V⁸ independently are O, S or NJ²; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z¹, Z², Z³, Z⁴, Z⁵, Z⁷, Z⁸, M and G¹ comprises a radionuclide; J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹² , R¹³, R¹⁴ and R¹⁵ retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 8:

X is 5′-deoxyadenosyl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ3; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z², Z⁴ or Z⁵ comprises a radionuclide, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 9:

X is hydrogen, cyano, amino, amido, hydroxyl, 5′-deoxyadenosyl, L-T, alkyl, alkenyl, alkynyl, cycloalkyl, aryl, aralkyl, heterocycle or heteroaryl or alkylheteroaryl; M, K, E and G¹ retain their natural vitamin B₁₂ configuration; Y¹, Y², Y³, Y⁴, Y⁵, Y⁶ and Y⁷ independently are O, S or NJ′; V¹, V², V³, V⁴, V⁵, V⁶, V⁷ and V⁸ independently are O, S or NJ3; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z², Z⁴ or Z⁵ comprises a radionuclide, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ all retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 10:

X is 5′-deoxyadenosyl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰² R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z², Z⁴ or Z⁵ comprises a radionuclide, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴and R¹⁵ all retain their natural vitamin B₁₂ configuration; and R¹⁰⁰, R^{101, R}¹⁰² , R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiments 11-20:

Any one of subembodiments 1-10, wherein the linker has a substantially constant molecular weight.

Subembodiments 21-30:

Any one of subembodiments 1-10, wherein the linker is a polyamine of the following chemical structure: NR′(alkylene-NR′)ₙalkyleneNR′, wherein n is from 1 to 20, the carbon length of alkylene can vary within the n units and each R′ is independently hydrogen, lower alkyl or T.

Subembodiments 31-40:

Any one of subembodiments 1-10, wherein the linker is spermine, spennidine, decamethylene tetraamine or pentamethylene hexamine.

VI. Detectable Radionuclides

As used herein, a “detectable radionuclide” is any suitable radionuclide (i.e. radioisotope) capable of being detected in a diagnostic procedure in vivo or in vitro. Suitable detectable radionuclides include metallic radionuclides (i.e. metallic radioisotopes) and non-metallic radionuclides (i.e. non-metallic radioisotopes).

Suitable metallic radionuclides (i.e. metallic radioisotopes or metallic paramagnetic ions) include Antimony-124, Antimony-125, Arsenic-74, Barium-103, Barium-140, Beryllium-7, Bismuth-206, Bismuth-207, Cadmium-109, Cadmium-115m, Calcium-45, Cerium-139, Cerium-141, Cerium-144, Cesium-137, Chromium-51, Cobalt-55, Cobalt-56, Cobalt-57, Cobalt-58, Cobalt-60, Cobalt-64, Copper-67, Erbium-169, Europium-152, Gallium-64, Gallium-68, Gadolinium-153, Gadolinium-157 Gold-195, Gold-199, Hafnium-175, Haffium-175-181, Holmium-166, Indium-110, Indium-11, Iridium-192, Iron-55, Iron-59, Krypton-85, Lead-210, Lutetium-177, Manganese-54, Mercury-197, Mercury-203, Molybdenum-99, Neodymium-147, Neptunium-237, Nickel-63, Niobium-95, Osmium-185 +191, Palladium-103, Platinum-195m, Praseodymium-143, Promethium-147, Protactinium-233, Radium-226, Rhenium-186, Rhenium-188, Rubidium-86, Ruthenium-103, Ruthenium-106, Scandium-44, Scandium-46, Selenium-75, Silver-ilOm, Silver-111, Sodium-22, Strontium-85, Strontium-89, Strontium-90, Sulfur-35, Tantalum-182, Technetium-99m, Tellurium-125, Tellurium—132, Thallium-204, Thorium-228, Thorium-232, Thallium-170, Tin-113, Tin-114, Tin-117m, Titanium-44, Tungsten-185, Vanadium-48, Vanadium-49, Ytterbium-169, Yttrium-86, Yttrium-88, Yttrium-90, Yttrium-91, Zinc-65 and Zirconium-95.

The compounds of the invention can also comprise one or more (e.g. 1, 2, 3 or 4) non-metallic radionuclide which can be directly linked to a residue of the compound of formula I at any synthetically feasible site or can be linked to a residue of the compound of formula I, by a linker, at any synthetically feasible site. Suitable linkers are described herein. In addition, suitable points of attachment of a the compound of formula I for the non-metallic radionuclide, either directly or by a linker, are also described herein. The invention also provides compounds having more than one non-metallic radionuclide attached to a compound of formula I, either directly or by a linker.

Specifically, the non-metallic radionuclide can be a non-metallic paramagnetic atom (e.g. Fluorine-i 9); or non-metallic positron emitting radionuclide (e.g. Carbon-11, Fluorine-18, Iodine-12 or Bromine-76) or a nonmetallic gamma emitting radionuclide such as Iodine-123 or Iodine-131. Fluorine-19 is a suitable non-metallic paramagnetic for use the compounds of the present invention in part because there is typically little or no background noise associated with the diagnostic use of fluorine in the body of a mammal (e.g. human).

VII. Chelating Group

Chelating groups can be used to link radionuclides to the cobalamin conjugate of the present invention. Any suitable chelating group can be employed. Suitable chelating groups include those disclosed in U.S. Pat. No. 5,739,313. Other suitable chelating groups are the thiazoline derivatives disclosed in U.S. Pat. No. 6,083,966, the pyridinones disclosed in U.S. Pat. No. 5,892,029 and the catecholaurates disclosed in U.S. Pat. No. 5,514,695.

As used herein, a “therapeutic chelating group” is a chelating group comprising a metallic radionuclide (e.g. a metallic radioisotope) that possesses therapeutic efficacy against cancer or other neoplastic cells in vivo or in vitro. Any suitable chelating group can be employed.

Specifically, the therapeutic chelating group can be any of the carbonyl complexes disclosed in Waibel et al., Nature Biotechnology, 897-901, Vol. 17, September 1999; or Sattelberger et al., Nature Biotechnology, 849-850, Vol. 17, September 1999, further comprising a metallic radionuclide. More specifically, the therapeutic chelating group can be any of the carbonyl complexes disclosed in Waibel et al., Nature Biotechnology, 897-901, Vol. 17, September 1999; or Sattelberger et al., Nature Biotechnology, 849-850, Vol. 17, September 1999, further comprising Rhenium-186 or Rhenium-188.

In one embodiment, the chelating group can be NTA, HEDTA, DCTA, RP414, MDP, DOTATOC, CDTA, HYNIC, EDTA, DTPA, TETA, DOTA, DOTMP, DCTA, 15N4, 9N3, 12N3 or MAG3 (or another suitable polyamino acid chelator), which are described herein below or a phosphonate chelator (e.g. EDMT). In a preferred embodiment, the chelating group is DTPA.

DTPA is diethylenetriaminepentaacetic acid; TETA is 1,4,8,11-tetraaza-cyclo-tetradecane-N,N′,N″,N′″-tetraacetic acid; DOTA is 1,4,7,10-tetraaza-cyclododecane-N,N,N″,N′″-tetraacetic acid; 15N4 is 1,4,8,12-tetraazacyclo-pentadecane-N,N′,N″,N′″-tetra-acetic acid; 9N3 is 1,4,7-triazacyclononane-N,N′,N″-triacetic acid; 12N3 is 1,5,9-triazacyclo-dodecane-N,N′,N″-triacetic acid; polyaminoacid chelators, such as MAG3 is (N-(N-(N-((benzoylthio)acetyl)glycyl)glycyl)glycine); and DCTA is a cyclohexane-based metal chelator of the formula

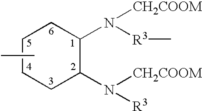

wherein R³ may by (C₁-C₄)alkyl or CH₂CO₂—, which may be attached through positions 4 or 5 or through the group R³ and which carries from 1 to 4 detectable metal or nonmetal cations (M), monovalent cations or the alkaline earth metals. Thus, with metals of oxidation state +1, each individual cyclohexane-based molecule may carry up to 4 metal cations (where both R³ groups are CH₂COOM). As is more likely, with higher oxidation states, the number of metals will decrease to 2 or even 1 per cyclohexane skeleton. This formula is not intended to limit the molecule to any specific stereochemistry.

NTA, HEDTA and DCTA are disclosed in Poster Sessions, Proceedings of the 46th Annual Meeting, J. Nuc. Med., p. 316, No. 1386. RP414 is disclosed in Scientific Papers, Proceedings of the 46th Annual Meeting, J. Nuc. Med., p. 123, No. 499. MDP is disclosed in Scientific Papers, Proceedings of the 46th Annual Meeting, J. Nuc. Med., p. 102, No. 413. DOTATOC is disclosed in Scientific Papers, Proceedings of the 46th Annual Meeting, J. Nuc. Med., p. 102, No. 414 and Scientific Papers, Proceedings of the 46th Annual Meeting, J. Nuc. Med., p. 103, No. 415. CDTA is disclosed in Poster Sessions, Proceedings of the 46th Annual Meeting, J. Nuc. Med., p. 318, No. 1396. HYNIC is disclosed in Poster Sessions, Proceedings of the 46th Annual Meeting, J. Nuc. Med., p. 319, No. 1398.

Bifunctional chelators (i.e. chelating groups) based on macrocyclic ligands in which conjugation is via an activated arm attached to the carbon backbone of the ligand can also be employed as a chelating group, as described by M. Moi et al., J. Amer. Chem., Soc., 49, 2639 (1989) (2-p-nitrobenzyl-1,4,7,10-tetraazacyclododecane-N,N′,N″,N′″-tetraacetic acid); S. V. Deshpande et al., J. Nuc. Med., 31, 473 (1990); G. Kuser et al., Bioconj. Chem., 1, 345 (1990); C. J. Broan et al., J. C. S. Chem. Comm., 23, 1739 (1990); and C. J. Anderson et al., J. Nuc. Med. 36, 850 (1995) (6-bromoacetamido-benzyl-1,4,8,11-tetraazacyclotetadecane-N,N′,N″,N′″-tetraacetic acid (BAT)).

In addition, the chelator or chelating group can be any of the chelating groups disclosed in Scientific Papers, Proceedings of the 46th Annual Meeting, J. Nuc. Med., Wednesday, Jun. 9, 1999, p. 124, No. 500.

Specifically, the chelating group can be any one of the carbonyl complexes disclosed in Waibel et al., Nature Biotechnology, 897-901, Vol. 17, September 1999; or Sattelberger et al., Nature Biotechnology, 849-850, Vol. 17, September 1999.

Specifically, the detectable chelating group can be any one of the carbonyl complexes disclosed in Waibel et al., Nature Biotechnology, 897-901, Vol. 17, September 1999; or Sattelberger et al., Nature Biotechnology, 849-850, Vol. 17, September 1999, further comprising a metallic radionuclide. More specifically, the detectable chelating group can be any one of the carbonyl complexes disclosed in Waibel et al., Nature Biotechnology, 897-901, Vol. 17, September 1999; or Saftelberger et al., Nature Biotechnology, 849-850, Vol. 17, September 1999, further comprising Technetium-99m, Rhenium-186 or Rhenium-188.

VIII. Cardiovascular Agent

As used herein, a “cardiovascular agent” is any compound useful to treat one or more abnormal conditions associated with the cardiovascular system. Suitable cardiovascular agents are disclosed, e.g. in Physician's Desk Reference (PDR), Medical Economics Company (Montvale, N.J.), (53rd Ed.), 1999; Maya Medical Center Formulary., Unabridged Version, Mayo Clinic (Rochester, Minn.), January 1998; Yale University School of Medicine Heart Book: Chapter 23 Cardiovascular Drugs, http://www.info.med.yale.edu/library/heartbk, Apr. 16, 1999; Merck Index, An Encyclopedia of Chemicals, Drugs and Biologicals, (11th Ed.), Merck & Co., Inc. (Rahway, N.J.), 1989; and references cited therein.

Suitable cardiovascular agents include blood modifiers, adrenergic blockers (peripheral), adrenergic stimulants (central), alpha/beta adrenergic blockers, angiotensin converting enzyme (ACE) inhibitors, angiotensin II receptor antagonists, anti-arrhythmics (groups I, II, I and IV), miscellaneous anti-arrhythmics, 30 anti-lipemic agents, beta adrenergic blocking agents, calcium channel blockers, diuretics, hypertensive emergency agents, inotropic agents, miscellaneous cardiovascular agents, rauwolfia derivatives, vasodilators and vasopressors.

It is appreciated that those skilled in the art understand that the cardiovascular agent useful in the present invention is the biologically active compound present in any of the cardiovascular compositions disclosed above. For example, Cardizem (diltiazem HCl) is typically available as an injectable, as a sustained release capsule and as a direct compression tablet. The cardiovascular agent, however, is (+)-cis-1,5-benzothiazepin-4(5H)one-3-(acetyl-oxy)-5-[2-(dimethyl-amino)ethyl]-2,3-dihydro-2-(4-methoxyphenyl)-monohydrochloride. Physician's Desk Reference (PDR), Medical Economics Company (Montvale, N.J.), (53rd Ed.), pp. 1311-1318, 1999.

As used herein, a “residue of cardiovascular agent” is a radical of a cardiovascular agent having one or more open valences. Any synthetically feasible atom or atoms of the cardiovascular agent can be removed to provide the open valence, provided bioactivity is substantially retained when the radical is attached to a residue of a compound of formula (I). Based on the linkage that is desired, one skilled in the art can select suitably fluctionalized starting materials that can be derived from a cardiovascular agent using procedures that are known in the art.

Any of the cardiovascular agents listed in the Background, any listed below or any other such agent known or discovered to exhibit a cardiovascular effect that can be more effectively delivered by conjugation to a TC- or IF-binding agent can be used in accordance with this invention.

In an alternative embodiment, any of the cardiovascular agents listed in the Background, listed below or any other such known agents can be used in combination with a TC- or IF-binding agent/cardiovascular agent to achieve a combination therapeutic effect.

The cardiovascular agent can be bound through a covalent bond, a dative bond, a coordination bond, complexation (such as found in a bound antibody/epitope) or ionic bond. Covalent bonding is preferred over ionic bonding, however, a tightly held ionic bond may be suitable. Below are nonlimiting examples of how agents can be attached to carriers. Other routine means are known to those skilled in the art and are assumed included within the scope of the invention.

Antibody

In general, an antibody can be linked preferably in its Fc or constant, region in a manner that does not effect the binding of the antibody to a moiety. Thus, the antibody can be bound through a functional group, preferably an amide of the antibody, to the TC- or IF-binding agent, using standard chemical reactions for covalent bond formation.

ReoPro (abciximab)—FAB fragment of chimeric human-murine monoclonal antibody 7E3

Neupogen (filgrastim; GCSF)—recombinant human Gamma-CSF

Leukine (sagramostim; GM-CSF; CSF-2; Prokine; Imunex)—recombinant human GM-CSF

Trinsicon—hematinic concentrate with intrinsic factor

Erythropoietin (Epogen, epoetin alfa; Eprex; EPO; R-HuEPO; Procrit; ESF; Ep; Erypo; Marogen; Epoade; Espo)—recombinant human erthropoeitin

LoCholest (cholestyramine; Questran; Cholybar; dowex l-x2-cl; MK-135; cuemid; quantalan)

Free Amine or Amide

The following are examples of cardiovascular agents that contain an amine or an amide group and thus can be linked to the TC- or IF-binding agent through that functional moiety, using standard chemical reactions for covalent bond formation to a nitrogen atom.

Agrylin (anagrelide hydrochloride);

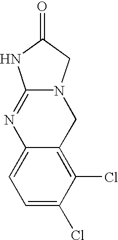

Nascobal (cyanocobalamin, vitamin B₁₂);

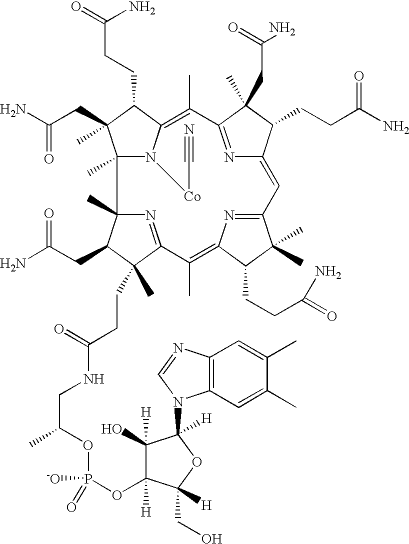

Heparin sodium (Dalteparin Sodium; Ardeparin sodium; Enoxaparin sodium; Fragmin—group of straight-chain anionic mucopolysaccharides, i.e. a glycoaminoglycan)

Cardura (doxazosin mesylate);

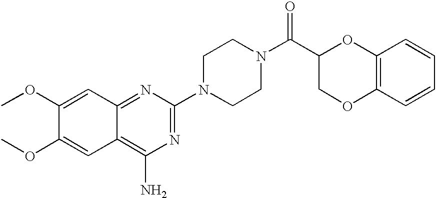

Hytrin (terazosin hydrochloride);

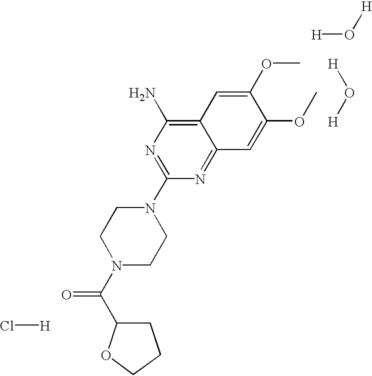

Lovenox (enoxaparin sodium—2-O-sulfo-4-enepyranosuronic acid group and a 2-N,6-30-O-disulfo-D-glucosamine)

Normiflo (ardeparin sodium)—polymer chains of D-glucosamine derivatives and hexuronic acid

Minipress (prazosin hydrochloride; Pressin)

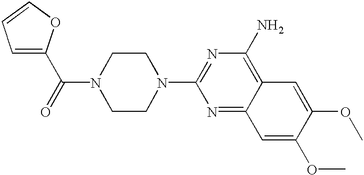

Polythiazide (Renese; drenusil; nehpril; P2525)—with prozosin then, Minizide

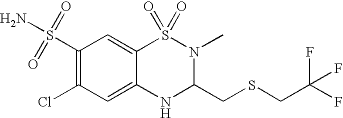

Aldoclor (methyldopa and chlorothiazide sodium=Diuril);—see above and

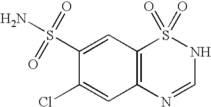

hydrochlorothiazide (HCT™; HydroDURIL; HCTZ; Microzide)—with methyldopa, then Aldoril

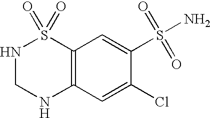

Catapres-TTS (clonidine; Catapres; Combipres; Catapre-TTS; Catapres-tts)

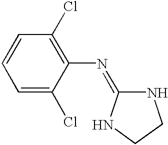

Clorpres or Combipres (clonidine hydrochloride and chlorthalidone=Hygroton; Thalitone); see above and

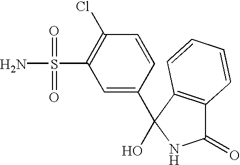

Tenex (guanfacine)

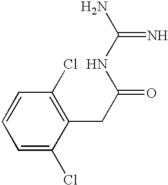

Hylorel (guanadrel sulfate);

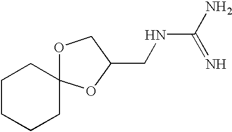

Coreg (carvedilol);

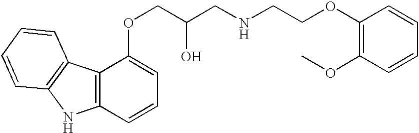

Labetalol (Normodyne; Trandate; Presolol)

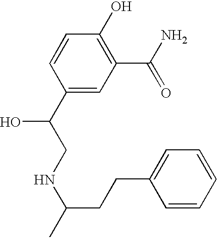

Ethmozine (moricizine hydrochloride);

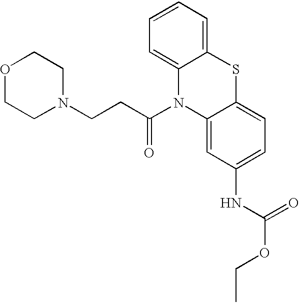

Mexitil (mexiletine hydrochloride; Katen; Ritahnex);

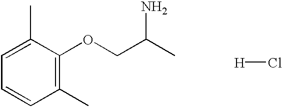

Norpace (disopyramide phosphate); and its related controlled release formulation Norpace CR

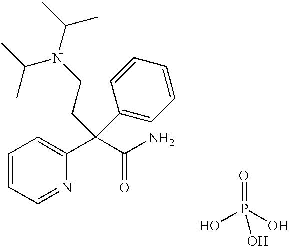

Procanbid (procainamide hydrochloride extended-release tablets);

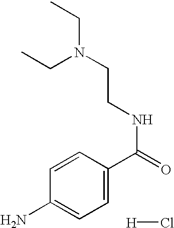

Tambocor (flecainide acetate);

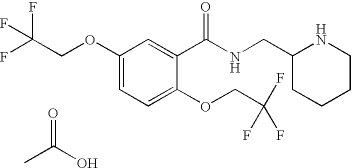

Brevibloc (esmolol hydrochloride; Esmolol; Ethiofos; Amifostine);

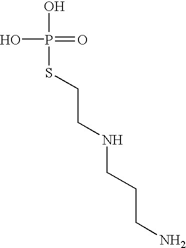

Colestid (microionized colestipol hydrochloride);

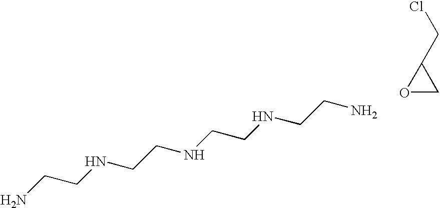

Adalat (nifedipine; Procardia; citilat; oxcord; bay 1040; cordipin; Nifecard; Nyefax); and extended release formulation Procardia XL

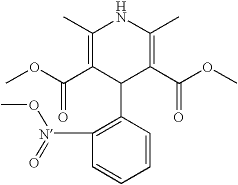

Cardene (nicardipine hydrochloride; Perdipene);

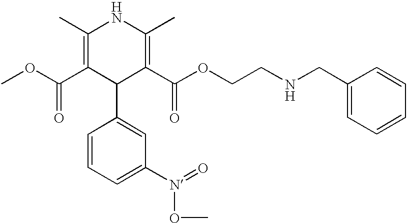

DynaCirc (isradipine);

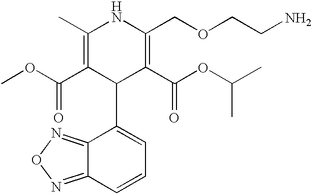

Nimotop (nimodipine; Admon; Periplum);

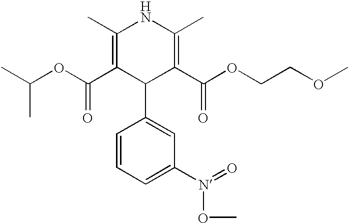

Norvasc (amlodipine besylate)

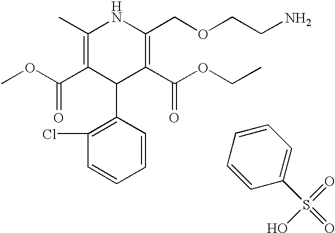

Plendil (felodipine);

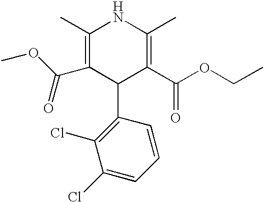

Sular (nisoldipine);

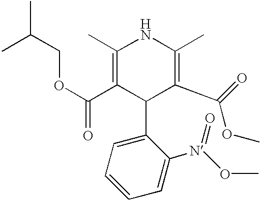

Daranide (dichlorphenarnide);

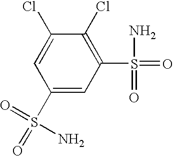

Demadex (torsemide);

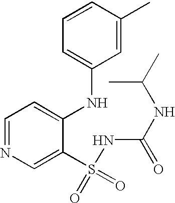

Dyrenium (triamterene; Dyazide; ademine; Diren; Dytac; jatropur; noridil; pterofen; pterophene; skf 8542; taturil; teriam; teridin; triampur; triamteril; tri-span; triteren; urocaudal; noridyl);

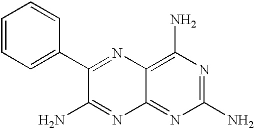

Midamor (amiloride; Moduretic; amipramizide; amiprazide; nilurid; amipramidine; guanamprazine hydrochloride; colectril; modamide; arumil);

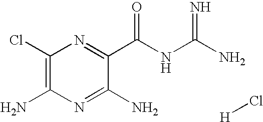

Diucardin (hydroflumethazide; Saluron);

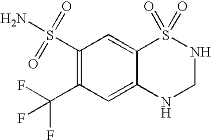

Enduron (methyclothiazide; Aquatensen);

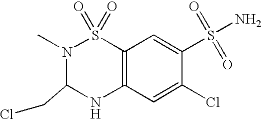

Mykrox (metolazone; Diulo; Zaroxolyn);

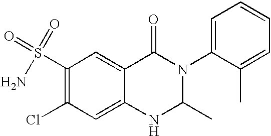

Primacor (milrinone lactate injection)

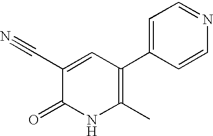

Inversine (Mecamylamine HCl; Versamine);

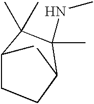

Diupres (chlorothiazide and reserpine); or Hydropres (hydrochlorothiazide and reserpine).

[chemical structure]

ProAmatine (midodrine hydrochloride);

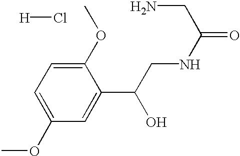

Free Hydroxyl

The following are examples of cardiovascular agents that contain an alcohol moiety and thus can be linked to the TC- or IF-binding agent through that functional moiety, using standard chemical reactions for covalent bond formation by derivatization of a hydroxyl.

Coumadin (Warfarin, Compound 42; Panwarfin; Sofarin; Rodex; zoocoumarin; Co-Rax; Cov-R-Tox; Kypfarin; Liqua-Tox; RAX; Tox-Hid; athrombin-k; brumolin; dethnel; fasco fascrat; kumader; kumadu; mar-frin; martin's mar-frin; maveran; ; prothromadin; Rosex; solfarin; twin ligh; vampirinip; Benzopyran-2-one, ; Marevan)

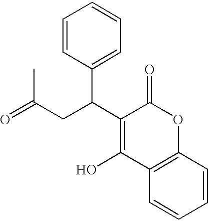

Persantine (dipyridamole; Dipridacot)

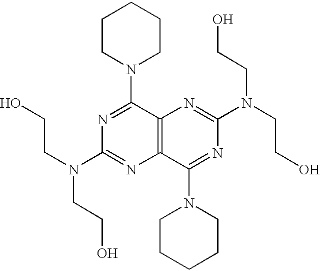

Anadrol-50 (oxymetholone; plenastril; protanabol; roboral; synasteron; zenalosyn)

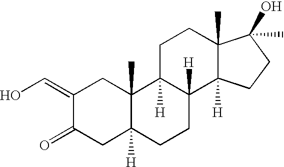

Cozaar (losartan potassium)—2-butyl-4-chloro-1-[p-(o-1H-tetrazol-5-ylphenyl)benzyl]-imidazole-5-methanol monopotassium salt.

Cardioquin (quinidine polygalacturonate; Quinidine Conchinine; Quinicardine; Quinidex; Quinaglute; pitayine; Kinidin)

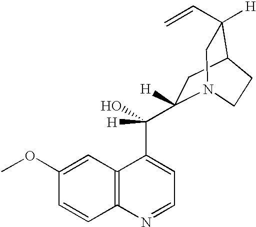

Quinidex (quinidine sulfate); Quinitex; Quinora

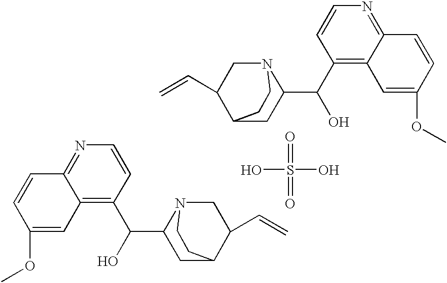

Rythmol (propafenone hydrochloride);

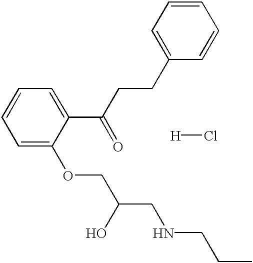

Betapace (sotalol HCl)

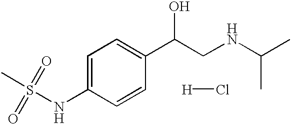

Inderal (Popranolol HCl; Deralin);

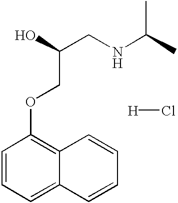

Sectral (acebutolol; Monitan)

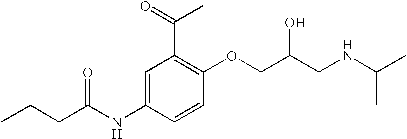

Adenocard (adenosine)

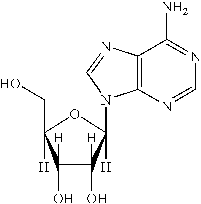

Lanoxicaps (digoxin; Lanoxin);

[chemical structure]

Mevacor (lovastatin);

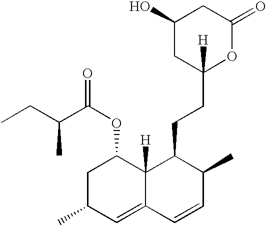

Zocor (simvastatin; Lipex);

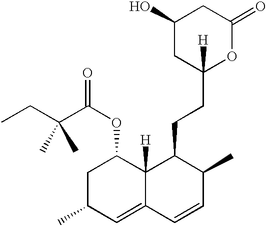

Blocadren (Timolol; Tenopt; Timoptol; Timpilo)

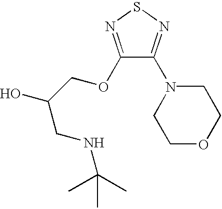

Cartrol (carteolol hydrochloride);

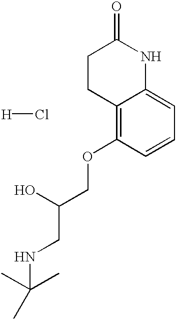

Inderal (propranolol hydrochloride; Deralin);

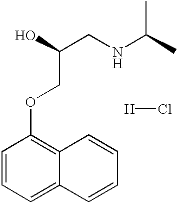

Kerlone (betaxolol hydrochloride; Betoptic);

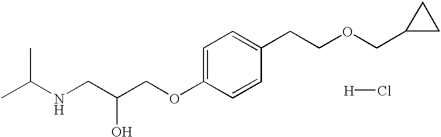

Levatol (Penbutolol sulfate);

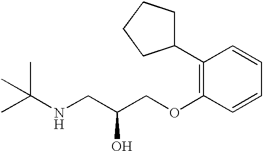

Lopressor (metropolol tartrate); Toprol-XL (metoprolol succinate, extended release)

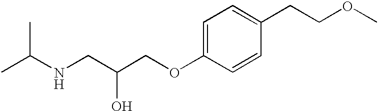

Sectral (acetebutolol hydrochloride; Monitan; Sectral);

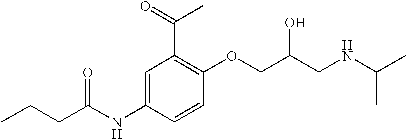

Tenormin (atenolol; Anselol; Noten; Tenlol);

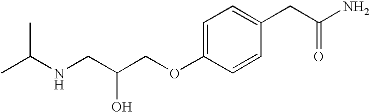

Zebeta (bisoprolol fumurate; Ziae)

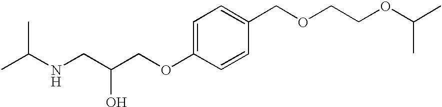

Dobutrex (dobutamine hydrochloride);

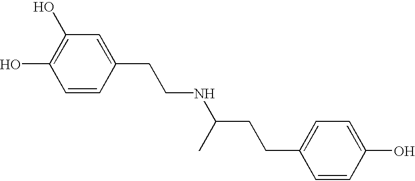

Monoket (isosorbide mononitrate)

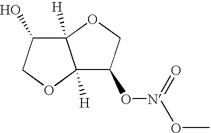

Corlopam (fenoldopam mesylate);

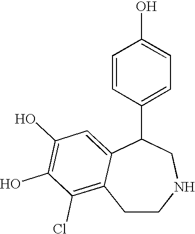

Ana-Kit (epinephrine; EpiPen; adrenalin)

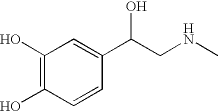

Free Carboxylic Acid

The following are examples of cardiovascular agents that contain a carboxylic acid moiety and thus can be linked to the TC- or IF-binding agent through that functional moiety, using standard chemical reactions for covalent bond formation by derivatization of a carboxylic acid.

Integrilin (eptifibatide)—N6-(arninoiminomethyl)-N2-(3-mercapto-1-oxopropyl-L-lysyl-glycyl-L-trptophyl-L-prolyl-L-cysteinamaide, intramolecularly cyclized via a disulfide bond.

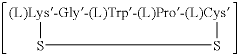

Aggrastat (tirofiban hydrochloride monohydrate)—N-(butylsulfonyl)-O-[4-(4-piperidinyl)butyl]-L-tyrosine monohydrochloride monohydrate

Ecotrin (enteric-coated aspirin; Acetylsalicylic acid) Halfprin (enteric-coated aspirin);

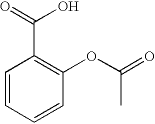

Flolan (epoprostenol sodium; Prostaglandin 12, Prostacyclin; PGI2);

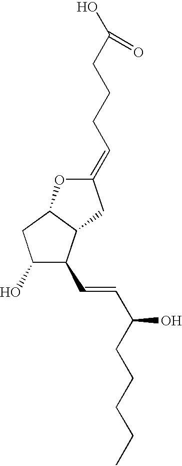

Aldomet (methyldopa); and its related Aldomet ester HCl (methyldopate HCl);

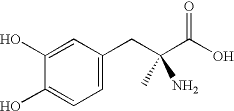

Accupril (quinapril hydrochloride, Asig)

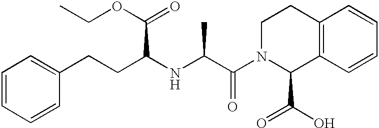

Altace (ramipril);

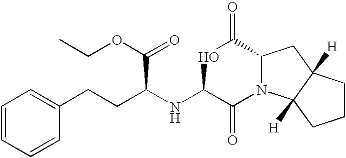

Captopril;

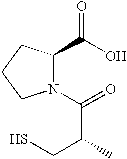

Lotensin (benazepril hydrochloride);

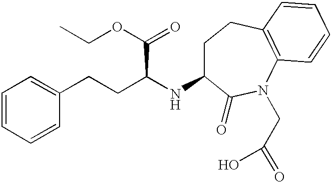

Mavik (trandolapril; Gopten; Odrik)—(2S,3aR,7aS)-1[(S)-N-[(S)-1-carboxy-3-phenylpropyl]-alanyl]hexahydro-2-indolinecarboxylic acid 1-ethyl ester

Monopril (fosinopril sodium tablets);

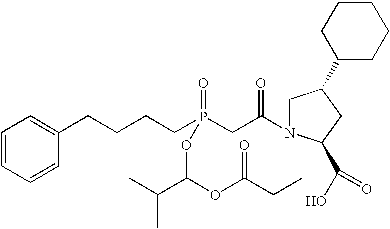

Prinivil (Lisinopril)—(S)-1-[N²-(1-carboxy-3-phenylpropyl-L-lysyl]-L-proline dehydrate.

Univasc (moexipril hydrochloride)—[3S-[2[R*(R*)], 3R*]]-2-[2-[[1-(ethoxycarbonyl)-3-phenylpropyl] amino]-1-oxopropyl]-1,2,3,⁴-tetrahydro-6,7-dimethoxy-3-isoquinolinecarboxylic acid, monohydrochloride.

Vasotec (enalapril maleate);

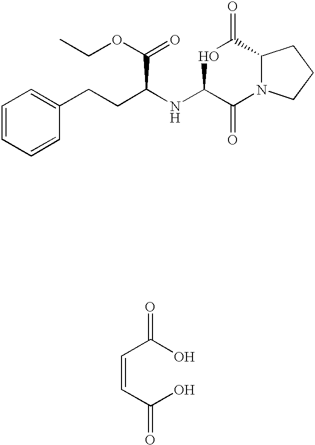

Zestril (lisinopril; Prinivil)

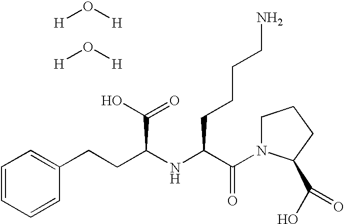

Atacand (candesartan cilexetil)—(±)-1-[[(cyclohexyloxy)carbonyl]oxy]ethyl 2-ethoxy-1-[[2′-(1H-tetrazol-5-yl) [1,1′-biphenyl]-4-yl]methyl]-1H-benzimidazole-7-carboxylate.

Diovan (Valsartan)—N-(1-oxopentyl)-N-[[2′-(1H-tetrazol-5-yl) [1,1′-biphenyl]-4-yl]methyl]-L-valine.

Corvert (ibutilide fiunarate injection)—Methane-sulfonamide, N-{4-{4-(ethyl-heptylamino)-1-hydroxy butyl}phenyl}, (+), (−), (E)-2-butenedioate (1:0.5) (hemifuimarate salt)

Lopid (gemfibrozil; Jezil);

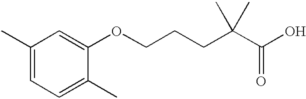

Baycol (cerivastatin sodium tablets)—sodium [S-[R*, S*-(E)]]-7-[4-(4-fluorophenyl)-5-methoxymethyl)-2,6 bis(I-methylethyl)-3-pyridinyl]-3,5-dihydroxy-6-heptenoate

Lescol (fluvastatin sodium; vastin);

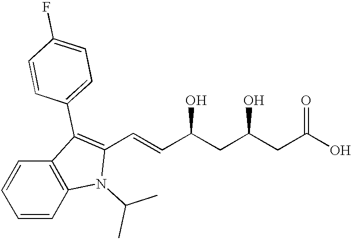

Lipitor (atorvastatin calcium; [R-(R*, R*)]-2-(4-fluorophenyl)-D3,8-dihydroxy-5-(1-methylethyl)-3-phenyl-4-[(phenylamino)carbonyl]-1H1-pyrrole-l1-heptanoic acid, calcium salt (2: 1) trihydrate);

Pravachol (pravastatin sodium);

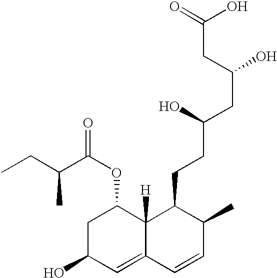

Niaspan (nicotinic acid; Niacin; Nia-bid; NIAC; Niacels; Niacor; Nicobid; Nicolar)

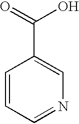

Edecrin (ethacrynic acid; Dethacrynic acid; mingit; MK-595; otacril; redmax; taladren; uregit; crinuryl; hydromedin);

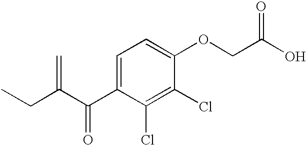

Lasix (furosemide; Myrosemide; furosedon; lasilix; aisemide; aluzine; beronald; desdemin; diural; dryptal; errolon; eutensin; frusid; fulsix; fulvamide; furanthril; furanthryl; furantril; furesis; Fusid; hydro-rapid; katlex; lowpstron; macasirool; profemin; radonna; rosemide; Salix; seguril; transit; trofurit; urosemide; LB 502);

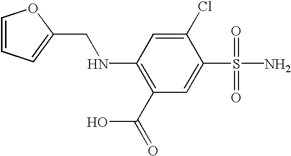

Hyperstat (diazoxide; Proglycem)

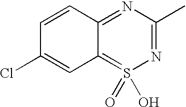

Demser (metyrosine)—(−)-α-methyl-L-tyrosine

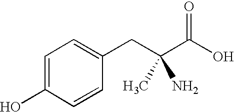

Regitine (phentolamine mesylate)—4,5-dihydro-2-[N(m-hydroxy-phenyl)-N-(p-methyl-phenyl)aminomethyl]-1H-imidazole 1:1 methane sulfonate.

Aramine (Metaraminol bitartrate)—[R-(R*, S*)]-α-(1-aminoethyl)-3-hydroxybenzene methanol [R(R*, R*)]-2,3-dihydroxy butanedioate (1:1)

Miscellaneous

The following cardiovascular agents do not have readily available functional groups to derivatize for covalent attachment to the TC- or IF-binding agent or linker, but can be attached through a suitable ionic bond with close salt formation, wherein the carrier or linker contains an appropriate counterion.

Plavix (clopidogrel bisulfate);

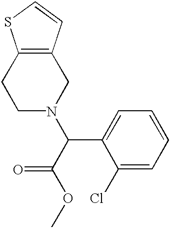

Dibenzyline (phenoxybenzamine);

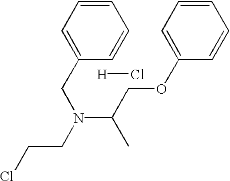

Ticlid (ticlopidine hydrochloride; Ticlodone)

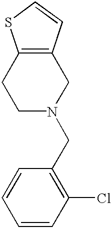

Avapro (irbesartan)-2-butyl-3-[[2′-(1H-tetrazol-5-yl) [1,1 ′-biphenyl]-4-yl]methyl]-1,3-diazaspiro [4,4] non-1-en-4-one.

Tonocard (tocainide HCL; nicethamid; nicetamide; anacardone; astrocar; betapyrimidum; camphozone; cardamine; cardiamid; cardiamine; citocor; coracon; coraethamidum; coramine; corazone; cordiamin; cordynil; corediol; coretone; cormid; comotone; corvitan; corvotone; danamine; diethyl-nicotamide)

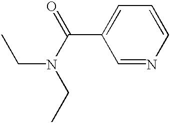

Cordarone (amiodarone; Aratac; Pacerone);

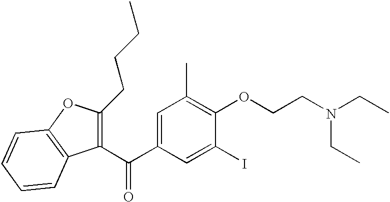

Calan (verapamil);

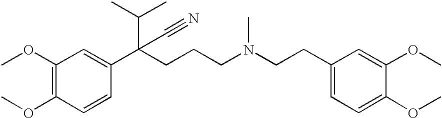

Cardizem (diltiazem HC1; Dilacor XR; Cardcal; Coras; Tiazac);

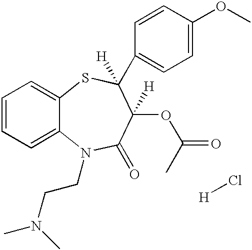

Atromid-S (clofibrate; Abitrate);

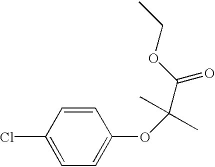

TriCor (fenofibrate capsules; Proctofene; Sedufen)

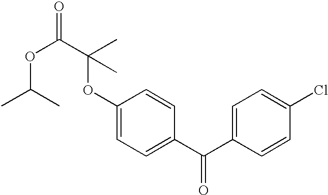

Calan (verapamil hydrochloride; Isoptin; Verelan; Covera HS; Tolmentin)

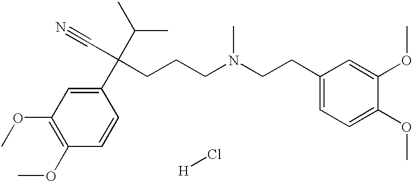

Vascor (bepridil hydrochloride; Bepadin);

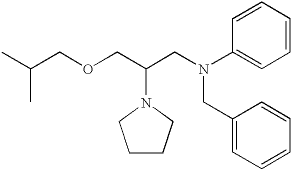

Aldactone (Spironolactone; Spiractin);

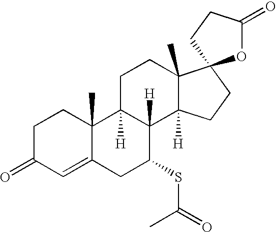

Nitroglycerin (Deponit; Nitro-Bid; Nitro-Dur; Nitrostat; Transderm-Nitro; Buccal; Nitrocap; Nitrocine; Nitrospan; NIONG; Nitronet; Nitrong parenteral; Nitroject; Nitrol; Tridil sublin; glonoin; trinitrin; S.N.G.; Adesitrin; Angibid; Angiolingual; Anginine; Angorin; Nitro-disc; Nitrogard; Minitran; Nitradisc; Nitrolingual [with Dichlorodifluoromethane and Dichlorotetrafluoromethane]);

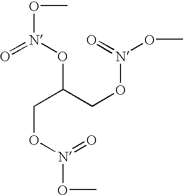

Isordil (isosorbide dinitrate; Sorbitrate; ISMO; Iso-bid; Isonate; Isordil; Isotrate; Indur; Isogen; Sorbidin); and sustained release formulation, Dilatrate-SR

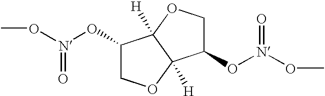

Vasoxyl (methoxamine hydrochloride)

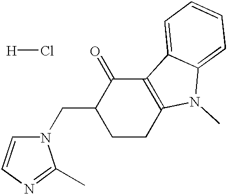

IX. Synthetic Techniques

Various synthetic techniques are known for preparing the compounds of the present invention. For example, compounds wherein the residue of an imaging agent is directly linked to the 6-position of a compound of formula I (i.e. in which X is L-T and L is a direct bond) can be prepared by reducing a corresponding Co (III) compound of formula I to form a nucleophilic Co (I) compound and treating this Co (I) compound with a residue of a imaging agent (or a derivative thereof) comprising a suitable leaving group, such as a halide. Similarly, compounds wherein X is L-T and L is other than a direct bond can be prepared by preparing a nucleophilic Co (I) species as described herein above and reacting it with a linker comprising a suitable leaving group, such as a halide. Peptides and amino acids can be attached to the 6-position by reducing a corresponding Co (III) compound of formula I to form a nucleophilic Co (I) compound and treating the Co (I) compound with a suitable alkylating agent comprising an amino acid or peptide.

Coupling of L-T to the ribose moiety at K or G¹ may be accomplished by activating the natural OH at either K or G¹ with a suitable reagent such as succinic anhydride, to yield a reactive group such as a carboxylate. This technique is described in detail in Toraya, Bioinorg. Chem. 4:245-255,1975.

Coupling of L-T to M can be accomplished using techniques described in detail in Jacobsen, Anal. Biochem. 113:164-171, 1981.

The residue of vitamin B₁₂ or its analog can be prepared by any suitable means known in the art. For example, a monocarboxylic acid or dicarboxylic acid of cobalamin can be prepared as disclosed in U.S. Pat. No. 5,739,313. These compounds can be prepared by the mild acid hydrolysis of cyanocobalamin, which has been shown to yield a mixture of mono-, a dicarboxylic acid and one tricarboxylic acid. These carboxylic acids are derived from the propionamide side chains designated b, d- and e-, as discussed hereinabove, which are more susceptible to hydrolysis than the amide groups on acetamide side chains a-, c- and g-. The b-, d- and e-monocarboxylic acids can be separated by column chromatography. L. Anton et at., J. Amer. Chem. Soc.,102, 2215 (1980). See, also, J B. Armitage et al., L Chem. Sot., 3349 (1953); K. Bernhauer, Biochem. Z., 344, 289 (1966); H. P. C. Hogenkamp et al., Biochemistry, 14, 3707 (1975); and L. Ellenbogen, in “Cobalamin,” Biochem. and Pathophysiol, B. Babior, ed., Wiley, N.Y. (1975) at chapter 5.

Additional compounds, intermediates and synthetic preparations thereof are disclosed, for example, in Hogenkamp, H. et al., Synthesis and Characterization of nido-Carborane-Cobalamin Conjugates, Nucl. Med. & Biol., 2000, 27, 89-92; Collins, D., et al., Tumor Imaging Via Indium 111-Labeled DTPA-Adenosylcobalamin, Mayo Clinic Proc., 1999, 74:687-691.

Compound of Formula I/Cardiovascular Agent Linkage

The invention provides a compound of formula I (FIG. 1) directly linked to one or more cardiovascular agents, wherein X is CN, OH, CH3, adenosyl or a cardiovascular agent; or a pharmaceutically acceptable salt thereof.

The residue of a cardiovascular agent can be linked to the residue of a compound of formula I through an amide (e.g. —N(R)C(═O)— or —C(═O)N(R)—), ester (e.g. —OC(═O)— or —C(═O)O—), ether (e.g. —O—), amino (e.g. —N(R)—), ketone (e.g. —C(═O)—), thioether (e.g. —S—), sulfinyl (e.g. —S(O)—), sulfonyl (e.g. —S(O)₂—) or a direct (e.g. C—C bond) linkage, wherein each R is independently H or (C₁-C₆)alkyl. Such a linkage can be formed from suitably functionalized starting materials using synthetic procedures that are known in the art. Based on the linkage that is desired, one skilled in the art can select suitably functional starting materials that can be derived from a residue of a compound of formula I and from a given residue of a cardiovascular agent using procedures that are known in the art.

The residue of the cardiovascular agent can be directly linked to any synthetically feasible position on the residue of a compound of formula I. Suitable points of attachment include, for example, the b-carboxamide, the d-carboxamide and the e-carboxamide (illustrated in FIG. 1), as well as the 6-position (the position occupied by X in FIG. 1) and the 5′-hydroxy and the 3′-hydroxy groups on the 5-membered sugar ring, although other points of attachment are possible. U.S. Pat. No. 5,739,313 discloses compounds (e.g. cyanocobalamin-b-(4-aminobutyl)amnide, methylcobalamin-b-(4-aminobutyl)amide and adenosylcobalamin-b-(4-aminobutyl)amide) that are useful intermediates for the preparation of compounds of the present invention.

Compounds wherein the residue of a cardiovascular agent is linked to the 6-position of a compound of formula I can be prepared by reducing a corresponding Co (m) compound of formula I to form a nucleophilic Co (I) compound and treating this Co (I) compound with a residue of a cardiovascular agent (or a derivative thereof) comprising a suitable leaving group, such as a halide (e.g. a chloride).

The invention also provides compounds having more than one residue of a cardiovascular agent or agents directly linked to a compound of formula I. For example, the residue of a cardiovascular agent can be directly linked to a residue of the b-carboxamide of the compound of formula I and a residue of another cardiovascular agent can be directly linked to a residue of the d- or e-carboxamide of the compound of formula I. In addition, the residue of a cardiovascular agent can be directly linked to the 6-position of the compound of formula I and a residue of another cardiovascular agent can be directly linked, for example, to a residue of the b-, d- or e-carboxamide of the compound of formula I.

Compound of Formula I/Linker/Cardiovascular Agent Linkage

In addition to being directly linked to the residue of a compound of formula I, the residue of a cardiovascular agent can also be linked to the residue of a compound of formula I by a suitable linker. The structure of the linker is not crucial, provided the resulting compound of the invention has an effective therapeutic index as a cardiovascular drug and preferably will localize in or near the cardiovascular system. Suitable linkers are disclosed, for example, in U.S. Pat. No. 5,735,313; U.S. application Ser. No. 60/129,733 filed Apr. 16, 1999; U.S. application Ser. No. 60/159,874 filed Oct. 15, 1999; U.S. application Ser. No. 60/159,753 filed Oct. 15, 1999; U.S. application Ser: No. 60/159,873 filed Oct. 15, 1999; and references cited therein.

Suitable linkers include linkers that separate the residue of a compound of formula I and the residue of a cardiovascular agent by about 5 angstroms to about 200 angstroms, inclusive, in length. Other suitable linkers include linkers that separate the residue of a compound of formula I and the residue of a cardiovascular agent by about 5 angstroms to about 100 angstroms, inclusive, in length, as well as linkers that separate the residue of a compound of formula I and the residue of a cardiovascular agent by about 5 angstroms to about 50 angstroms or by about 5 angstroms to about 25 angstroms, inclusive, in length.

The linker can be linked to any synthetically feasible position on the residue of a compound of formula I. Suitable points of attachment include, for example, a residue of the b-carboxamide, a residue of the d-carboxamide, a residue of the e-carboxamide, the 6-position (i.e. the position occupied by X in the compound of formula I), as well as a residue of the 5′-hydroxy group and a residue of the 3′-hydroxy group on the 5-membered sugar ring, although other points of attachment are possible. Based on the linkage that is desired, one skilled in the art can select suitably functionalized starting materials that can be derived from a compound of formula I and a cardiovascular agent using procedures that are known in the art.

The linker can conveniently be linked to the residue of a compound of formula I or to the residue of a cardiovascular agent through an amide (e.g. —N(R)C(═O)— or —C(═O)N(R)—), ester (e.g. —OC(═O)— or —C(═O)O—), ether (e.g. —O—), ketone (e.g. —C(═O)—) thioether (e.g. —S—), sulfmyl (e.g. —S(O)—), sulfonyl (e.g. —S(O)₂—), amino (e.g. —N(R)—) or a direct (e.g. C—C) linkage, wherein each R is independently H or (C₁-C₆)alkyl. The linkage can be formed from suitably functionalized starting materials using synthetic procedures that are known in the art. Based on the linkage that is desired, one skilled in the art can select suitably functional starting materials that can be derived from a residue of a compound of formula I, a residue of a cardiovascular agent and from a given linker using procedures that are known in the art.

Specifically, the linker can be a divalent radical of the formula W-A-Q wherein A is (C₁-C₂₄)alkyl, (C₂-C₂₄)alkenyl, (C₂-C₂₄)alkynyl, (C₃-C₈)cycloalkyl or (C₆-C₁₀)aryl, wherein W and Q are each independently —N(R)C(═O)—, —C(═O)N(R)—, —OC(═O)—, —C(═O)O—, —O—, —S—, —S(O)—, —S(O)₂—, —N(R)—, —C(═O)— or a direct bond (i.e. W and/or Q is absent); wherein each R is independently H or (C₁-C₆)alkyl.

Specifically, the linker can be a divalent radical of the formula W-(CH₂)ₙ-Q wherein, n is between about 1 and about 20, between about 1 and about 15, between about 2 and about 10, between about 2 and about 6 or between about 4 and about 6; wherein W and Q are each independently —N(R)C(═O)—, —C(—O)N(R)—, —OC(═O)—, —C(═O)O—, —O—, —S—, —S(O)—, —S(O)₂—, —C(═O)—, —N(R)— or a direct bond (i.e. W and/or Q is absent); wherein each R is independently H or (C1-C6)alkyl.

Specifically, W and Q can each independently be —N(R)C(═O)—, —C(═O)N(R)—, —OC(═O)—, —N(R)—, —C(═O)O—, —O— or a direct bond (i.e. W and/or Q is absent).

Specifically, the linker is a divalent radical, i.e. 1,ω-divalent radicals formed from a peptide or an amino acid. The peptide can comprise 2 to about 25 amino acids, 2 to about 15 amino acids or 2 to about 12 amino acids.

Specifically, the peptide can be poly-L-lysine (i.e. [—NHCH[(CH₂)₄NH₂]CO—]ₘ—Q, wherein —Q is H, (C₁-C₁₄)alkyl or a suitable carboxy protecting group; and wherein m is about 2 to about 25. Specifically, the poly-L-lysine can contain about 5 to about 15 residues (i.e. m is between about 5 and about 15). More specifically, the poly-L-lysine can contain about 8 to about 11 residues (i.e. m is between about 8 and about 11).

Specifically, the peptide can be poly-L-glutamic acid, poly-L-aspartic acid, poly-L-histidine, poly-L-ornithine, poly-L-serine, poly-L-threonine, poly-L-tyrosine, poly-L-leucine, poly-L-lysine-L-phenylalanine or poly-L-lysine-L-tyrosine.

Specifically, the linker can be prepared from 1,6-diaminohexane H₂N(CH₂)₆NH₂, 1,5-diaminopentane H₂N(CH₂)₅NH₂, 1,4-diaminobutane H₂N(CH₂)₄NH₂ or 1,3-diaminopropane H₂N(CH₂)₃NH₂.

Compounds wherein the linker is linked to the 6-position of a compound of formula I can be prepared by preparing a nucleophilic Co (I) species as described herein above and reacting it with a linker comprising a suitable leaving group, such as a halide (e.g. a chloride).

The invention also provides compounds having more than one cardiovascular agent attached to a compound of formula I, each through a linker. For example, the residue of a cardiovascular agent can conveniently be linked, through a linker, to a residue of the b-carboxamide of the compound of formula I and a residue of another cardiovascular agent can conveniently be linked, through a linker, to a residue of the d- or e-carboxamide of the compound of formula I. In addition, the residue of a cardiovascular agent can conveniently be linked, for example, through a linker, to the 6-position of the compound of formula I and a residue of another cardiovascular agent can conveniently be linked, through a linker, to a residue of the b-, d- or e-carboxamide of the compound of formula I.

The invention also provides compounds having more than one cardiovascular agent attached to a compound of formula I, either directly or through a linker. For example, the residue of a cardiovascular agent can conveniently be linked, either directly or through a linker, to a residue of the b-carboxamide of the compound of formula I and a residue of another cardiovascular agent can conveniently be linked, either directly or through a linker, to a residue of the d- or e-carboxamide of the compound of formula I. In addition, the residue of a cardiovascular agent can conveniently be linked, for example, either directly or through a linker, to the 6-position of the compound of formula I and a residue of another cardiovascular agent can conveniently be linked, either directly or through a linker, to a residue of the b-, d- or e-carboxamide of the compound of formula I.

Compound of Formula I/Detectable Radionuclide or Paramagnetic Metal Atom Linkage

The invention provides compounds wherein a residue of a compound of formula I is directly linked to a detectable radionuclide (e.g. non-metallic radionuclide). A detectable radionuclide (e.g. non-metallic radionuclide) can be linked directly to any synthetically feasible position on the residue of a compound of formula I. Suitable points of attachment include, for example, the b-carboxamide, the d-carboxamide and the e-carboxamide (illustrated in FIG. 1), as well as the 6-position (the position occupied by X in FIG. 1) and the 5′-hydroxy and the 3′-hydroxy groups on the 5-membered sugar ring, although other points of attachment are possible. U.S. Pat. No. 5,739,313 discloses compounds (e.g. cyanocobalamin-b-(4-aminobutyl)amide, methylcobalamin-b-(4-aminobutyl)amide and adenosylcobalamin-b-(4-aminobutyl)amide) that are useful intermediates for the preparation of compounds of the present invention.

The invention also provides compounds having more than one detectable radionuclides (e.g. non-metallic radionuclides) directly linked to a compound of formula I. For example, the detectable radionuclide (e.g. non-metallic radionuclide) can be directly linked to a residue of the b-carboxamide of the compound of formula I and another detectable radionuclide (e.g. non-metallic radionuclide) can be directly linked to a residue of the d- or e-carboxamide of the compound of formula I. In addition, the detectable radionuclide (e.g. non-metallic radionuclide) can be directly linked to the 6-position of the compound of formula I and another detectable radionuclide (e.g. non-metallic radionuclide) can be directly linked, for example, to a residue of the b-, d- or e-carboxamide of the compound of formula I.

Compound of Formula I/Linker/Detectable Radionuclide or Paramagnetic Atom

When a detectable radionuclide (e.g. metallic radionuclide) is linked to the residue of a compound of formula I by a suitable linker, the structure of the linker is not crucial, provided it provides a compound of the invention which has an effective therapeutic and/or diagnostic index against the target cells and which will localize in or near the cardiovascular system.

Suitable linkers include linkers that separate the residue of a compound of formula I and the detectable radionuclide by about 5 angstroms to about 200 angstroms, inclusive, in length. Other suitable linkers include linkers that separate the residue of a compound of formula I and the detectable radionuclide by about 5 angstroms to about 100 angstroms, as well as linkers that separate the residue of a compound of formula I and the detectable radionuclide by about 5 angstroms to about 50 angstroms or by about 5 angstroms to about 25 angstroms. Suitable linkers are disclosed, for example, in U.S. Pat. No. 5,735,313.

The linker can conveniently be linked to the residue of a compound of formula I through an amide (e.g. —N(R)C(═O)— or —C(═O)N(R)—), ester (e.g. —OC(═O)— or —C(═O)O—), ether (e.g. —O—), ketone (e.g. —C(═O)—), thioether (eg. —S—), sulfinyl (e.g. —S(O)—), sulfonyl (e.g. —S(O)₂—) or a direct (e.g. C—C bond) linkage, wherein each R is independently H or (C₁-C₁₄)alkyl. Such a linkage can be formed from suitably functionalized starting materials using synthetic procedures that are known in the art. Based on the linkage that is desired, one skilled in the art can select suitably functional starting materials that can be derived from a residue of a compound of formula I and from a given linker using procedures that are known in the art.

The linker can be directly linked to any synthetically feasible position on the residue of a compound of formula I. Suitable points of attachment include, for example, the b-carboxamide, the d-carboxamide and the e-carboxamide (illustrated in FIG. 1), as well as the 6-position (the position occupied by X in FIG. 1) and the 5′-hydroxy and the 3′-hydroxy groups on the 5-membered sugar ring, although other points of attachment are possible. U.S. Pat. No. 5,739,313 discloses compounds (e.g. cyanocobalamin-b-(4-aminobutyl)amide, methylcobalamin-b-(4-aminobutyl)amide and adenosylcobalamin-b-(4-aminobutyl)amide) that are useful intermediates for the preparation of compounds of the present invention.

The invention also provides compounds having more than one linker attached to a compound of formula I. For example, the linker can be linked to a residue of the b-carboxamide of the compound of formula I and another linker can be directly linked to a residue of the d-carboxamide of the compound of formula I.

Specifically, the linker can comprise about 1 to about 20 detectable radionuclides. More specifically, the linker can comprise about 1 to about 10 detectable radionuclides or about 1 to about 5 detectable radionuclides.

Specifically, the linker can be a divalent radical of the formula W-A wherein A is (C₁-C₆)alkyl, (C₂-C₆)alkenyl, (C₂-C₆)alkynyl, (C₃-C₅)cycloalkyl or (C₆-C₁₀)aryl, wherein W is —N(R)C(═O)—, —C(═O)N(R)—, —OC(═O)—, —C(═O)O—, —O—, —S—, —S(O)—, —S(O)₂—, —N(R)—, —C(═O)— or a direct bond; wherein each R is independently H or (C₁-C₆)alkyl; wherein A is linked to one or more non-metallic radionuclides.

Specifically, the linker can be an amino acid or a peptide. Specifically, the peptide can be poly-L-lysine, poly-L-glutamic acid, poly-L-aspartic acid, poly-L-histidine, poly-L-omithine, poly-L-serine, poly-L-threonine, poly-L-tyrosine, poly-L-leucine, poly-L-lysine-L-phenylalanine or poly-L-lysine-L-tyrosine.

Specifically, the linker can be a chelating group capable of chelating one or more detectable radionuclides (e.g. metallic radionuclides) or paramagnetic atoms. More specifically, the linker can be a detectable chelating group.

X. Therapeutic and Diagnostic Compositions and Administrations

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate and α-glycerophosphate. Suitable inorganic salts may also be formed, including, sulfate, nitrate, bicarbonate and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Preferred modes of administration of the TC- or IF-binding agents and imaging agents are parenteral, intravenous, intradermal, intra-articular, intra-synovial, intrathecal, intra-arterial, intracardiac, intramuscular, subcutaneous, intraorbital, intracapsular, intraspinal, intrasternal, topical, transdermal patch, via rectal, vaginal or urethral suppository, peritoneal, percutaneous, nasal spray, surgical implant, internal surgical paint, infusion pump or via catheter. In one embodiment, the agent and carrier are administered in a slow release formulation such as an implant, bolus, microparticle, microsphere, nanoparticle or nanosphere. For standard information on pharmaceutical formulations, see Ansel, et al., Pharmaceutical Dosage Forms and Drug Delivery Systems, Sixth Edition, Williams & Wilkins (1995).

The TC- or IF-binding agents/imaging agents can, for example, be administered intravenously or intraperitoneally by infusion or injection. Solutions of the substance can be prepared in water, optionally mixed with a nontoxic surfactant. Dispersions can also be prepared in glycerol, liquid polyethylene glycols, triacetin and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the substance which are adapted for the extemporaneous preparation of sterile injectable or infusible solutions or dispersions, optionally encapsulated in liposomes. hi all cases, the ultimate dosage form must be sterile, fluid and stable under the conditions of manufacture and storage. The liquid carrier or vehicle can be a solvent or liquid dispersion medium comprising, for example, water, normal saline, ethanol, a polyol (for example, glycerol, propylene glycol, liquid polyethylene glycols and the like), vegetable oils, nontoxic glyceryl esters and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, benzyl alcohol, sorbic acid, thimerosal and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the substance in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Injectable solutions are particularly advantageous for local administration of the therapeutic composition. In particular, parenchymal injection can be used to deliver the therapeutic composition directly to a tumorous growth. Intra-articular injection is a preferred alternative in cases of arthritis where the practitioner wishes to treat one or only a few (such as 2-6) joints. Additionally, the therapeutic compounds are injected directly into lesions (intra-lesion administration) in appropriate cases. Intradermal administration is an alternative for dermal lesions.

The therapeutic compound is optionally administered topically by the use of a transdermal therapeutic system (see, Barry, Dermatological Formulations, (1983) p. 181 and literature cited therein). Transdermal drug delivery (TDD) has several advantages over oral delivery. When compared to oral delivery, TDD avoids gastrointestinal drug metabolism, reduces first pass effects and provides a sustained release of drugs for up to seven days (Elias, et al. Percutaneous Absorption: Mechanisms-Methodology-Drug Delivery; Marcel Dekker, NY: 1, 1989). This method is especially useful with many therapeutic proteins which are susceptible to gastrointestinal degradation and exhibit poor gastrointestinal uptake. When compared to injections, TDD eliminates the associate pain and the possibility of infection. While such topical delivery systems have been designed largely for transdernal administration of low molecular weight drugs, by definition they are capable of percutaneous delivery. They can be readily adapted to administration of the therapeutic compounds of the invention by appropriate selection of the rate-controlling microporous membrane. Topical application can also be achieved by applying the compound of interest, in a cream, lotion, ointment or oil based carrier, directly to the skin. Typically, the concentration of therapeutic compound in a cream, lotion or oil is 1-2%.

For drug targeting to lung tissue, the therapeutic compound is formulated into a solution, suspension, aerosol or particulate dispersion appropriate for application to the pulmonary system. The therapeutic agent may be inhaled via nebulizer, inhalation capsule, inhalation aerosol, nasal solution, intratracheal as a solution via syringe or endotracheal tube as an aerosol or via as a nebulizer solution. Aerosols are prepared using an aqueous aerosol, liposomal preparation or solid particles containing the compound. A nonaqueous (e.g. fluorocarbon propellant) suspension could be used. Sonic nebulizers are preferred because they minimize exposing the therapeutic compound to shear, which can result in degradation of the compound.

Delivery of the cobalamin conjugates of the instant invention by the mucosal route also offers an attractive administration alternative. The prototype formulation for nasal solutions will contain the vitamin B₁₂ conjugate dissolved in a suitable aqueous or non-aqueous solvent such as propylene glycol, an antioxidant and aromatic oils as flavoring agents. The formulation may also contain suitable propellant(s).

For ophthalmic applications, the therapeutic compound is formulated into solutions, suspensions and ointments appropriate for use in the eye. For opthalmic formulations, see Mitra (ed.), Ophthalmic Drug Delivery Systems, Marcel Dekker, Inc., New York, New York (1993) and also Havener, W. H., Ocular Pharmacology, C.V. Mosby Co., St. Louis (1983).

Useful dosages of the compounds of formula I can be determined by comparing their in vitro activity and in vivo activity in animal models. Methods for the extrapolation of effective dosages in mice and other animals, to humans are known to the art; for example, see U.S. Pat. No. 4,938,949. The amount of the substance required for use in treatment will vary not only with the particular salt selected but also with the route of administration, the nature of the condition being treated and the age and condition of the patient and will be ultimately at the discretion of the attendant physician or clinician.

In general, however, a suitable dose for nuclear medicine (using a radioactive imaging agent) will be in the range of from about 0.1 μg/patient to about 1000 μg/patient, from about 0.5 to about 500 μg/patient or from 1 μg/patient to about 100 μg/patient.

A suitable dose for imaging medicine (using a paramagnetic imaging agent) will be in the range of from about 0.1 mg/patient to about 100 mg/patient, from about 0.5 to about 50 mg/patient or from 1 mg/patient to about 10 mg/patient.

For therapeutic applications, a suitable dose will be in the range of from about 0.05 picograms/kilogram to about 100 mg/kg, from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day. The substance is conveniently administered in unit dosage form; for example, containing 5 to 1000 mg, conveniently 10 to 750 mg, most conveniently, 50 to 500 mg of active ingredient per unit dosage form.

Ideally, the substance should be administered to achieve peak plasma concentrations of from about 0.05 to about 100 μM, preferably, about I to 50 μM, most preferably, about 2 to about 30 μM. This may be achieved, for example, by the intravenous injection of a 0.005 to 10% solution of the substance, optionally in saline or orally administered as a bolus containing about 0.5-250 mg of the substance. Desirable blood levels may be maintained by continuous infusion to provide about 0.01-5.0 mg/kg/hr or by intermiftent infusions containing about 0.4-15 mg/kg of the substance.

The substance may conveniently be presented in a single dose or as divided doses administered at appropriate intervals, for example, as two, three, four or more sub-doses per day.

The cobalamin conjugates may be administered orally in combination with a pharmaceutically acceptable vehicle such as an inert diluent or an edible carrier. They may be enclosed in hard or soft shell gelatin capsules, may be compressed into tablets or may be incorporated directly with the food of the patient's diet. For oral therapeutic administration, the substance may be combined with one or more excipients and used in the form of ingestible tablets, buccal tablets, troches, capsules, elixirs, suspensions, syrups, wafers and the like. Such compositions and preparations should contain at least 0.1% of the substance. The percentage of the compositions and preparations may, of course, be varied and may conveniently be between about 2 to about 60% of the weight of a given unit dosage form. The amount of substance in such therapeutically useful compositions is such that an effective dosage level will be obtained.

Tablets, troches, pills, capsules and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the substance may be incorporated into sustained-release preparations and devices.

Sublingual tablets are designed to dissolve very rapidly. Examples of such formulations include ergotamine tartrate, isosorbide dinitrate, isoproterenol HCl. The formulation of these tablets contain, in addition to the drug, a limited number of soluble excipients, usually lactose and powdered sucrose, but occasionally dextrose and mannitol. The process of making sublingual tablets involves moistening the blended powder components with an alcohol-water solvent system containing approximately 60% alcohol and 40% water.

In addition to the cobalamin conjugate, the prototype formulation for sublingual tablets may contain a binder such as povidone or HPMC, diluents such as lactose, mannitol, starch or cellulose, a disintegrant such as pregelatinized or modified starch, lubricants such as magnesium stearate, stearic acid or hydrogenated vegetable oil, a sweetener such as saccharin or sucrose and suitable flavoring and coloring agents.

XI. Controlled Release Formulations

The TC- or IF-binding agent and imaging agent is optionally administered in a controlled release formulation, which can be a degradable or nondegradable polymer, hydrogel, organogel or other physical construct that modifies the bioabsorption, half life or biodegradation of the TC- or IF-binding agent/imaging agent. The controlled release formulation can be a material that is painted or otherwise applied onto the afflicted site, either internally or externally. In one embodiment, the invention provides a biodegradable bolus or implant that is inserted into the pocket created by surgical resection of a tumor or directly into the tumor itself. In another example, the controlled release formulation can be applied to a psoriatic lesion, eczema, atopic dermatitis, lichen planus, wart, pemphigus vulgaris, actinic keratosis, basal cell carcinoma or squamous cell carcinoma. The controlled release formulation can likewise be applied to a blood vessel to treat or prevent restenosis, retinopathies or atherosclerosis. The controlled release formulation with appropriated selected imaging agent can be used to coat a transplanted organ or tissue to prevent rejection. It can alternatively be implanted or otherwise applied near the site of rheumatoid arthritis.

The field of biodegradable polymers has developed rapidly since the synthesis and biodegradability of polylactic acid was first reported in 1966 by Kulkarni et al. “Polylactic acid for surgical implants,” Arch. Sure., 93, 839. Several other polymers are now known to biodegrade, such as polyanhydrides and polyorthoesters, which take advantage of labile backbone linkages (see: Domb et al. Macromolecules, 22, 3200, 1989; and Heller et al. Biodegradable Polymers as Drug DeliverM Systems, Dekker, NY: 1990). Several polymers which degrade into naturally occurring materials have also been described, such as crosslinking gelatin, hyaluronic acid (della Valle et al. U.S. Pat. No. 4,987,744 and U.S. Pat. No. 4,957,744) and polyaminoacids (Miyake et al., 1974), which spurred the usage of polyesters by Holland et al. Controlled Release, 4, 155, 1986 and alph-hydroxy acids (i.e. lactic acid and glycolic acid), which remain the most widely used biodegradable materials for applications ranging from closure devices (sutures and staples) to drug delivery systems (Smith et al. U.S. Pat. No. 4,741,337; Spilizeqski et al. J. Control. Rel., 2, 197, 1985).

These polymers can be tailored to degrade at a desired rate and with a desired kinetics by selecting the appropriate monomers, method of preparation and molecular weight. Differences in crystallinity of the monomer can alter the polymeric degradation rate. Due to the relatively hydrophobic nature of most polymers, actual mass loss can begin with the oligomeric fragments that are small enough to be water soluble; hence, even the initial molecular weight can influence the degradation rate.

Hydrogels can be used in controlled release formulations. Such polymers are formed from macromers with a polymerizable, non-degradable, region which is separated by at least one degradable region. For example, the water soluble, non-degradable, region can form the central core of the macromer and have at least two degradable regions which are attached to the core, such that upon degradation, the non-degradable regions (in particular a polymerized gel) are separated. Specifically, as disclosed in U.S. Pat. No. 5,626,863 to Hubbell et al., the macromers are PEG-oligoglycolyl-acrylates, with the appropriate end caps to permit rapid polymerization and gelation. Acrylates can be polymerized readily by several initiating systems such as eosin dye, ultraviolet or visible light. The polyethyleneglycol (PEG) is highly hydrophilic and biocompatible. The oligoglycolic acid is a poly(a-hydroxy acid) which can be readily degraded by hydrolysis of the ester linkage into glycolic acid, a nontoxic metabolite. Other chain extensions include polylactic acid, polycaprolactone, polyorthoesters, polyanhydrides and polypeptides. This entire network can be gelled into a biodegradable network that can be used to entrap and homogeneously disperse water-soluble drugs for delivery at a controlled rate. Further, the gel can entrap particulate suspensions of water-insoluble drugs. (See also: U.S. Pat. No. 4,591,496 to Cohen et al. (Process for Making Systems for the Controlled Release of Macromolecules); U.S. Pat. No. 5,545,442 to Van Savage et al. (Method for Using a Radiation Cured Drug Release Controlling Membrane); U.S. Pat. No. 5,330,768 to Park et al. (Controlled Drug Delivery Using Polymer/Pluronic Blends); U.S. Pat. No. 5,122,367 to Ron et al. (Polyanhydride Bioerodible Controlled Release Implants for Administration of Stabilized Growth Hormone); U.S. Pat. No. 5,545,409 to Laurencin et al. (Delivery System for Controlled Release of Bioactive Factors); U.S. Pat. No. 5,629,009 to Laurencin et al. (Delivery System for Controlled Release of Bioactive Factors).

Alternatively, delivery of biologically active substances, both in vitro and in vivo, via encapsulation has been well described in the prior art. U.S. Pat. No. 4,352,883 to Lim et al. entitled “Encapsulation of Biological Material” discloses the encapsulation of proteins within a membrane by suspending the protein in an aqueous medium containing a water-soluble gum that can be reversibly gelled to form the suspension into droplets. These droplets can be gelled further into discrete, shape-retaining, water insoluble temporary capsules with the aid of a solution of multivalent cations. The temporary capsules then can be further wrapped by an ionically cross-linking surface layer to form a semipermeable membrane around the capsules that is permeable to small molecules but impermeable to larger molecules. Microencapsulations of glycoproteins have also been well described. U.S. Pat. No. 4,324,683 to Lim et al. entitled “Encapsulation of Labile Biological Material” encapsulates a glycoprotein by a two-step interfacial polymerization process to form capsules with well-controlled porosity. The microcapsules serve to protect the active substances from attack by microorganisms and from any immunological response. U.S. Pat. No. 5,718,921 to Mathiowitz et al. (Microspheres Comprising Polymer and Drug Dispersed There Within) discloses a method to encapsulate relatively temperature-labile drugs into a microsphere.

Several methods have been developed to reversibly encapsulate biologically active substances. One that can be applied both to in vitro and in vivo studies has been described in U.S. Pat. No. 4,900,556 by Wheatley et al. entitled “System for Delayed and Pulsed Release of Biologically-Active Substances.” In this disclosed system, the biologically-active substance can be released either at a constant rate over a period of time or in discrete pulses. The biologically active materials are entrapped within liposomes encapsulated within semipermeable microcapsules or permeable polymeric matrix. Release of the desired materials is governed by the permeability of both the liposome and the surrounding matrix (the matrix integrity is directly proportional to the liposome integrity); the permeability of the liposome can be engineered by modifying the composition and the method for making the liposome to produce liposome that are sensitive to specific stimuli such as temperature, pH or light. For example, by including a phospholipase which degrades the liposome within some or all of the liposomes or the surrounding matrix, the liposome can be destabilized and broken down over a period of time. Other systems have been developed, e.g. U.S. Pat. No. 4,933,185 by Wheatley et al., which utilize a core made up of a polymer (such as an ionically cross-linked polysaccharide with calcium alginate or chitin) around which there is an ionically bound skin (such as a polycationic skin of poly-L-lysine) whose integrity is dependent on the core polymer. With an impermeable skin, when the core polymer can be degraded by enzymes (such as alginase from the bacteria, chitinase or hydrolase), there is a sudden release of biologically active substance from the core. Alternatively, the skin can be partially permeable for a gradual release of drug upon degradation of the core.

Nanoparticles are especially useful in the delivery of drugs parenterally or intravenously such that the delivery device is small with a long circulating half-life. A number of injectable drug delivery systems have been investigated, including microcapsules, microparticles, liposomes and emulsions. The major obstacle for these delivery systems is the rapid clearance of the materials from the blood stream by the macrophages of the reticuloendothelial system (RES). For example, polystyrene particles as small as sixty nanometers in diameter are cleared from the blood within two to three minutes. Liposomal drug delivery systems have also been extensively studied for this application because they were expected to freely circulate in the blood. Coating of the liposomes with poly(ethylene glycol) (PEG) increased the half-life of the carriers due to PEG's hydrophobic chains which reduced its protein absorption and thus its RES uptake. U.S. Pat. No. 5,543,158 to Gref et al. (Biodegradable Injectable Nanoparticles) describes a carrier system specifically targeted towards carriers suitable for intravenous delivery with a controlled release mechanism with modified polyglycols.

U.S. Pat. No. 5,626,862, U.S. Pat. No. 5,651,986 and U.S. Pat. No. 5,846,565 to Brem et al. (Controlled Local Delivery of Chemotherapeutic Agents for Treating Solid Tumors) discloses the use of these carriers for the specific delivery of chemotherapeutic agents to increase bioavailability. Therefore, the devices act as reservoirs that release drugs over an extended period of time while at the same time preserves the bioactivity and bioavailability of the agent. U.S. Pat. No. 5,286,763 to Gerhard et al. (Bioerodible Polymers for Drug Delivery in Bone) further discloses that bioerodible polymers can be used to deliver chemotherapeutic agents directly into the bone. Cohen et al. U.S. Pat. No. 5,562,099 (Polymeric Microparticles Containing Agents for Inaging) discusses the usage of these carriers are contrast agents. The polymeric microparticle is filled with contrast agents for enhanced imaging.

Furthermore, U.S. Pat. No. 6,114,394 to Edwards, et al. (Polyamine Derivatives as Radioprotective Agents) discloses polyamine derivatives and the pharmaceutically acceptable addition salts thereof which are useful as radioprotective agents. The potential utility of these agents in protecting against exposure to environmental radiation, as well as in cancer radiation therapy, has long bee recognized. These agents, administered prior to or during exposure, would eliminate or reduce the severity of deleterious cellular effects caused by exposure to environmental ionizing radiation such as resulting from a nuclear explosion, a spill of radioactive material, close proximity to radioactive material and the like.

Books describing methods of controlled delivery that are appropriate for the delivery of the TC- or IF-binding agents/imaging agents of the present invention include: Robert S. Langer, Donald L. Wise, editors; Medical aplications of controlled release (Volumes 1 and 2); Boca Raton, Fla.: CRC Press, 1984; and William J. M. Hrushesky, Robert Langer and Felix Theeuwes, editors; Temporal control of drug delivery (series); New York: New York Academy of Sciences, 1991.

Nonlimiting examples of U.S. Patents that describe controlled release formulations are: U.S. Pat. No. 5,356,630 to Laurencin et al. (Delivery System for Controlled Release of Bioactive Factors); ; U.S. Pat. No. 5,797,898 to Santini, Jr. et al. (Microchip Drug Delivery Devices); U.S. Pat. No. 5,874,064 to Edwards et al. (Aerodynamically Light Particles for Pulnonary Drug Delivery); U.S. Pat. No. 5,548,035 to Kim et aL (Biodegradable Copolymer as Drug Delivery Matrix Comprising Polyethyleneoxide and Aliphatic Polyester Blocks); U.S. Pat. No. 5,532,287 to Savage et al. (Radiation Cured Drug Release Controlling Membrane); U.S. Pat. No. 5,284,831 to Kahl et al. (Drug Delivery Porphyrin Composition and Methods); U.S. Pat. No. 5,741,329 to Agrawal et al. (Methods of Controlling the pH in the Vicinity of Biodegradable Implants); U.S. Pat. No. 5,820,883 to Tice et al (Methods for Delivering Bioactive Agents into and Through the Mucosally-Associated Lymphoid Tissues and Controlling Their Release);U.S. Pat. No. 5,955,068 to Gouin et al. (Biodegradable Polyanhydrides Derived from Dimers of Bile Acids and Use Thereof as Controlled Drug Release Systems); U.S. Pat. No. 6,001,395 to Coombes et al. (Polymeric Lamellar Substrate Particles for Drug Delivery); U.S. Pat. No. 6,013,853 to Athanasiou et al. (Continuous Release Polymeric Implant Carriers); U.S. Pat. No. 6,060,582 to Hubbell et al. (Photopolymerizable Biodegradable Hydrogels as Tissue Contacting Materials and Controlled Release Carriers); U.S. Pat. No. 6,113,943 to Okada et al. (Sustained-Release Preparation Capable of Releasing a Physiologically Active Substance); and PCT Publication No. WO 99/59548 to Oh et al (Controlled Drug Delivery System Using the Conjugation of Drug to Biodegradable Polyester); U.S. Pat. No. 6,123,861 (Fabrication of Microchip Drug Delivery Devices); U.S. Pat. No. 6,060,082 (Polymerized Liposomes Targeted to M cells and Useful for Oral or Mucosal Drug Delivery); U.S. Pat. No. 6,041,253 (Effect of Electric Field and Ultrasound for Transdermal Drug Delivery); U.S. Pat. No. 6,018,678 (Transdermal protein delivery or measurement using low-frequency sonophoresis); U.S. Pat. No. 6,007,845 Nanoparticles And Microparticles Of Non-Linear Hydrophilic-Hydrophobic Multiblock Copolymers; U.S. Pat. No. 6,004,534 Targeted Polymerized Liposomes For Improved Drug Delivery; U.S. Pat. No. 6,002,961 Transdermal Protein Delivery Using Low-Frequency Sonophoresis; U.S. Pat. No. 5,985,309 Preparation Of Particles For Inhalation; U.S. Pat. No. 5,947,921 Chemical And Physical Enhancers And Ultrasound For Transdermal Drug Delivery; U.S. Pat. No. 5,912,017 Multiwall Polymeric Microspheres; U.S. Pat. No. 5,911,223 Introduction Of Modifying Agents Into Skin By Electroporation; U.S. Pat. No. 5,874,064 Aerodynamically Light Particles For Pulmonary Drug Delivery; U.S. Pat. No. 5,855,913 Particles Incorporating Surfactants For Pulmonary Drug Delivery; U.S. Pat. No. 5,846,565 Controlled Local Delivery Of Chemotherapeutic Agents For Treating Solid Tumors; U.S. Pat. No. 5,837,752 Semi-Interpenetrating Polymer Networks; U.S. Pat. No. 5,814,599 Transdermal Delivery Of Encapsulated Drugs; U.S. Pat. No. 5,804,178 Implantation Of Cell-Matrix Structure Adjacent Mesentery, Omentum Or Peritoneum Tissue; U.S. Pat. No. 5,797,898 Microchip Drug Delivery Devices; U.S. Pat. No. 5,770,417 Three-Dimensional Fibrous Scaffold Containing Attached Cells For Producing Vascularized Tissue In vivo; U.S. Pat. No. 5,770,193 Preparation Of Three-Dimensional Fibrous Scaffold For Attaching Cells To Produce Vascularized Tissue In vivo; U.S. Pat. No. 5,762,904 Oral Delivery Of Vaccines Using Polymerized Liposomes; U.S. Pat. No. 5,759,830 Three-Dimensional Fibrous Scaffold Containing Attached Cells For Producing Vascularized Tissue In vivo; U.S. Pat. No. 5,749,847 Delivery Of Nucleotides Into Organisms By Electroporation; U.S. Pat. No. 5,736,372 Biodegradable Synthetic Polymeric Fibrous Matrix Containing Chondrocyte For In vivo Production Of A Cartilaginous Structure; U.S. Pat. No. 5,718,921 Microspheres Comprising Polymer And Drug Dispersed There Within; U.S. Pat. No. 5,696,175 Preparation Of Bonded Fiber Structures For Cell Implantation; U.S. Pat. No. 5,667,491 Method For Rapid Temporal Control Of Molecular Transport Across Tissue; U.S. Pat. No. 5,654,381 Functionalized Polyester Graft Copolymers; U.S. Pat. No. 5,651,986 Controlled Local Delivery Of Chemotherapeutic Agents For Treating Solid Tumors; U.S. Pat. No. 5,629,009 Delivery System For Controlled Release Of Bioactive Factors; U.S. Pat. No. 5,626,862 Controlled Local Delivery Of Chemotherapeutic Agents For Treating Solid Tumors; U.S. Pat. No. 5,593,974 Localized Oligonucleotide Therapy; U.S. Pat. No. 5,578,325 Nanoparticles And Microparticles Of Non-Linear Hydrophilic-Hydrophobic Multiblock Copolymers; U.S. Pat. No. 5,562,099 Polymeric Microparticles Containing Agents For Imaging; U.S. Pat. No. 5,545,409 Delivery System For Controlled Release Of Bioactive Factors; U.S. Pat. No. 5,543,158 Biodegradable Injectable Nanoparticles; U.S. Pat. No. 5,514,378 Biocompatible Polymer Membranes And Methods Of Preparation Of Three Dimensional Membrane Structures; U.S. Pat. No. 5,512,600 Preparation Of Bonded Fiber Structures For Cell Implantation; U.S. Pat. No. 5,500,161 Method For Making Hydrophobic Polymeric Microparticles; U.S. Pat. No. 5,487,390 Gas-filled polymeric microbubbles for ultrasound imaging; U.S. Pat. No. 5,399,665 Biodegradable polymers for cell transplantation; U.S. Pat. No. 5,356,630 Delivery system for controlled release of bioactive factors; U.S. Pat. No. 5,330,768 Controlled drug delivery using polymer/pluronic blends; U.S. Pat. No. 5,286,763 Bioerodible polymers for drug delivery in bone; U.S. Pat. No. 5,149,543 Tonically cross-linked polymeric microcapsules; U.S. Pat. No. 5,128,420 Method of making hydroxamic acid polymers from primary amide polymers; U.S. Pat. No. 5,122,367 Polyanhydride bioerodible controlled release implants for administration of stabilized growth hormone; U.S. Pat. No. 5,100,668 Controlled release systems containing heparin and growth factors; U.S. Pat. No. 5,019,379 Unsaturated polyanhydrides; U.S. Pat. No. 5,010,167 Poly(amide-and imide-co-anhydride) for biological application; U.S. Pat. No. 4,948,587 Ultrasound enhancement of transbuccal drug delivery; U.S. Pat. No. 4,946,929 Bioerodible articles useful as implants and prostheses having predictable degradation rates; U.S. Pat. No. 4,933,431 One step preparation of poly(amide-anhydride); U.S. Pat. No. 4,933,185 System for controlled release of biologically active compounds; U.S. Pat. No. 4,921,757 System for delayed and pulsed release of biologically active substances; U.S. Pat. No. 4,916,204 Pure polyanhydride from dicarboxylic acid and coupling agent; U.S. Pat. No. 4,906,474 Bioerodible polyanhydrides for controlled drug delivery; U.S. Pat. No. 4,900,556 System for delayed and pulsed release of biologically active substances; U.S. Pat. No. 4,898,734 Polymer composite for controlled release or membrane formation; U.S. Pat. No. 4,891,225 Bioerodible polyanhydrides for controlled drug delivery; U.S. Pat. No. 4,888,176 Controlled drug delivery high molecular weight polyanhydrides; U.S. Pat. No. 4,886,870 Bioerodible articles useful as implants and prostheses having predictable degradation rates; U.S. Pat. No. 4,863,735 Biodegradable polymeric drug delivery system with adjuvant activity; U.S. Pat. No. 4,863,611 Extracorporeal reactors containing immobilized species; U.S. Pat. No. 4,861,627 Preparation of multiwall polymeric microcapsules; U.S. Pat. No. 4,857,311 Polyanhydrides with improved hydrolytic degradation properties; U.S. Pat. No. 4,846,786 Bioreactor containing suspended, immobilized species; U.S. Pat. No. 4,806,621 Biocompatible, bioerodible, hydrophobic, implantable polyimino carbonate article; U.S. Pat. No. 4,789,724 Preparation of anhydride copolymers; U.S. Pat. No. 4,780,212 Ultrasound enhancement of membrane permeability; U.S. Pat. No. 4,779,806 Ultrasonically modulated polymeric devices for delivering compositions; U.S. Pat. No. 4,767,402 Ultrasound enhancement of transdermal drug delivery; U.S. Pat. No. 4,757,128 High molecular weight polyanhydride and preparation thereof; U.S. Pat. No. 4,657,543 Ultrasonically modulated polymeric devices for delivering compositions; U.S. Pat. No. 4,638,045 Non-peptide polyamino acid bioerodible polymers; U.S. Pat. No. 4,591,496 Process for making systems for the controlled release of macromolecules.

The invention may be further illustrated by the following examples.

### EXAMPLES

### Example 1

Preparation of Cyanocobalamin-b(4-aminobutyl amid

A mixture containing cyanocobalamin-b-carboxylic acid (1.0 g, 0.6 mmol), hydroxybenzotriazole (0.81 g, 6 mmol) and 1,4-diaminobutane dihydrochloride (4.8 g, 30 nnmol) in 100 ml water was adjusted to Ph 7.8. 1-Ethyl-3-(3′-dimethylaminopropyl)carbodiimide (1.26 g, 6.6. mmol) was then added, the pH was adjusted to 6.4 and the reaction stirred at room temperature for 24 h. TLC on silica gel using n-butanol-acetic water (5:2:3) showed the reaction to be complete. Cyanocobalamin-b(4-aminobutyl)amide was extracted into 92% aqueous phenol and the phenol layer was washed several times with equal volumes of water. To the phenol extract were added 3 volumes of diethylether and 1 volume of acetone. The desired cobalamin was removed from the organic phase by several extractions with water. The combined aqueous layers were extracted three times with diethylether to remove residual phenol, concentrated to approximately 20 ml in vacuo and crystallized from aqueous acetone. Yield 955 mg, 92%.

### Example 2

Proposed preparation of Cyanocobalamin-b-(4-aminobutyl)amide-lisinopril-, Fosinopril Sodium-, Enalaprilat- and Cantopril-Cobalamin Coniugates

A mixture containing cyanocobalamin-b-(4-aminobutyl)amide (0.6 nmuol), hydroxy-benzotriazole (6 mmol) and the cardiovascular agent (e.g. Lisinopril, Fosinopril Sodium, Enalaprilat or Captopril) (30 nunol) in 100 ml of water is adjusted to a pH of 7.8. 1-Ethyl-3-(3′-dimethylaminopropy)carbodiimide (6.6 mmol) is then added, the pH is adjusted to 6.4 and the reaction is stirred at room temperature for 24 h. TLC on silica gel using n-butanol-acetic acid water (5:2:3) shows when the reaction is complete. The product is extracted into 92% aqueous phenol and the phenol layer is washed several times with equal volumes of water. To the phenol extract is added 3 volumes of diethylether and 1 volume of acetone. The desired product is removed from the organic phase by several extractions with water. The combined aqueous layers are extracted three times with diethylether to remove residual phenol. concentrated to approximately 20 ml in vacuo and crystallized from aqueous acetone.

### Example 3

Preparation of Methylcobalamin-b-(4-aminobutyl)amide

Methylcobalamin-b-carboxylic acid (1.0 g, 0.6 mmol) was reacted with diaminobutane dihydrochloride as described above for the cyano derivative. The cobalamin was purified by extraction through phenol (see above) and the resulting aqueous solution was concentrated in vacuo. This solution was chromatographed on AGI-X2200-400 mesh in the acetate form (20.times.2.5. cm) and the pass through collected. The pass through was concentrated to approximately 20 ml and the desired cobalamin crystallized from aqueous acetone. Yield 920 mg, 88%. Unreacted methylcobalamin-b-carboxylic acid was eluted with 1 M acetic acid, concentrated and crystallized from aqueous acetone. Yield 60 mg, 6%.

### Example 4

Proposed Preparation of Methylcobalamin-b-(4-aminobutyl)amide-Lisinopril-, Fosinopril Sodium-, Enalaprilat- and Captopril-Cobalamin Conjugates.

A mixture containing methylcobalamin-b-(40aminobutyl)amide (0.6 mmol), hydroxy-benzotriazole (6 mmol) and the cardiovascular agent (e.g. Lisinopril-, Fosinopril Sodium-, Enalaprilat- and Captopril) (30 mmol) in 100 ml of water is adjusted to pH 7.8 1-Ethyl-3-(3′-dimethylaminopropy)carbodiimide (6.6 mmol) is then added, the pH is adjusted to 6.4 and the reaction is stirred at room temperature for 24 h. TLC on silica gel using n-butanol-acetic acid water (5:2;3) shows the reaction to be complete. The product is extracted into 92% aqueous phenol and the phenol layer is washed several times with equal volumes of water. To the phenol extract is added 3 volumes of diethylether and 1 volume of water. To the phenol extract is added 3 volumes of diethylether and 1 volume of acetone. The desired product is removed from the organic phase by several extractions with water. The combined aqueous layers are extracted three times with diethylether to remove residual phenol, concentrated to approximately 20 ml in vacuo and crystallized from aqueous acetone.

### Example 5

Preparation of Adenosylcobalamin-b(4-aminobutyl amide

Adenosylcobalamin-b-carboxylic acid (500 mg, 0.3. mmol) was reacted with diaminobutane dihydrochloride (2.4 mg, 15 mmol) as described above. The cobalamin was purified by extraction through phenol (see above). The resulting aqueous solution was concentrated in vacuo and applied to AG-50-X2, 200-400 mesh, in the hydrogen from 20.times.25 cm). The column was washed thoroughly with water to remove hydroxybenzotriazole and the desired cobalamin eluted with 1M ammonium hydroxide. After an additional extraction through phenol, adenosylcobalamin-b-(4-aminobutyl)amide was isolated as a glass. Yield 366 mg, 77%.

### Example 6

Proposed Preparation of Adenosylcobalamin-b(4-aminobutyl)amide-lisinopril-, Fosinopril Sodium-, Enalaprilat- and Captopril-Cobalamin Conjugates

A mixture containing adenosylcobalamin-b-(4-aminobutyl)amide (0.6 mmol). hydroxybenzotriazole (6 mmol) and the cardiovascular agent (e.g. Lisinopril, Fosinopril Sodium, Enalaprilat or Capotoptil) (30 mmol) in 100 ml of water is adjusted to pH 7.8. 1-Ethyl-3-)3'dimethylaminopropyl)carbodiimide (6.6 mmol) is then added, the pH is adjusted to 6.4 and the reaction is stirred at room temperature for 24 h. TLC on silica gel using n-butanol-acetic acid water (5:2:3) shows the reaction to be complete. The product is extracted into 92% aqueous phenol and the phenol layer is washed several times with equal volumes of water. To the phenol extract is added 3 volumes of diethylether and 1 volume of acetone. The desired product is removed from the organic phase by several extractions with water. The combined aqueous layers are extracted three times with diethylether to remove residual phenol, concentrated to approximately 20 ml in vacuo and crystallized from aqueous acetone.

### Example 7

Preparation of Cyanocobalamin-b-(poly-L-lysine)amide-

Two preparations of -poly-1-lysine hydrobromide, one containing approximately 8 residues and a second one containing about 11 residues were separately reacted with cyanocobalamin-1-carboxylic acid. To each polymer (500 mg) dissolved in 20 mL of water was added 150 mg (0.1 mmol) or cyanocobalamin-1-carboxylic acid, 338 mg (2.5 mmol) of hydroxybenzotriazole and 480 mg (2.5 mmol) of 1 Oethyl-3(3-dimethyl-aminopropyl) carbodiimide. The pH was adjusted to 9 with IN NaOH and the reaction mixtures were stirred at room temperature for 2-3 h. They were purified on G-10 sephadex: the sizing columns (3×40 cm) were eluded with water and 1.5 mL fractions collected. The fractions showing the presence of the cobalamin (OD at 550 mm) and the presence of polylysine (ninhydrin positive) were pooled and freeze-dried.

### Example 8

Proposed Preparation of Cvanocobalamin-b-(polyl sine)amide-lisinopril-, Fosinopril Sodium-, Enalaprilat- and Captopril-Conjuates.

A mixture containing cyanocobalamin-b-(polylysine)amide, hydroxybenzotriazole (0.81 g, 6 mmol) and the cardiovascular agent (e.g. Lisinopril, Fosinopril Sodium, Enalaprilat or Captopril) (30 mmol) in 100 ml of water is adjusted to pH 7.8. 1-Ethyl-3-(3′-dimethylaminopropyl)carbodiimide (1.26 g, 6.6 mmol) is then added, the pH is adjusted to 6.4 and the reaction is stirred at room temperature for 24 h. TLC on silica gel using n-butanol-acetic acid water (5:2:3) shows the reaction to be complete. The reaction mixture is purified on G-10 sephadex: the sizing columns 3×40 cm) are eluted with water and 1.5 mL fractions collected. The fractions showing the presence of the cobalamin (OD at 550 mm) and the presence of polylysine (ninhydrin positive) are pooled and freeze-dried.

All publications, patents and patent documents are incorporated by reference herein, as though individually incorporated by reference. The invention has been described with reference to various specific and preferred embodiments and techniques. However, it should be understood that many variations and modifications may be made while remaining within the spirit and scope of the invention. In addition, some references were obtained on the world wide web (www). These references have been reproduced and are enclosed herein, as pages 53-125. These references are also incorporated by reference herein, as though individually incorporated by reference.

We claim:

1. A compound of the formula I: 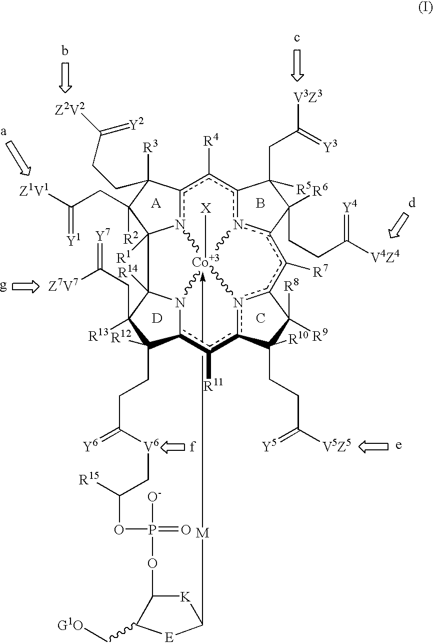
or its pharmaceutically acceptable salt, wherein:
a) the wavy line in the chemical structure indicates either a dative or covalent bond such that there are three dative Co—N bonds and one covalent Co—N bond, wherein, in the case of the dative bond, the valence of nitrogen is completed either with a double bond with an adjacent ring carbon or with a hydrogen;
b) the dotted line in the chemical structure indicates either a double or single bond such that the double bond does not over-extend the valence of the element (i.e. to give pentavalent carbons) and, in the case of a single bond, the valence is completed with hydrogen;
c) X is hydrogen, cyano, halogen (Cl, F, Br or I), haloalkyl, CF₃, CF₂CF₃, CH₂CF₃, CF₂Cl, NO, NO₂, NO₃, phosphonate, alkyl-P(P)₂OR¹⁵), PR¹⁵R¹⁶R¹⁷, N₂, NR¹⁵R¹⁶, OH, OR¹⁵, SR¹⁵, SCN, N₃, OC(O)R¹⁵, C(O)₂R¹⁵, C(O)R¹⁵, OC(O)NR¹⁵R¹⁶, C(o)₂NR¹⁵R¹⁶, C(o)N¹⁵R¹⁶, P(O)₂OR¹⁵, S(O)₂ OR¹⁵, a purine or pyrimidine nucleoside or nucleoside analog, adenosyl, 5-FU, alkyl, alkenyl, alkynyl, aryl, aralkyl, alkaryl, amino acid, peptide, protein, carbohydrate, heteroalkyl, heterocycle, heteroaryl, alkylheteroaryl or L-T;
d) M is a monovalent heterocycle or heteroaromatic, which is capable of binding to the adjacent sugar ring;
e) K is O, S, NJ¹, C(OH)H, CR¹⁰⁰R¹⁰¹ or C(R¹⁰⁰)V⁸Z⁸;
f) E is O or S;
g) G¹ is hydrogen, alkyl, acyl, silyl, mono-, di- or tri-phosphate or L-T;
h) Y¹, Y², Y³, Y⁴, Y⁵, Y⁶ and Y⁷ independently are O, S or NJ²; direct bond;
j) Z¹, Z², Z³, Z⁴, Z⁵, Z⁷ and Z⁸ independently are R¹⁰⁴ or L-T;
k) each L is independently a direct bond or a linker to one or more T moieties and that does not significantly impair the ability of the TC- or IF-binding agent to bind to a transcobalamin receptor;
l) each T independently comprises a cardiovascular agent, or pharmaceutically acceptable residue thereof, optionally bound though a chelating moiety;
m) wherein at least one of Z¹, Z², Z³, Z⁴, Z⁵, Z⁷, Z⁸ and G¹ is L-T;
n) J¹, J² and J³ independently are hydrogen, alkyl, alkenyl, alkynyl, alkaryl, cycloalkyl, aryl, cycloaryl, heteroalkyl, heterocycle, heteroaryl, hydroxyl, alkoxy or amine;
o) R¹, R, R³, R⁴, R⁵, R⁶, R⁷, R⁸, R⁹, R¹⁰, R¹¹, R¹², R¹³ and R¹⁴ independently are hydrogen, lower alkyl, lower alkenyl, lower alkynyl, lower cycloalkyl, heteroalkyl, heterocyclic, lower alkoxy, azido, amino, lower alkylamino, halogen, thiol, SO₂, SO₃, carboxylic acid, C₁-6 carboxyl, hydroxyl, nitro, cyano, oxime or hydrazine;
p) R¹³ and R¹⁴ optionally can form a double bond;
q) R¹⁵, R¹⁶ and R¹⁷ are independently hydrogen, alkyl, alkenyl, alkynyl, aryl, alkaryl or aralkyl group, heteroalkyl, heterocycle or heteroaromatic; and
r) R¹⁰⁰, R^{101, R}¹⁰², R¹⁰³ and R¹⁰⁴ are independently hydrogen, alkyl, alkenyl, alkynyl, aryl, acyl, heteroaromatic, heteroaryl, heteroalkyl, hydroxyl, alkoxy, cyano, azido, halogen, nitro, SO₂, SO₃, thioalkyl or amino.

2. The compound of claim 1 wherein
a) X is CN, OH, CH₃, adenosyl or L-T;
b) M is 5,6-dimethylbenzimidazole;
c) K is C(OH)H;
d) E is O;
e) G¹ is hydrogen, alkyl, acyl, silyl, mono-, di- or tri-phosphate or L-T
f) Y¹, Y², Y³, Y⁴, Y⁵, Y⁶ and Y⁷ are O
g) V¹, V², V³, V⁴, V⁵, V⁶, V⁷ and V⁸ are independently NJ³;
h) R¹, R², R⁴, R⁵, R⁸, R⁹, R¹¹, R¹² and R¹⁵ are independently methyl;
i) R³, R⁶, R⁷, R¹⁰, R¹³ and R¹⁴ are independently hydrogen; and
j) Z¹, Z², Z³, Z⁴, Z⁵, Z⁷ and Z⁸ are independently hydrogen or L-T.

3. The compound of claim 1 wherein, M is a purine or pyrimidine.

4. The compound of claim 1 wherein M is 5,6-dimethylbenzimidazole.

5. The compound of claims 1 wherein, X is not L-T.

6. The compound of claim 1 wherein at least one of Z¹, Z², Z⁴ or Z⁵ is independently L-T.

7. The compound of claim 1 wherein at least two of Z¹, Z², Z⁴ or Z⁵ are independently L-T.

8. The compound of claim 6 or 7 wherein L is a bond.

9. The compound of claim 6 or 7 wherein L is not a bond.

10. The compound of claim 9 wherein at least one L is of the formula W-A-Q wherein A is (C₁-C₂₄)alkyl, (C₂-C₂₄)alkenyl, (C₂-C₂₄)alkynyl, (C₃-C₈)cycloalkyl, or (C6-C₁₀)aryl, wherein W and Q are each independently —N(R)C(═O)—, —C(═O)N(R)—, —OC(═O)—, —C(═O)O—, —O—, —S—, —S(O)—, —S(O)₂—, —N(R)—, —C(═O)—, or a direct bond; wherein each R is independently H or (C₁-C₆)alkyl.

11. The compound of claim 10 wherein at least one of W and Q is independently —NR— or —COO—.

12. The compound of claim 10 wherein A is a (C₁-C₂₄)alkyl.

13. The compound of claim 9 wherein at least one L is about 5 angstroms to about 50 angstroms, in length, inclusive.

14. The compound of claim 9 wherein at least one L is a divalent radical formed from a peptide.

15. The compound of claim 9 wherein at least one L is a divalent radical formed from about 2-25 amino acids.

16. The compound of claim 14 or 15 wherein the divalent radical is a 1,ω-divalent radical.

17. The compound of claim 9 wherein at least one L is poly-L-glutamic acid, poly-L-aspartic acid, poly-L-histidine, poly-L-ornithine, poly-L-serine, poly-L-threonine, poly-L-tyrosine, poly-L-leucine, poly-L-lysine-L-phenylalanine, poly-L-lysine or poly-L-lysine-L-tyrosine.

18. The compound of claim 17 wherein L is poly-L-lysine.

19. The compound of claim 18 wherein the poly-L-lysine contains about 8-11 residues.

20. The compound of claim 6 or 7 wherein Z′ is L-T.

21. The compound of claim 6 or 7 wherein Z² is L-T.

22. The compound of claim 6 or 7 wherein Z⁴ is L-T.

23. The compound of claim 6 or 7 wherein Z⁵ is L-T.

24. The compound of claim 6 or 7 wherein Z² and Z⁴ are independently L-T.

25. The compound of claim 1 wherein at least one T is independently a blood modifier, adrenergic blocker, adrenergic stimulant, alpha/beta adrenergic blocker, angiotensin converting enzyme inhibitor, angiotensin II receptor antagonist, group I anti-arrhythmic, group II anti-arrhythmic, group Im anti-arrhythmic, group IV anti-arrhythmic, miscellaneous anti-arrhythmic, anti-lipemic agent, beta adrenergic blocking agent, calcium channel blocker, diuretic, hypertensive emergency agent, inotropic agent, miscellaneous cardiovascular agent, rauwolfia derivative, vasodilator or vasopressor, or a pharmaceutically acceptable residue thereof.

26. The compound of claim 1 wherein at least one T is independently Coumadin, Fragmin, Heparin Lock, Heparin sodium, Lovenox, Normiflo, Orgaran, Aggrastat, Agrylin, Ecotrin, Flolan, Halfprin, Integrilin, Persantine, Plavix, ReoPro, Ticlild, Neupogen, Leukine, Anadrol-50, Nascobal, Trinsicon, Epogen, Procrit, Cardura, Dibenzyline, Hylorel, Hytrin, Minipress, Minizide, Aldoclor, Aldomet, Aldomet ester HCl, Aldoril, Catapres, Catapres-TTS, Clorpres, Combipers, Tenex, Coreg, Normodyne, Trandate, Accupril, Altace, Captopril, Lotensin, Mavik, Monopril, Prinivil, Univasc, Vasotec, Zestril, Atacand, Avapro, Cozaar, Diovan, Cadrioquin, Ethmozine, Mexitil, Norpace, Norpace CR, Procanbid, Quinaglute, Quinidex, Rythmol, Tambocor, Tonocard, Betapace, Brevibloc, Inderal, Sectral, Cordarone, Corvert, Pacerone, Calan, Cardizem, Adenocard, Lanoxicaps, Lanoxin, Colestid, LoCholest, Questran, Atromid-S, Lopid, Tricor, Baycol, Lescol, Lipitor, Mevacor, Pravachol, Zocor, Niaspan, Blocadren, Brevibloc, Cartrol, Inderal, Kerlone, Levatol, Lopressor, Sectral, Tenormin, Toprol-XL, Zebeta, Adalat, Adalat CC, Calan, Calan SR, Cardene, Cardizem CD, Cardizem, Cardizem SR, Covera-HS, Dilacor XR, DynaCirc, DynaCirc CR, Isoptin SR, Nimotop, Norvasc, Plendil, Procardia, Procardia XL, Sular, Tiazac, Vascor, Verelan, Daranide, Demadex, Edecrin, Edecrin sodium, Lasix, Aldactone, Dyrenium, Midamor, Diucardin, Diuril, Diuril sodium, Enduron, HydroDIURIL, Microzide, Mykrox, Renese, Thalitone, Zaroxolyn, Hyperstat, Dobutrex, Lanoxicaps, Lanoxin, Primacor, Demser, Inversine, Regitine, ReoPro, Diupres, Hydropres, Deponit, Dilatrate-SR, lIndur, Ismo, Isordil, Monoket, Nitro-Bid, Nitro-Dur, Nitrolingual, Nitrostat, Sorbitrate, Transderm-Nitro, Corlopam, Flolan, Primacor, Ana-Kit, Aramine, EpiPen, ProAmatine or Vasoxyl, or a pharmaceutically acceptable residue thereof.

27. The compound of claim 1 wherein at least one T is independently Lisinopril, Fosinopril Sodium, Enalaprilat or Captopril, or a pharmaceutically acceptable residue thereof.

28. The compound of claim 1 wherein the Z¹, Z², Z³, Z⁴, Z⁵, Z⁷, Z⁸ or G¹ moiety that is not L-T can further independently be L-T′ wherein T′ is an imaging agent, optionally bound through a chelating moiety.

29. The compound of claim 28 wherein the imaging agent is bound through a chelating moiety.

30. The compound of claim 29 wherein the chelating moiety is DPTA.

31. The compound of claim 29 wherein the imaging agent is a detectable radionuclide or a paramagnetic metal atom.

32. The compound of claim 31 wherein the detectable radionuclide or a paramagnetic metal atom is Technetium-99m, Indium-111 or Gadolinium-157.

33. The compound of claim 28, wherein the imaging agent is not bound through a chelating moiety.

34. The compound of claim 28 wherein the imaging agent is a non-metallic radionuclide.

35. The compound of claim 34 wherein the non-metallic radionuclide is carbon-11, fluorine-18, bromine-76, iodine-123 or iodine-124.

36. The compound of claim 28, wherein at least one of Z¹, Z², Z⁴ or Z⁵ is not L-T and is independently L-T′, wherein T′ is the imaging agent.

37. The compound of claim 37 wherein L is a bond.

38. The compound of claim 37 wherein L is not a bond.

39. A pharmaceutical composition for the treatment, prophylaxis or diagnosis of a cardiovascular disease in a host comprising the compound of any one of the preceding claims 1-38, or its pharmaceutically acceptable salt, together with a pharmaceutically acceptable carrier or diluent.

40. A pharmaceutical composition for the treatment, prophylaxis or diagnosis of an cardiovascular disease in a host comprising the compound of any one of the preceding claims 1-38, or its pharmaceutically acceptable salt, in combination with one or more cardiovascular agent.

41. The composition of claim 40 wherein the cardiovascular agent is a blood modifier, adrenergic blocker, adrenergic stimulant, alpha/beta adrenergic blocker, angiotensin converting enzyme inhibitor, angiotensin II receptor antagonist, group I anti-arrhythmic, group II anti-arrhythmic, group mi anti-arrhythmic, group IV anti-arrhythmic, miscellaneous anti-arrhythmic, anti-lipemic agent, beta adrenergic blocking agent, calcium channel blocker, diuretic, hypertensive emergency agent, inotropic agent, miscellaneous cardiovascular agent, rauwolfia derivative, vasodilator or vasopressor, or a pharmaceutically acceptable residue thereof.

42. The composition of claim 40 wherein the cardiovascular agent is Coumadin, Fragmin, Heparin Lock, Heparin sodium, Lovenox, Normiflo, Orgaran, Aggrastat, Agrylin, Ecotrin, Flolan, Halfprin, Integrilin, Persantine, Plavix, ReoPro, Ticlild, Neupogen, Leukine, Anadrol-50, Nascobal, Trinsicon, Epogen, Procrit, Cardura, Dibenzyline, Hylorel, Hytrin, Minipress, Minizide, Aldoclor, Aldomet, Aldomet ester HCl, Aldoril, Catapres, Catapres-TTS, Clorpres, Combipers, Tenex, Coreg, Normodyne, Trandate, Accupril, Altace, Captopril, Lotensin, Mavik, Monopril, Prinivil, Univasc, Vasotec, Zestril, Atacand, Avapro, Cozaar, Diovan, Cadrioquin, Ethmozine, Mexitil, Norpace, Norpace CR, Procanbid, Quinaglute, Quinidex, Rythmol, Tambocor, Tonocard, Betapace, Brevibloc, Inderal, Sectral, Cordarone, Corvert, Pacerone, Calan, Cardizem, Adenocard, Lanoxicaps, Lanoxin, Colestid, LoCholest, Questran, Atromid-S, Lopid, Tricor, Baycol, Lescol, Lipitor, Mevacor, Pravachol, Zocor, Niaspan, Blocadren, Brevibloc, Cartrol, Inderal, Kerlone, Levatol, Lopressor, Sectral, Tenormin, Toprol-XL, Zebeta, Adalat, Adalat CC, Calan, Calan SR, Cardene, Cardizem CD, Cardizem, Cardizem SR, Covera-HS, Dilacor XR, DynaCirc, DynaCirc CR, Isoptin SR, Nimotop, Norvasc, Plendil, Procardia, Procardia XL, Sular, Tiazac, Vascor, Verelan, Daranide, Demadex, Edecrin, Edecrin sodium, Lasix, Aldactone, Dyrenium, Midamor, Diucardin, Diuril, Diuril sodium, Enduron, HydroDRJRIL, Microzide, Mykrox, Renese, Thalitone, Zaroxolyn, Hyperstat, Dobutrex, Lanoxicaps, Lanoxin, Primacor, Demser, Inversine, Regitine, ReoPro, Diupres, Hydropres, Deponit, Dilatrate-SR, hndur, Ismo, Isordil, Monoket, Nitro-Bid, Nitro-Dur, Nitrolingual, Nitrostat, Sorbitrate, Transderm-Nitro, Corlopam, Flolan, Primacor, Ana-Kit, Aramine, EpiPen, ProAmatine or Vasoxyl, or a pharmaceutically acceptable residue thereof.

43. The composition of claim 40 wherein the cardiovascular agent is Lisinopril, Fosinopril Sodium, Enalaprilat or Captopril, or a pharmaceutically acceptable residue thereof.

44. A method for the treatment or prophylaxis of a cardiovascular disease in a host, comprising administering a therapeutic amount of the compound of any one of the preceding claims 1-38, or its pharmaceutically acceptable salt therein, which comprises an cardiovascular agent.

45. A method for the treatment, prophylaxis and/or diagnosis of a cardiovascular disease in a host, comprising administering an effective amount of the compound of any one of the preceding claims 1-38, or its pharmaceutically acceptable salt therein, which comprises a cardiovascular agent and/or an imaging agent, and optionally detecting the presence of the compound.

46. A method for the diagnosis of a cardiovascular disease in a host, comprising administering to the animal a detectable amount of the compound of any one of the preceding claims 28-38, or its pharmaceutically acceptable salt therein, which comprises an imaging agent and detecting the presence of the compound.

47. A method for the treatment or prophylaxis of a cardiovascular disease in a host, comprising administering a therapeutic amount of a pharmaceutical composition comprising the compound of any one of the preceding claims 1-38, which is contains at least one cardiovascular agent, or its pharmaceutically acceptable salt therein, and a pharmaceutically acceptable carrier.

48. A method for the treatment, prophylaxis and/or diagnosis of a cardiovascular disease in a host, comprising administering an effective amount of a pharmaceutical composition comprising the compound of any one of the preceding claims 1-38, linked to at least one cardiovascular agent and/or imaging agent, or its pharmaceutically acceptable salt therein, and a pharmaceutically acceptable carrier, and optionally detecting the presence of the compound.

49. A method for the diagnosis of a cardiovascular disease in a host, comprising administering a detectable amount of a pharmaceutical composition comprising the compound of any one of the preceding claims 28-38, linked to at least one imaging agent, or its pharmaceutically acceptable salt therein, and a pharmaceutically acceptable carrier, and detecting the presence of the compound.

50. The method of any one of claims 44-49 wherein the cardiovascular disease is arteriosclerotic heart disease, angina pectoris, myocardial infarction, vascular disease, high blood pressure, hypertension, stroke, congestive heart failure, valvular disease, rheumatic heart disease, cardiac arrhythmias, pericarditis, myocarditis, endocarditis, cardiomyopathy, or any combination thereof.
